(19) United States
(12) Patent Application Publication
(10) Pub. No.: US 2013/0116195 A1
(43) Pub. Date: May 9, 2013

(54) Binding ligand linked drug delivery conjugates of tubulysins

(71) Applicants: Christopher Paul Leamon, West Lafayette, IN (US); Iontcho Radoslavov Vlahov, West Lafayette, IN (US)

(72) Inventors: Christopher Paul Leamon, West Lafayette, IN (US); Iontcho Radoslavov Vlahov, West Lafayette, IN (US)

(21) Appl. No.: 13/507,076

(22) Filed: Jun. 1, 2012

Related U.S. Application Data
(63) Continuation of application No. PCT/US2010/058973, filed on Dec. 3, 2010.
(60) Provisional application No. 61/266,751, filed on Dec. 4, 2009.

(51) Int. Cl. A61K 38/08

(52) U.S. Cl. CPC A61K 38/08
USPC 514/19.3

(57) ABSTRACT

Described herein are compounds, pharmaceutical compositions, and methods for treating pathogenic cell populations. Kits including the compounds or pharmaceutical compositions are described. The compounds described herein include conjugates of tubulysins and folates. The conjugates also include a releasable bivalent linker.

### CROSS REFERENCE TO RELATED APPLICATIONS

This application claims the benefit of priority under 35 U.S.C. §119(e) of U.S. provisional patent application Ser. No. 61/266,751 filed Dec. 4, 2009; the disclosure of which are incorporated herein in its entirety by reference.

### TECHNICAL FIELD

The present invention relates to compositions and methods for use in targeted drug delivery. More particularly, the invention is directed to cell-surface receptor binding drug delivery conjugates for use in treating disease states caused by pathogenic cell populations and to methods and pharmaceutical compositions that use and include such conjugates.

### BACKGROUND

The mammalian immune system provides a means for the recognition and elimination of tumor cells, other pathogenic cells, and invading foreign pathogens. While the immune system normally provides a strong line of defense, there are many instances where cancer cells, other pathogenic cells, or infectious agents evade a host immune response and proliferate or persist with concomitant host pathogenicity. Chemotherapeutic agents and radiation therapies have been developed to eliminate, for example, replicating neoplasms. However, many of the currently available chemotherapeutic agents and radiation therapy regimens have adverse side effects because they work not only to destroy pathogenic cells, but they also affect normal host cells, such as cells of the hematopoietic system. The adverse side effects of these anticancer drugs highlight the need for the development of new therapies selective for pathogenic cell populations and with reduced host toxicity.

Researchers have developed therapeutic protocols for destroying pathogenic cells by targeting cytotoxic compounds to such cells. Many of these protocols utilize toxins conjugated to antibodies that bind to antigens unique to or overexpressed by the pathogenic cells in an attempt to minimize delivery of the toxin to normal cells. Using this approach, certain immunotoxins have been developed consisting of antibodies directed to specific antigens on pathogenic cells, the antibodies being linked to toxins such as ricin, Pseudomonas exotoxin, Diphtheria toxin, and tumor necrosis factor. These immunotoxins target pathogenic cells, such as tumor cells, bearing the specific antigens recognized by the antibody (Olsnes, S., Immunol. Today, 10, pp. 291-295, 1989; Melby, E. L., Cancer Res., 53(8), pp. 1755-1760, 1993; Better, M. D., PCT Publication Number WO 91/07418, published May 30, 1991).

Another approach for targeting populations of pathogenic cells, such as cancer cells or foreign pathogens, in a host is to enhance the host immune response against the pathogenic cells to avoid the need for administration of compounds that may also exhibit independent host toxicity. One reported strategy for immunotherapy is to bind antibodies, for example, genetically engineered multimeric antibodies, to the surface of tumor cells to display the constant region of the antibodies on the cell surface and thereby induce tumor cell killing by various immune-system mediated processes (De Vita, V. T., Biologic Therapy of Cancer, 2d ed. Philadelphia, Lippincott, 1995; Soulillou, J. P., U.S. Pat. No. 5,672,486). However, these approaches have been complicated by the difficulties in defining tumor-specific antigens.

Tubulysins are a group of potent inhibitors of tubulin polymerization. Tubulysins are useful in treating diseases and disease states that include pathogenic cell populations, such as cancer. Two particular species of mycobacteria synthesize tubulysins in high titer during fermentation. One species, Archangium gephyra, produces as the main component factors tubulysins A, B, C, G, and I, each of which is characterized by a including the tubutyrosine (Tut, an analog of tyrosine) residue. In contrast, another species, Angiococcus disciformis, produces as the main component factors tubulysins D, E, F, and H, each of which is characterized by-including the tubuphenylalanine (Tup, an analog of phenylalanine) residue. Such bacterial fermentations are convenient sources of tubulysins.

### SUMMARY OF THE INVENTION

In one illustrative embodiment of the invention, a method is provided for treating a patient with cancer, the method comprising the step of administering to the patient a composition comprising a conjugate of a tubulysin of the formula

B-L-D

or a pharmaceutically acceptable salt, isomer, mixture of isomers, crystalline form, non-crystalline form, hydrate, or solvate thereof; wherein

B is a folate;

L is a bivalent linker of the formula

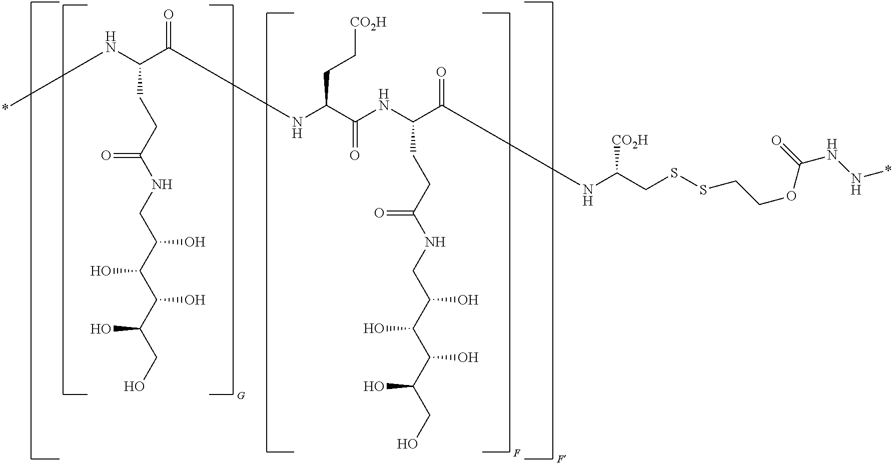

wherein *'s indicate the points of attachment, and F, F′, and G are each independently 1, 2, 3 or 4; and D is a tubulysin.

In another illustrative embodiment of the invention, a method is provided for treating a patient with cancer, the method comprising the step of administering to the patient a composition comprising a conjugate of a tubulysin of the formula

B-L-D

or a pharmaceutically acceptable salt, isomer, mixture of isomers, crystalline form, non-crystalline form, hydrate, or solvate thereof; wherein

B is a folate;

L is a bivalent linker of the formula

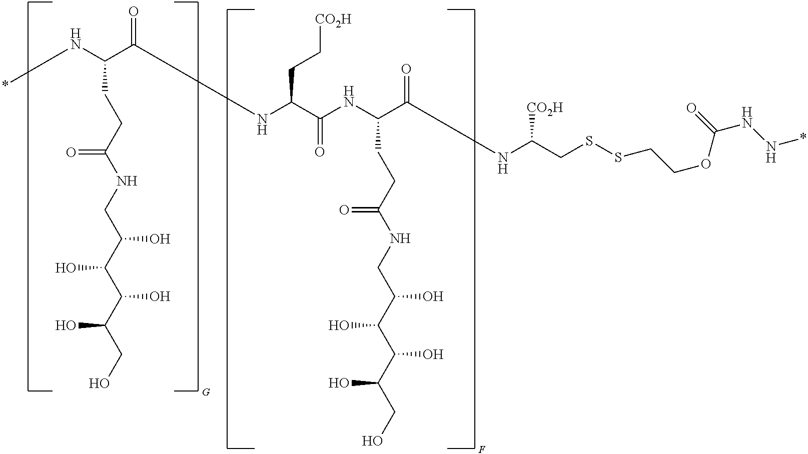

wherein *'s indicate the points of attachment, and F and G are each independently 1, 2, 3 or 4; and D is tubulysin B.

In another embodiment, the method of the preceding embodiments is provided wherein the folate is of the formula

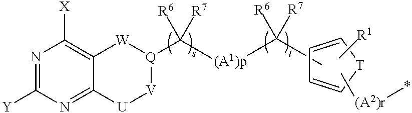

is described wherein * indicates the point of attachment;

X and Y are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡H)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group; and n, p, r, s and t are each independently either 0 or 1.

In another embodiment, the method of any of the preceding embodiments is provided wherein the folate is of the formula

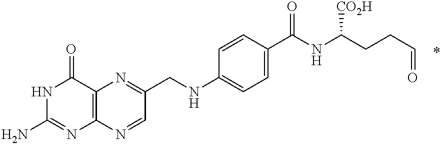

wherein * indicates the point of attachment. In still another embodiment, the method of any of the preceding embodiments wherein F is 2 and G is 1 is described.

In another embodiment, the method of any of the preceding embodiments wherein the conjugate of tubulysin is of the formula

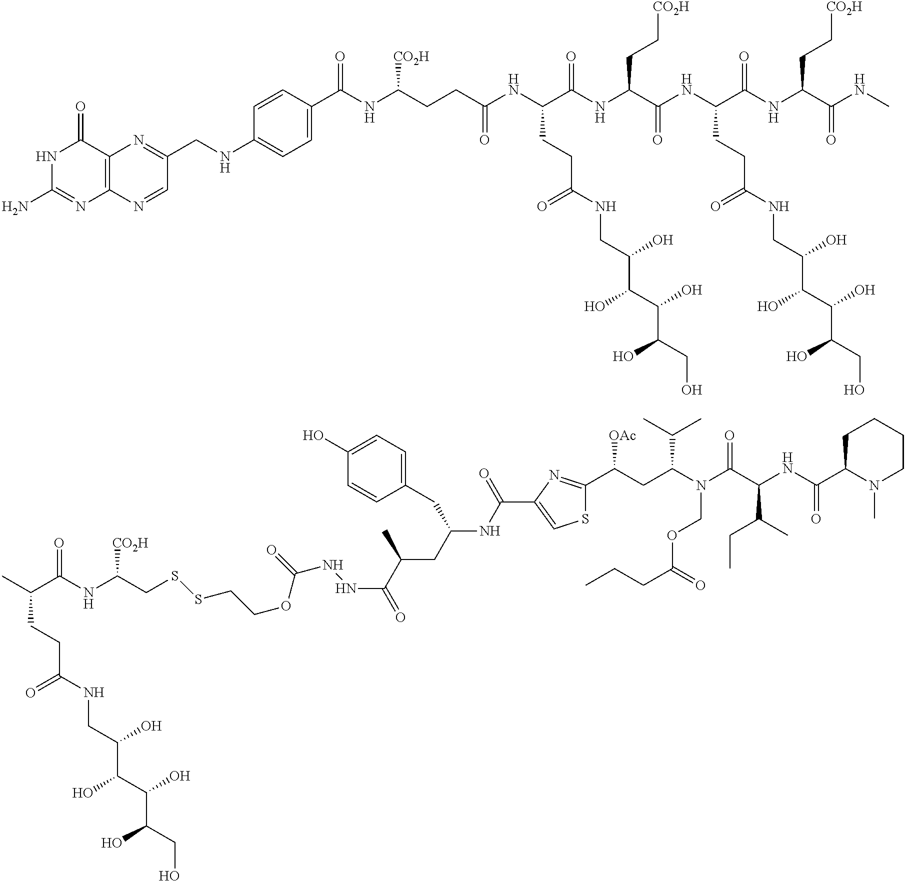

is described.

In another embodiment, the method of any of the preceding embodiments wherein the conjugate of tubulysin is of the formula

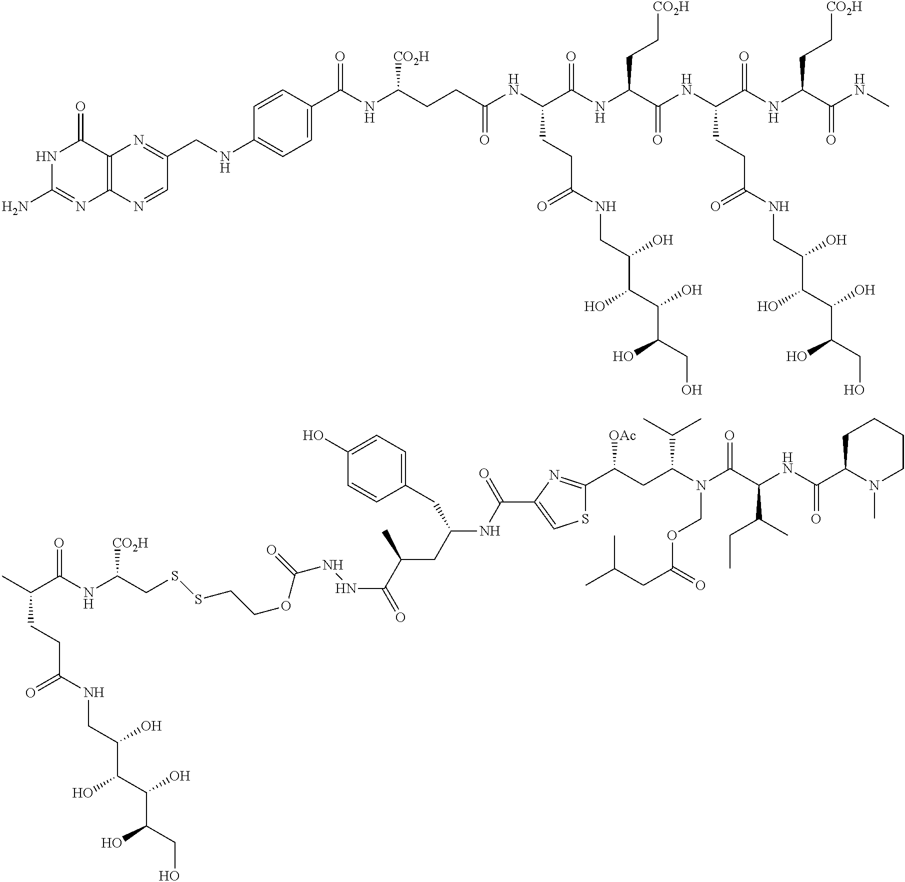

is described.

In another embodiment, the method of any of the preceding embodiments wherein the conjugate of tubulysin is of the formula

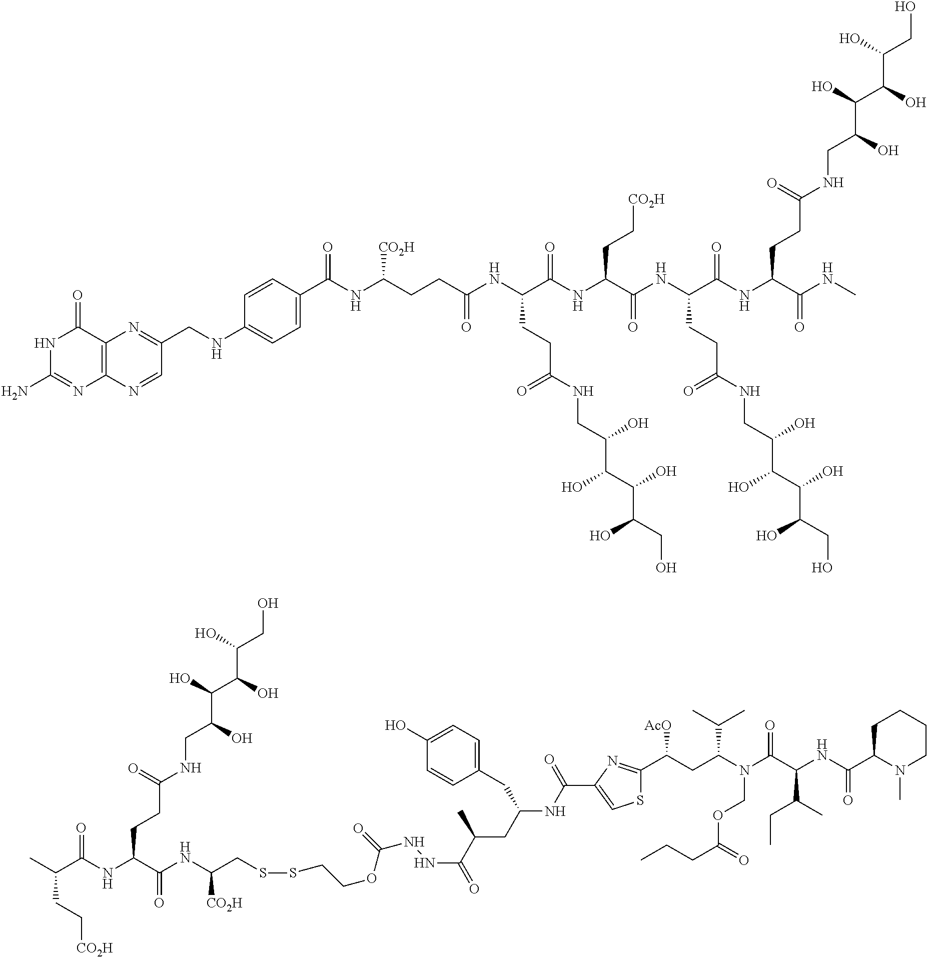 [SMILES: CNC(=O)[C@H](CCC(=O)NC[C@H](O)[C@@H](O)[C@H](O)[C@H](O)CO)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CC[C@H](NC(=O)c1ccc(NCc2cnc3nc(N)[nH]c(=O)c3n2)cc1)C(=O)O ; CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CCC(=O)NC[C@H](O)[C@@H](O)[C@H](O)[C@H](O)CO)NC(=O)[C@@H](C)CCC(=O)O)C(=O)O)cs1)C(C)C]

is described.

In another embodiment, the method of any of the preceding embodiments wherein the conjugate of tubulysin is of the formula

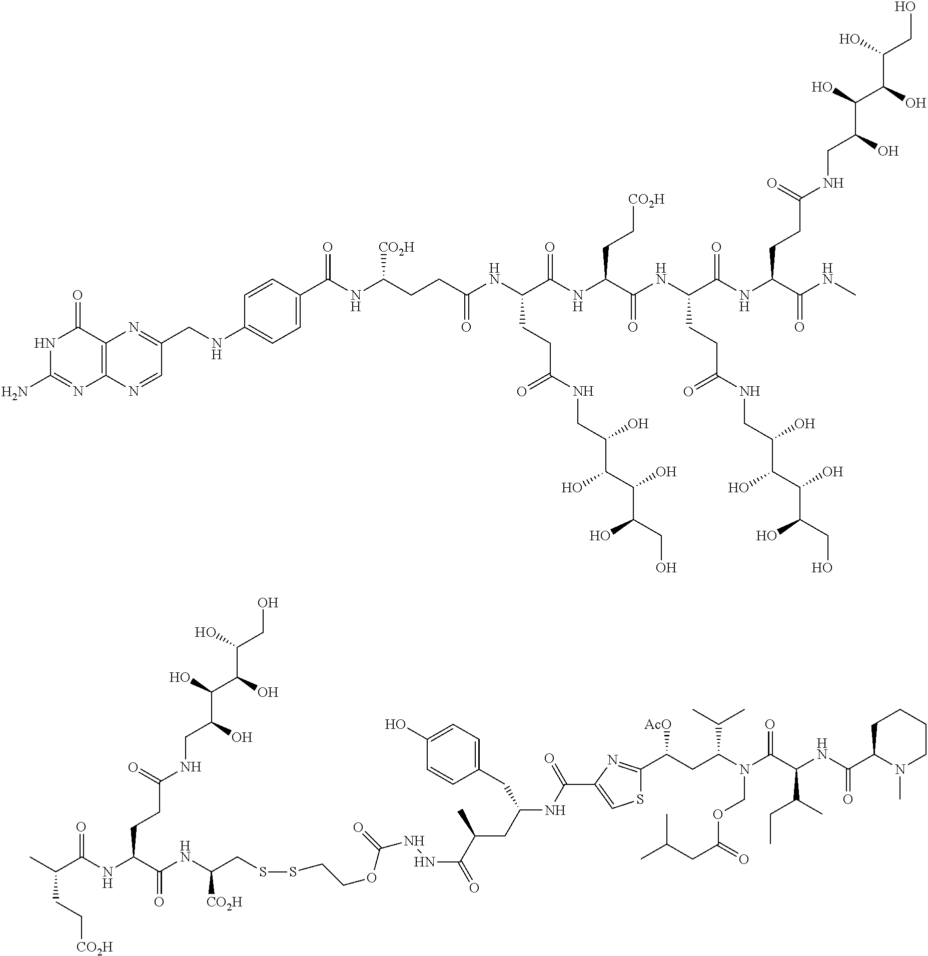 [SMILES: CNC(=O)[C@H](CCC(=O)NC[C@H](O)[C@@H](O)[C@H](O)[C@H](O)CO)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CC[C@H](NC(=O)c1ccc(NCc2cnc3nc(N)[nH]c(=O)c3n2)cc1)C(=O)O ; CCC(C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)NNC(=O)OCCSSC[C@H](NC(=O)[C@H](CCC(=O)NC[C@H](O)[C@@H](O)[C@H](O)[C@H](O)CO)NC(=O)[C@@H](C)CCC(=O)O)C(=O)O)cs1)C(C)C]

is described.

In another embodiment, the method of any of the preceding embodiments wherein the composition further comprises one or more carriers, diluents, or excipients, or a combination thereof is described. In another embodiment, the method of any of the preceding embodiments wherein the purity of the conjugate of tubulysin is at least 98% is described.

In another embodiment, the method of any of the preceding embodiments wherein the composition is in a dosage form adapted for parenteral administration is described. In another embodiment, the method of any of the preceding embodiments wherein the dose of the conjugate of tubulysin is in the range of 1 to 5 μg/kg is described. In another embodiment, the method of any of the preceding embodiments wherein the dose of the conjugate of tubulysin is in the range of 1 to 3 μg/kg is described.

In another embodiment, a kit comprising a sterile vial, the composition of any one of the preceding claims, and instructions for use describing use of the composition for treating a patient with cancer is described.

In another embodiment, the kit of the preceding embodiment wherein the composition is in the form of a reconstitutable lyophlizate is described.

In another embodiment, the method of any of the preceding kit embodiments wherein the dose of the conjugate of tubulysin is in the range of 1 to 5 μg/kg is described.

In another embodiment, the method of any of the preceding kit embodiments wherein the dose of the conjugate of tubulysin is in the range of 1 to 3 μg/kg is described.

In another embodiment, the method of any of the preceding kit embodiments wherein the purity of the conjugate of tubulysin is at least 98% is described.

### BRIEF DESCRIPTION OF THE DRAWINGS

FIG. 1 shows the relative affinity assay results in 10% serum/FDRPMI for EC0531: () folic acid, relative affinity=1; (▪) EC0531, relative affinity=0.49.

FIG. 2A shows the activity of EC0531 against KB cells following a 2 hour pulse and a 72 hour chase. The IC₅₀ for EC0531 was about 2.4 nM. In addition, the cytotoxic activity of EC0531 was blocked in the presence of an excess of a folate-fluorescein conjugate.

FIG. 2B shows the activity of EC0530 against KB cells following a 2 hour pulse and a 72 hour chase. The IC₅₀ for EC0530 was about 2.2 nM. In addition, the cytotoxic activity of EC0530 was blocked in the presence of an excess of a folate-fluorescein conjugate.

FIG. 2C shows the activity of EC0533 against KB cells following a 2 hour pulse and a 72 hour chase. The IC₅₀ for EC0533 was about 5.6 nM. In addition, the cytotoxic activity of EC0533 was blocked in the presence of an excess of a folate-fluorescein conjugate.

FIG. 2D shows the activity of EC0543 against KB cells following a 2 hour pulse and a 72 hour chase. The IC₅₀ for EC0543 was about 1 nM. In addition, the cytotoxic activity of EC0543 was blocked in the presence of an excess of a folate-fluorescein conjugate.

FIG. 3A shows tumor volume (mm³) in treated animals, as compared to controls for various doses of unconjugated tubulysin B: () 1 μmol/kg (2 doses); (▴) 0.75 μmol/kg; (♦) 0.5 μmol/kg; (▪) KB Controls, untreated.

FIG. 3B shows the percent weight change in treated animals, as compared to controls for various doses of unconjugated tubulysin B: () 1 μmol/kg (2 doses); (▴) 0.75 μmol/kg; (♦) 0.5 μmol/kg; (▪) KB Controls untreated.

FIG. 4A shows tumor volume (mm³) in treated animals, as compared to controls for various doses of EC0531: () 3 μmol/kg; (♦) 2 μmol/kg; (▾) 1 μmol/kg; (▪) KB Controls, untreated. The vertical dotted line indicates the day of final dosing.

FIG. 4B shows the percent weight change in treated animals, as compared to controls for various doses of EC0531: () 3 μmol/kg; (♦) 2 μmol/kg; (▾) 1 μmol/kg; (▪) KB Controls, untreated. The vertical dotted line indicates the day of final dosing.

FIG. 5A shows the tumor volume (mm³) in animals treated with EC0531 or its non-sugar counterpart, EC0305. The number of partial responses (PR) out the total number of treated animals is shown: (♦) EC0531 (4/5 PR); () EC0305 (0/5 PR); (▪) KB Control. Panel B: () EC0305; (♦) EC0531; (▪) KB Control, untreated. The vertical dotted line indicates the day of final dosing.

FIG. 5B shows % weight change for animals treated with EC0531 or its non-sugar counterpart, EC0305. () EC0305; (♦) EC0531; (▪) KB Control, untreated. The vertical dotted line indicates the day of final dosing. The vertical dotted line indicates the day of final dosing.

FIG. 6 shows the percent weight change in treated animals, as compared to controls, after treatment with multiple doses of EC0305, EC0510, and EC0531 at 3 μmol/kg (TIW) in female mice: (♦) EC0531 (safe and tolerable); (▾) EC0510 (exceeded MTD); (▴) EC0305 (exceeded MTD); (▪) Controls. The vertical dotted lines indicate the days of final dosing.

FIG. 7 shows the total tumor volume for subcutaneous M109 tumors in Balb/c mice. The ratio of complete responses (CR) to the number of treated animals are shown. (a) untreated controls; (b) treated with EC0436, 2 umol/kg, TIW, 2 weeks (5/5); (c) treated with EC0305, 2 umol/kg, TIW, 2 weeks (4/5).

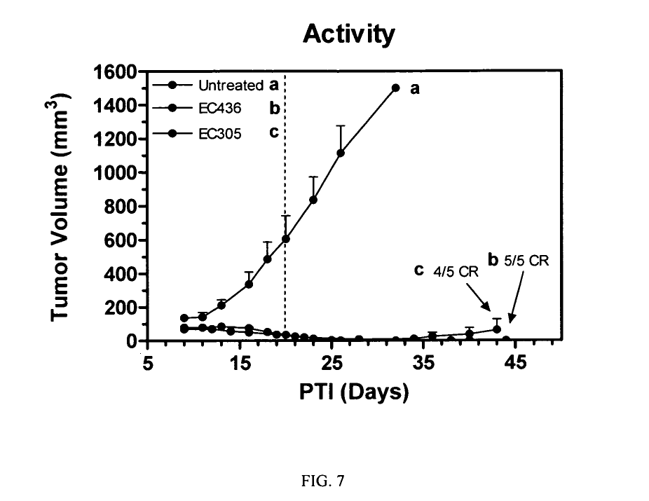

FIG. 8 shows the percentage weight change for mice treated as described for FIG. 6AA, (a) untreated controls; (b) treated with EC0436, 2 umol/kg, TIW, 2 weeks; (c) treated with EC0305, 2 umol/kg, TIW, 2 weeks.

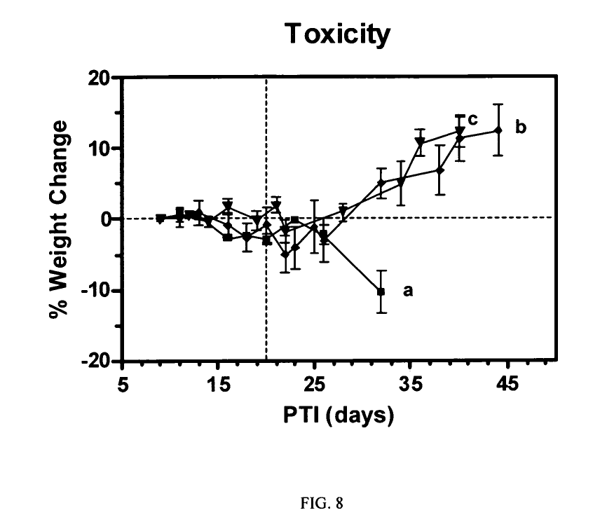

FIG. 9 Shows the effect of several EC0531 and EC0543 doses on s. c. KB tumor growth in nu/nu mice. Randomized nu/nu mice with KB tumors (112-198 mm³ range) were treated (q2d, 6 doses) with various doses (1, 2 or 3 μmol/kg,) of either EC0531 or EC0543. a) control, no treatment {0, 0, 0}; b) EC0531 at 1 μmol/kg {0, 0, 5}; c) EC0531 at 2 μmol/kg {0, 0, 5}; d) EC0531 at 3 μmol/kg {0, 0, 4}; e) EC0543 at 1 μmol/kg {0, 0, 5}; f) EC0543 at 2 μmol/kg {0, 0, 5}; and g) EC0543 at 3 μmol/kg {0, 0, 4}; Due to the observed weight loss (˜15 to 18%) in this group, EC0543 at 3 μmol/kg was only dosed 3 times. Both, EC0531 and EC0543 resulted in tumor free mice at all the three doses tested.

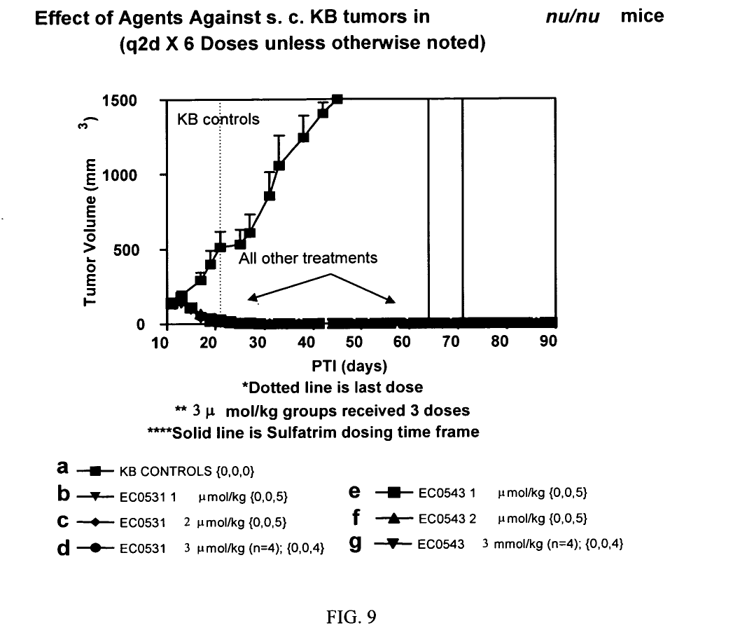

FIG. 10 Effect of various spacers on the antitumor activity of folate-tubulysin conjugates on KB tumors in nu/nu mice. Randomized nu/nu mice with KB tumors (105-195 mm³ range) were treated with non-curable doses (0.5 μmol/kg, q2d, 6 doses) of a folate-tubulysin conjugate, 5 mice per treatment. Individual tumor scores are shown for each treatment group {partial response, complete response, cures}. a) control group, no treatment {0, 0, 0}; b) a folate-tubulysin B conjugate with 4 sugar units (EC0530) {3, 1, 0}; c) a folate-tubulysin B conjugate with 3 sugar units (EC0531) {4, 0, 0}; d) a folate-tubulysin A conjugate 4 sugar units (EC0533) {1, 1, 3}; e) folate-tubulysin A conjugate with 0 sugar units (EC0510) {1, 3, 1}; f) a folate-tubulysin A conjugate with 3 sugar units (EC0543) {0, 2, 3}; and g) a folate-tubulysin B conjugate with 0 sugar units (EC0305) {0, 0, 0}. The tubulysin A conjugates were generally more potent than the tubulysin B conjugates at equimolar doses. However, unexpected random toxicities were observed in the tubulysin A groups and not in the tubulysin B groups.

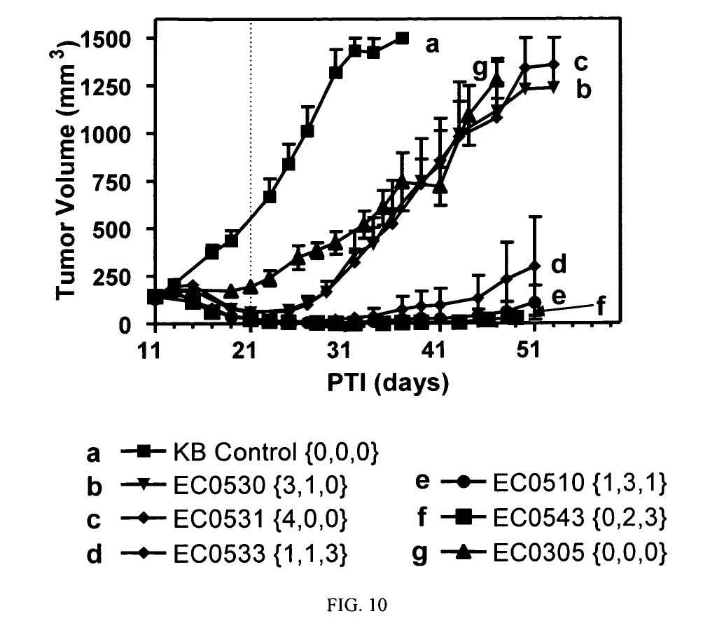

### DETAILED DESCRIPTION

Drug delivery conjugates are described herein consisting of a binding ligand (B), a bivalent linker (L), and a tubulysin (D), including analogs and derivatives thereof. The binding ligand (B) is covalently attached to the bivalent linker (L), and the tubulysin, or analog or derivative thereof, is also covalently attached to the bivalent linker (L). The bivalent linker (L) comprises one or more spacer linkers and/or releasable linkers, and combinations thereof, in any order. In one variation, releasable linkers, and optional spacer linkers are covalently bonded to each other to form the linker. In another variation, a releasable linker is directly attached to the tubulysin, or analog or derivative thereof. In another variation, a releasable linker is directly attached to the binding ligand. In another variation, either or both the binding ligand and the tubulysin, or analog or derivative thereof, is attached to a releasable linker through one or more spacer linkers. In another variation, each of the binding ligand and the tubulysin, or analog or derivative thereof, is attached to a releasable linker, each of which may be directly attached to each other, or covalently attached through one or more spacer linkers. From the foregoing, it should be appreciated that the arrangement of the binding ligand, and the tubulysin, or analog or derivative thereof, and the various releasable and optional spacer linkers may be varied widely. In one aspect, the binding ligand, and the tubulysin, or analog or derivative thereof, and the various releasable and optional spacer linkers are attached to each other through heteroatoms, such as nitrogen, oxygen, sulfur, phosphorus, silicon, and the like. In variations, the heteroatoms, excluding oxygen, may be in various states of oxidation, such as N(OH), S(O), S(O)₂, P(O), P(O)₂, P(O)₃, and the like. In another variation, the heteroatoms may be grouped to form divalent radicals, such as for example hydroxylamines, hydrazines, hydrazones, sulfonates, phosphinates, phosphonates, and the like.

In one aspect, the receptor binding ligand (B) is a vitamin, or analog or derivative thereof, or another vitamin receptor binding compound.

As used herein, tubulysins refer generally to tetrapeptide compounds of the formula

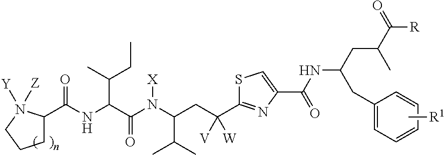

and pharmaceutical salts thereof, where

n is 1-3;

V is hydrogen, OR², or halo, and W is hydrogen, OR², or alkyl, where R² is independently selected in each instance from hydrogen, alkyl, and C(O)R³, where R³ is alkyl, cycloalkyl, alkenyl, aryl, or arylalkyl, each of which is optionally substituted; providing that R² is not H when both V and W are OR²; or V and W are taken together with the attached carbon to form a carbonyl;

X is hydrogen, or C₁₋₄ alkyl or alkenyl, each of which is optionally substituted, or CH₂QR⁹; where Q is —N—, —O—, or —S—; R⁹ is hydrogen or C₁₋₄ alkyl, alkenyl, aryl, or C(O)R¹⁰; and R¹⁰=C₁₋₆ alkyl, alkenyl, aryl, or heteroaryl, each of which is optionally substituted;

Z is alkyl and Y is O; or Z is alkyl or C(O)R⁴, and Y is absent, where R⁴ is alkyl, CF₃, or aryl;

R¹ is hydrogen, or R¹ represents 1 to 3 substituents selected from halo, nitro, carboxylate or a derivative thereof, cyano, hydroxyl, alkyl, haloalkyl, alkoxy, haloalkoxy, phenol protecting groups, prodrug moieties, and OR⁶, where R⁶ is optionally substituted aryl, C(O)R⁷, P(O)(OR⁸)₂, or SO₃R⁸, where R⁷ and R⁸ are independently selected in each instance from hydrogen, or the group consisting of alkyl, alkenyl, cycloalkyl, heterocyclyl, aryl, and arylalkyl, each of which is optionally substituted, or R⁸ is a metal cation; and

R is OH or a leaving group, or R forms a carboxylic acid derivative.

Conjugates of each of the foregoing tubulysins are described herein. In one variation, Z is methyl. In another variation, R¹ is H. In another variation, R¹ is OR⁶ at C(4), where R⁶ is hydrogen, alkyl, or COR⁷. In another variation, V is hydrogen, and W is OC(O)R³.

In another embodiment, conjugates of tubulysins of the following general formula are described

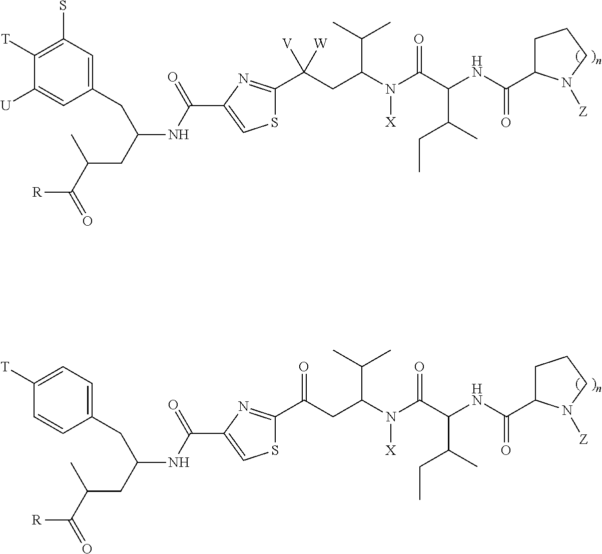

and pharmaceutical salts thereof, where

n is 1-3;

V is hydrogen, OR², or halo, and W is hydrogen, OR², or alkyl, where R² is independently selected in each instance from hydrogen, alkyl, or C(O)R³, where R³ is alkyl, alkenyl or aryl, providing that R² is not H when both V and W are OR²; or V and W are taken together with the attached carbon to form a carbonyl;

X=H, C₁₋₄ alkyl, alkenyl, each of which is optionally substituted, or CH₂QR⁹;

where Q is —N—, —O—, or —S—; R⁹=H, C₁₋₄ alkyl, alkenyl, aryl, or C(O)R¹⁰, and R¹⁰=C₁₋₆ alkyl, alkenyl, aryl, or heteroaryl, each of which is optionally substituted;

Z is alkyl or C(O)R⁴, where R⁴ is alkyl, CF₃, or aryl;

T is hydrogen or OR⁶, where R⁶ is hydrogen, alkyl, aryl, COR⁷, P(O)(OR⁸)₂, or SO₃R⁸, where R⁷ and R⁸ are independently selected in each instance from hydrogen or the group consisting of alkyl, alkenyl, cycloalkyl, heterocyclyl, aryl, and arylalkyl, each of which is optionally substituted, or R⁸ is a metal cation, or R⁶ is a phenol protecting group, or a prodrug moiety;

S and U are each independently selected from the group consisting of H, halo, nitro, cyano, alkyl, haloalkyl, alkoxy, and haloalkoxy; and

R is OH or a leaving group, or R forms a carboxylic acid derivative.

In one variation, Z is methyl or C(O)R⁴.

Natural tubulysins are generally linear tetrapeptides consisting of N-methyl pipecolic acid (Mep), isoleucine (Ile), an unnatural aminoacid called tubuvalin (Tuv), and either an unnatural aminoacid called tubutyrosine (Tut, an analog of tyrosine) or an unnatural aminoacid called tubuphenylalanine (Tup, an analog of phenylalanine). In another embodiment, naturally occurring tubulysins, and analogs and derivatives thereof, of the following general formula are described

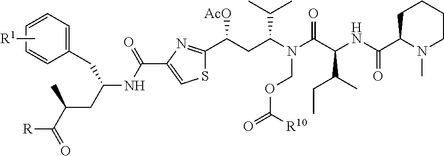

and pharmaceutical salts thereof, where R, R¹, and R¹⁰ are as described in the various embodiments herein. Conjugates of each of the foregoing tubulysins are described herein.

In another embodiment, conjugates of naturally occurring tubulysins of the following general formula are described

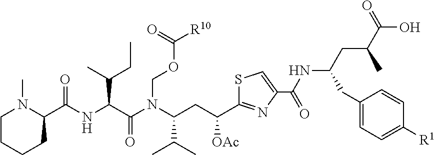

| Factor | R¹⁰ | R¹ |
|---|---|---|
| A | (CH₃)₂CHCH₂ | OH |
| B | CH₃(CH₂)₂ | OH |
| C | CH₃CH₂ | OH |
| D | (CH₃)₂CHCH₂ | H |
| E | CH₃(CH₂)₂ | H |
| F | CH₂CH₃ | H |
| G | (CH₃)₂C═CH | OH |
| H | CH₃ | H |
| I | CH₃ | OH |

and pharmaceutical salts thereof.

In another embodiment, conjugates of tubulysins of the following formula are described:

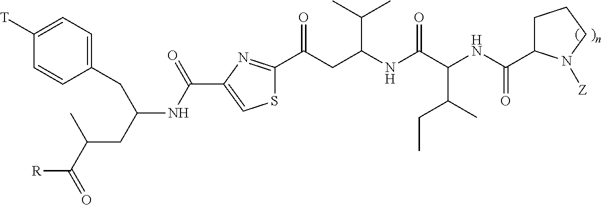

and pharmaceutical salts thereof, where n is 1-3; T is hydrogen or OR⁶, where R⁶ is hydrogen, alkyl, aryl, COR⁷, P(O)(OR⁸)₂, or SO₃R⁸, where R⁷ and R⁸ are independently selected in each instance from hydrogen, or the group consisting of alkyl, alkenyl, cycloalkyl, heterocyclyl, aryl, and arylalkyl, each of which is optionally substituted, or R⁸ is a metal cation, or R⁶ is a phenol protecting group, or a prodrug moiety; Z is alkyl or C(O)R⁴, where R⁴ is alkyl, CF₃, or aryl; and R is OH or a leaving group, or R forms a carboxylic acid derivative. Illustrative examples of such compounds, and their preparation are described in J. Med. Chem. 10.1021/jm701321p (2008), the disclosure of which is incorporated herein by reference.

In another embodiment, conjugates of tubulysins of the following formula are described:

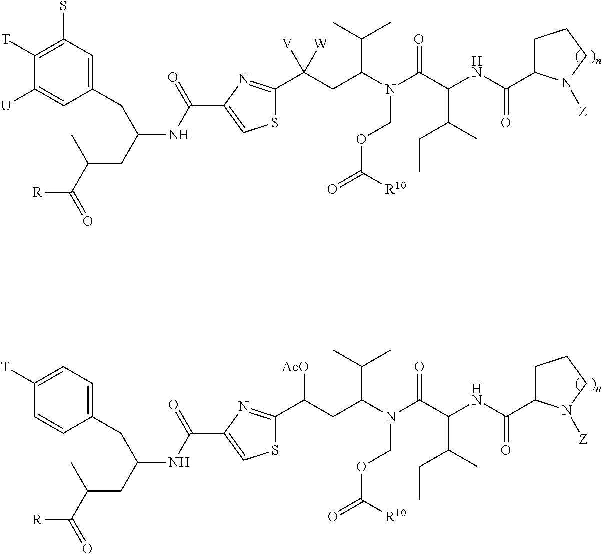

and pharmaceutical salts thereof, where n, S, T, U, V, W, Z, R, and R¹⁰ are as described in the various embodiments herein.

In another embodiment, conjugates of tubulysins of the following formula are described:

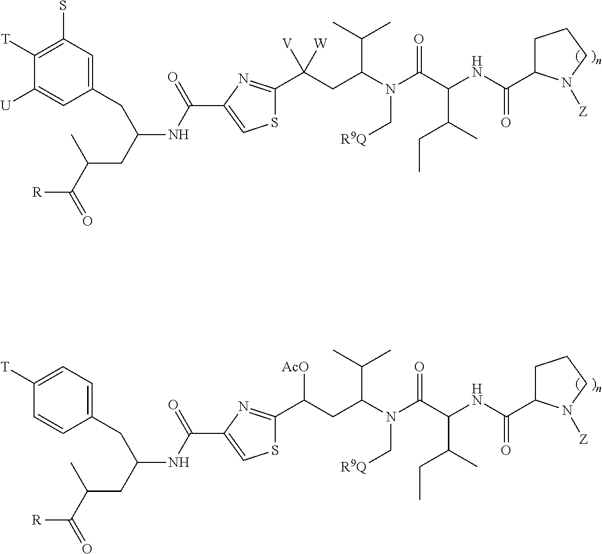

and pharmaceutical salts thereof, where n, S, T, U, V, W, Z, QR⁹, and R are as described in the various embodiments herein. In one variation, Q is —N—, —O—, or —S—; and R⁹ is hydrogen, alkyl, alkenyl, cycloalkyl, aryl, or arylalkyl, each of which is optionally substituted. In another variation, QR⁹ are taken together to form C(O)R¹⁰, S(O)₂R¹⁰, P(O)(OR^{10a})₂, where R¹⁰ and OR^{10a} are independently selected in each instance from the group consisting of H, alkyl, alkenyl, cycloalkyl, aryl, and arylalkyl, each of which is optionally substituted, or R^{10a} is a metal cation.

In another embodiment, conjugates of tubulysins of the following formula are described:

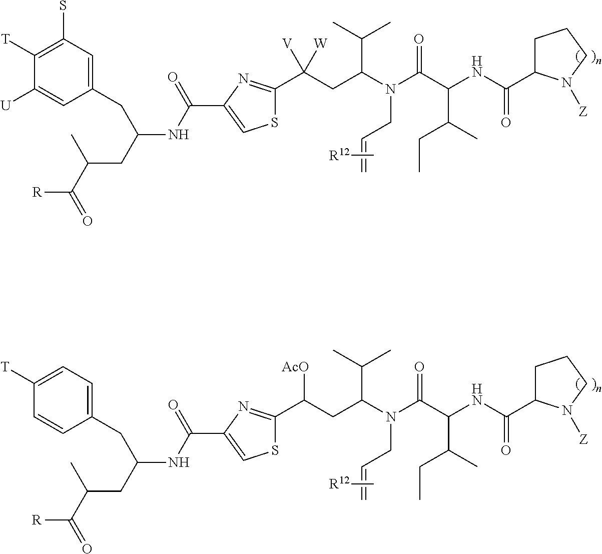

and pharmaceutical salts thereof, where R¹² represents 1 or more substituents selected from alkyl, alkenyl, cycloalkyl, aryl, and arylalkyl, each of which is optionally substituted; and where n, S, T, U, V, W, Z, and R are as described in the various embodiments herein. It is to be understood that other olefins may form by isomerization, depending on the conditions of the reaction and the identity of R¹. For example, when R¹ is alkyl, it is appreciated that under the reaction conditions, the double bond can migrate to other carbon atoms along the alkenyl chain, including to form the terminal or co-olefin.

In another embodiment, conjugates of tubulysins of the following formula are described:

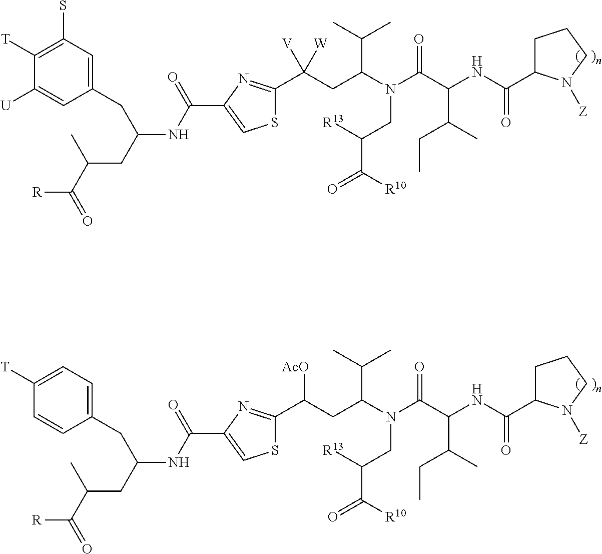

and pharmaceutical salts thereof, where R¹³ is C(O)R¹⁰, C(O)OR¹⁰ or CN; and where n, S, T, U, V, W, Z, R, and R¹⁰ are as described in the various embodiments herein, where R¹⁰ is independently selected in each instance.

In another embodiment, conjugates of tubulysins of the following formula are described:

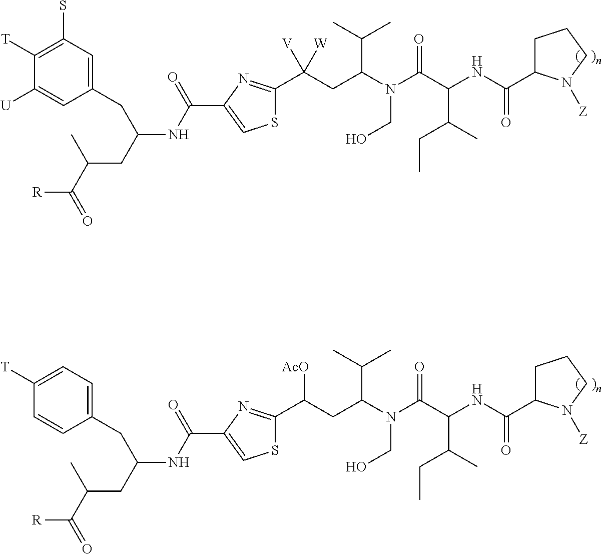

and pharmaceutical salts thereof, where n, S, T, U, V, W, Z, and R are as described in the various embodiments herein.

In another embodiment, conjugates of tubulysins of the following formula are described:

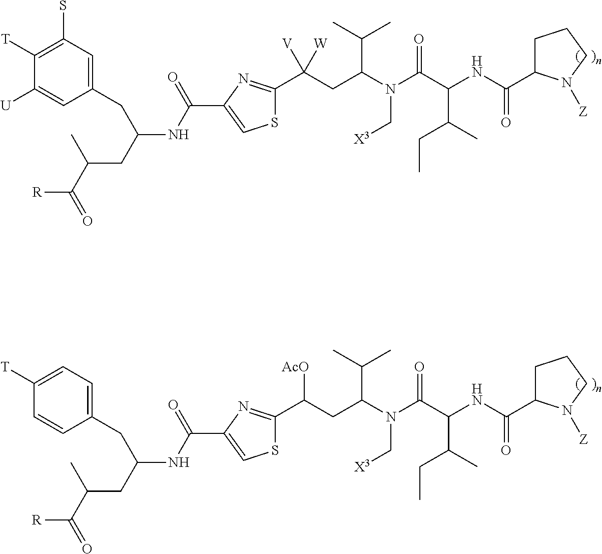

and pharmaceutical salts thereof, where X³ is halogen, OS(O)₂R¹⁰, OP(O)(OR^{10a})R¹⁰, or OP(O)(OR^{10a})₂; where R¹⁰ and R^{10a} are independently selected in each instance from hydrogen or the group consisting of alkyl, alkenyl, cycloalkyl, aryl, and arylalkyl, each of which is optionally substituted, or R^{10a} is a metal cation; and where n, S, T, U, V, W, Z, and R are as described in the various embodiments herein.

Additional tubulysins useful in preparing the conjugates described herein are described in US patent application publication Nos. 2006/0128754 and 2005/0239713, the disclosures of which are incorporated herein by reference. Additional tubulysins useful in preparing the conjugates described herein are described in U.S. provisional application Serial Nos. 60/982,595 and 61/036,176 and now combined in U.S. patent application publication No. 2010/0240701, the disclosures of which are incorporated herein by reference. Tubulysins may also be prepared as described in Peltier et al., “The Total Synthesis of Tubulysin D,” J. Am. Chem. Soc. 128:16018-19 (2006), the disclosure of which is incorporated herein by reference.

In each of the foregoing embodiments, it is understood that in one variation, the compounds of the various formulae have the following absolute configuration:

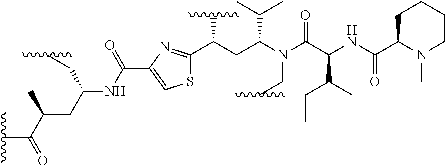 [SMILES: *C[C@@H](C[C@H](C)C(*)=O)NC(=O)c1csc([C@H](*)C[C@H](C(C)C)N(C*)C(=O)[C@@H](NC(=O)[C@H]2CCCCN2C)C(C)CC)n1]

at the indicated asymmetric backbone carbon atoms.

It is to be understood that the conjugate of the tubulysin or analog or derivative thereof may be formed at any position. Illustratively, conjugates of tubulysins are described where the bivalent linker (L) is attached to any of the following positions:

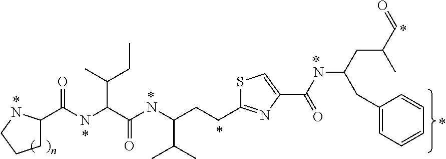

where the (*) symbol indicates optional attachment locations.

In another embodiment, the conjugates are formed from carboxylic acid derivatives of the tubulysin, or an analog or derivative thereof. Illustrative carboxylic acid conjugate derivatives of the tubulysin are represented by the following general formula

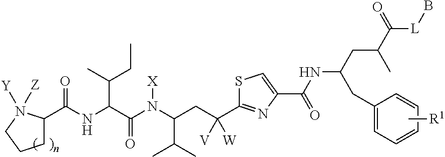

and pharmaceutical salts thereof, where

B is a binding ligand;

L is a linker; where L includes a heteroatom linker covalently attached to the tubulysin, such as an oxygen, nitrogen, or sulfur heteroatom;

n is 1-3;

V is hydrogen, OR², or halo, and W is hydrogen, OR², or alkyl, where R² is independently selected in each instance from hydrogen, alkyl, or C(O)R³, where R³ is alkyl, alkenyl or aryl, providing that R² is not H when both V and W are OR²; or V and W are taken together with the attached carbon to form a carbonyl;

X=H, C₁₋₄ alkyl, alkenyl, each of which is optionally substituted, or CH₂QR⁹; where Q is —N—, —O—, or —S—; R⁹=H, C₁₋₄ alkyl, alkenyl, aryl, or C(O)R¹⁰; and R¹⁰=C₁₋₆ alkyl, alkenyl, aryl, or heteroaryl, each of which is optionally substituted;

Z is alkyl and Y is O; or Z is alkyl or C(O)R⁴, and Y is absent, where R⁴ is alkyl, CF₃, or aryl;

R¹ is hydrogen, or R¹ represents 1 to 3 substituents selected from halo, nitro, carboxylate or a derivative thereof, cyano, hydroxyl, alkyl, haloalkyl, alkoxy, haloalkoxy, phenol protecting groups, prodrug moieties, and OR⁶, where R⁶ is optionally substituted aryl, C(O)R⁷, P(O)(OR⁸)₂, or SO₃R⁸, where R⁷ and R⁸ are independently selected in each instance from hydrogen, or the group consisting of alkyl, alkenyl, cycloalkyl, heterocyclyl, aryl, and arylalkyl, each of which is optionally substituted, or R⁸ is a metal cation; and

R is OH or a leaving group, or R forms a carboxylic acid derivative.

In another embodiment, illustrative carboxylic acid conjugate derivatives of tubulysin of the following general formula are described

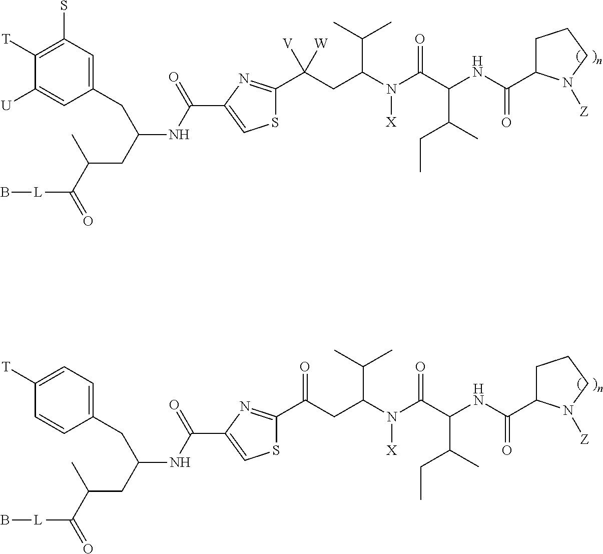

and pharmaceutical salts thereof, where

B is a binding ligand;

L is a linker; where L includes a heteroatom linker covalently attached to the tubulysin, such as an oxygen, nitrogen, or sulfur heteroatom;

n is 1-3;

V is hydrogen, OR², or halo, and W is hydrogen, OR², or alkyl, where R² is independently selected in each instance from hydrogen, alkyl, or C(O)R³, where R³ is alkyl, alkenyl or aryl, providing that R² is not H when both V and W are OR²; or V and W are taken together with the attached carbon to form a carbonyl;

X=H, C₁₋₄ alkyl, alkenyl, each of which is optionally substituted, or CH₂QR⁹; where Q is —N—, —O—, or —S—; R⁹=H, C₁₋₄ alkyl, alkenyl, aryl, or C(O)R¹⁰; and R¹⁰=C₁₋₆ alkyl, alkenyl, aryl, or heteroaryl, each of which is optionally substituted;

Z is alkyl or C(O)R⁴, where R⁴ is alkyl, CF₃, or aryl;

T is hydrogen or OR⁶, where R⁶ is hydrogen, alkyl, aryl, COR⁷, P(O)(OR⁸)₂, or SO₃R⁸, where R⁷ and R⁸ are independently selected in each instance from hydrogen, or the group consisting of alkyl, alkenyl, cycloalkyl, heterocyclyl, aryl, and arylalkyl, each of which is optionally substituted, or R⁸ is a metal cation, or R⁶ is a phenol protecting group, or a prodrug moiety;

S and U are each independently selected from the group consisting of H, halo, nitro, cyano, alkyl, haloalkyl, alkoxy, and haloalkoxy; and

R is OH or a leaving group, or R forms a carboxylic acid derivative.

In another embodiment, illustrative carboxylic acid conjugate derivatives of the following general formulae are described

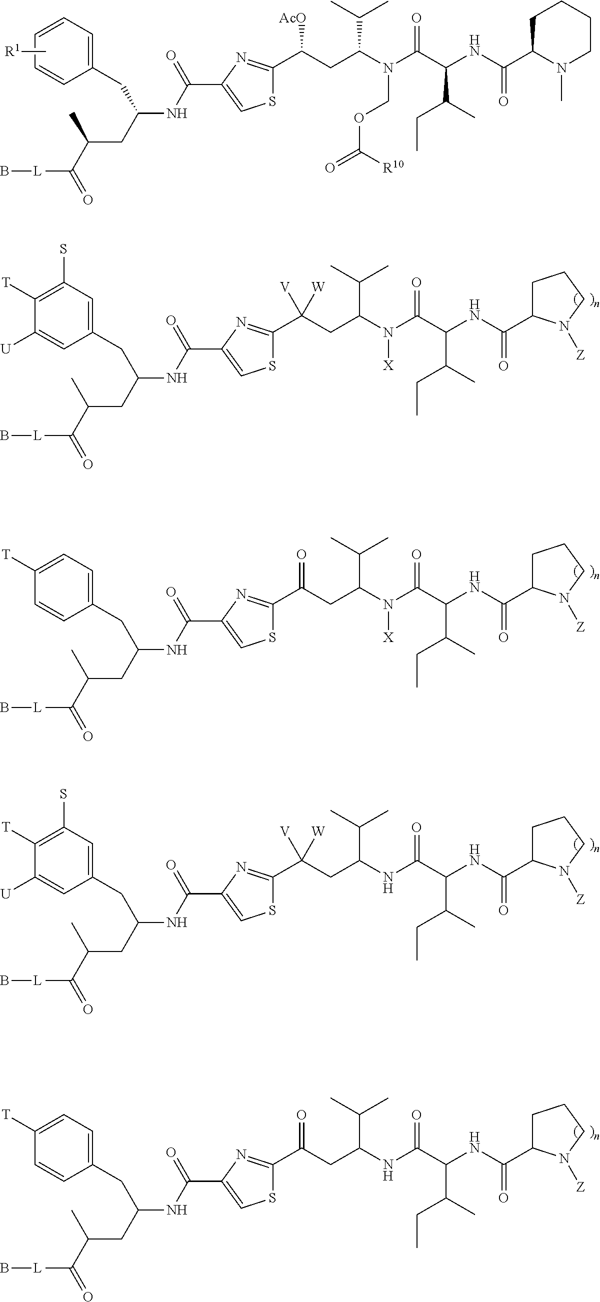

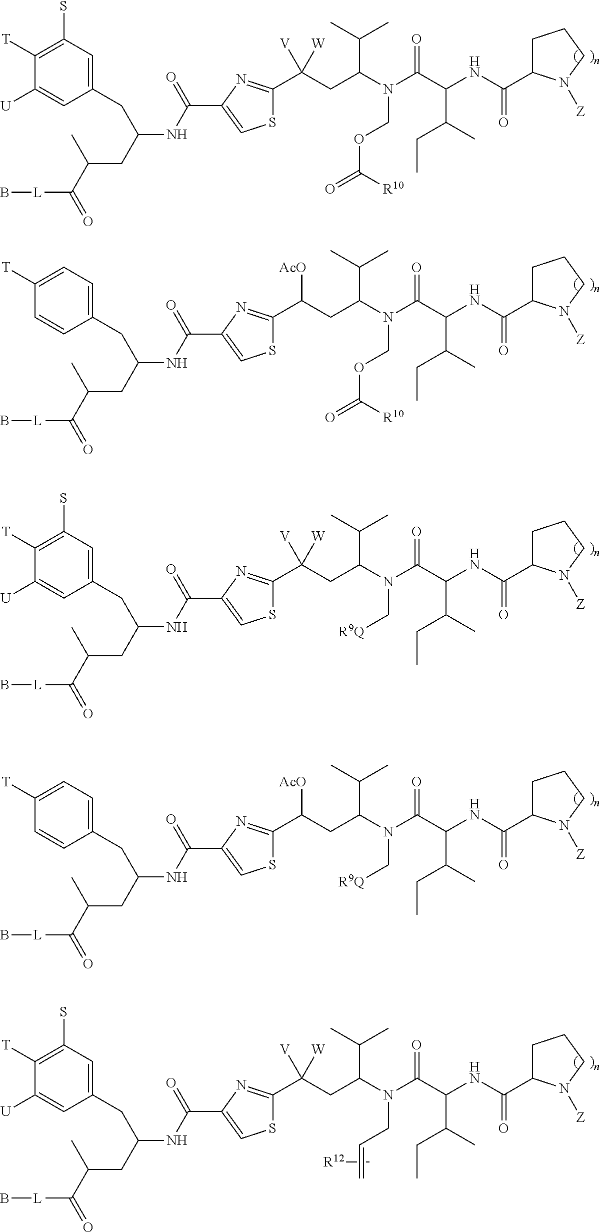

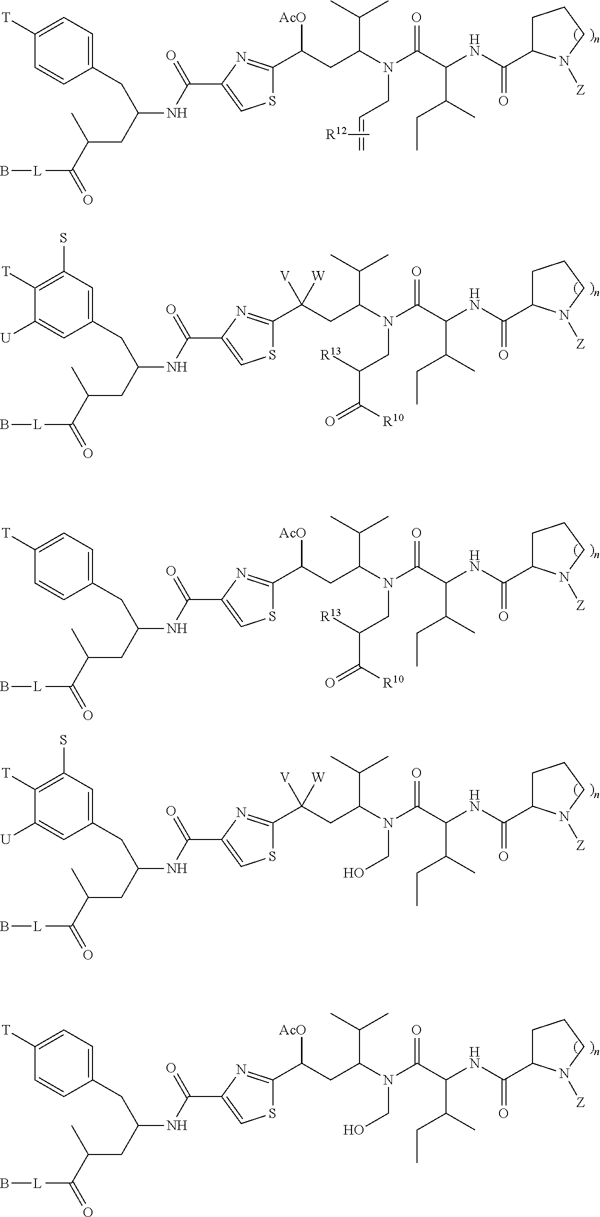

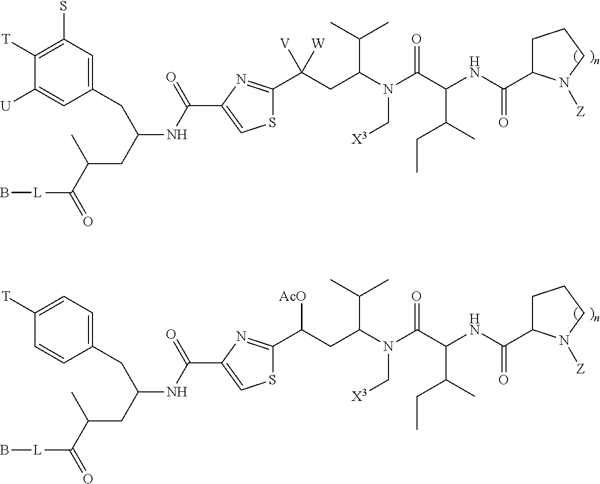

and pharmaceutical salts thereof, where B, L, n, S, T, U, V, W, X, Z, Q, R¹, R⁹, R¹⁰, R¹², R¹³, and X³ are as described herein in the various embodiments and aspects.

In another embodiment, illustrative carboxylic acid conjugate derivatives of naturally occurring tubulysins such as tubulysin A, tubulysin B, and tubulysin I, are described, and pharmaceutical salts thereof.

In another embodiment, illustrative carboxylic acid conjugate derivatives of the following tubulysin analogs and derivatives are described

Additional tubulysins that are useable in the conjugates described herein include the following:

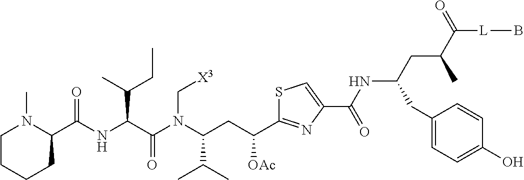

| Conjugate | X³ |
|---|---|
| B-L-EC0313 | —O—CH₃ |
| B-L-EC0346 | —O—(CH₂)₂—OH |
| B-L-EC0356 | —O—(CH₂)₂CH(CH₃)₂ |
| B-L-EC0374 | —S—(CH₂)₂—SH |
| B-L-EC0386 | —OH |
| B-L-EC0550 | —(CH₂)₂—CH═CH₂ |
| B-L-EC0560 | —S—(CH₂)₂—OH |
| B-L-EC0575 | —O—C(O)—(CH═CH)—CH₂—Cl |
| B-L-EC0585 | —NH—C(O)—CH₂CH(CH₃)₂ |
| B-L-EC0611 | —O—(CH₂)₂CH₃ |
| B-L-EC0623 | —S—(CH₂)₂CH₃ |
| B-L-EC0649 | —S—CH₂CH₃ |
| B-L-EC0650 | —S—(CH₂)₄CH₃ |

and pharmaceutical salts thereof.

As described herein, the tubulysin compounds may be inhibitors of tubulin polymerization, and also may be DNA-alkylators. Accordingly, methods for treating diseases and disease states including pathogenic cell populations, such as cancer, are contemplated herein.

In another embodiment, the bivalent linker (L) is a chain of atoms selected from C, N, O, S, Si, and P that covalently connects the binding ligand (B) to the tubulysin (D). The linker may have a wide variety of lengths, such as in the range from about 2 to about 100 atoms. The atoms used in forming the linker may be combined in all chemically relevant ways, such as chains of carbon atoms forming alkylene, alkenylene, and alkynylene groups, and the like; chains of carbon and oxygen atoms forming ethers, polyoxyalkylene groups, or when combined with carbonyl groups forming esters and carbonates, and the like; chains of carbon and nitrogen atoms forming amines, imines, polyamines, hydrazines, hydrazones, or when combined with carbonyl groups forming amides, ureas, semicarbazides, carbazides, and the like; chains of carbon, nitrogen, and oxygen atoms forming alkoxyamines, alkoxyl amines, or when combined with carbonyl groups forming urethanes, amino acids, acyloxyl amines, hydroxamic acids, and the like; and many others. In addition, it is to be understood that the atoms forming the chain in each of the foregoing illustrative embodiments may be either saturated or unsaturated, such that for example, alkanes, alkenes, alkynes, imines, and the like may be radicals that are included in the linker. In addition, it is to be understood that the atoms forming the linker may also be cyclized upon each other to form divalent cyclic structures that form the linker, including cyclo alkanes, cyclic ethers, cyclic amines, arylenes, heteroarylenes, and the like in the linker.

In another embodiment, the linker includes radicals that form at least one releasable linker, and optionally one or more spacer linkers. As used herein, the term releasable linker refers to a linker that includes at least one bond that can be broken under physiological conditions, such as a pH-labile, acid-labile, base-labile, oxidatively labile, metabolically labile, biochemically labile, or enzyme-labile bond. It is appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It is understood that a cleavable bond can connect two adjacent atoms within the releasable linker and/or connect other linkers or B and/or D, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, such as an additional heteroatom, a spacer linker, another releasable linker, the tubulysin, or analog or derivative thereof, or the binding ligand, following breakage of the bond, the releasable linker is separated from the other moiety. Accordingly, it is also understood that each of the spacer and releasable linkers are polyvalent, such as bivalent.

Illustrative releasable linkers include methylene, 1-alkoxyalkylene, 1-alkoxycycloalkylene, 1-alkoxyalkylenecarbonyl, 1-alkoxycycloalkylenecarbonyl, carbonylarylcarbonyl, carbonyl(carboxyaryl)carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio, and carbonylalkylthio, wherein each of the releasable linkers is optionally substituted with a substituent X², as defined below.

In the preceding embodiment, the releasable linker may include oxygen, and the releasable linkers can be methylene, 1-alkoxyalkylene, 1-alkoxycycloalkylene, 1-alkoxyalkylenecarbonyl, and 1-alkoxycycloalkylenecarbonyl, wherein each of the releasable linkers is optionally substituted with a substituent X², as defined below, and the releasable linker is bonded to the oxygen to form an acetal or ketal. Alternatively, the releasable linker may include oxygen, and the releasable linker can be methylene, wherein the methylene is substituted with an optionally-substituted aryl, and the releasable linker is bonded to the oxygen to form an acetal or ketal. Further, the releasable linker may include oxygen, and the releasable linker can be sulfonylalkyl, and the releasable linker is bonded to the oxygen to form an alkylsulfonate.

In another embodiment of the above releasable linker embodiment, the releasable linker may include nitrogen, and the releasable linkers can be iminoalkylidenyl, carbonylalkylideniminyl, iminocycloalkylidenyl, and carbonylcycloalkylideniminyl, wherein each of the releasable linkers is optionally substituted with a substituent X², as defined below, and the releasable linker is bonded to the nitrogen to form an hydrazone. In an alternate configuration, the hydrazone may be acylated with a carboxylic acid derivative, an orthoformate derivative, or a carbamoyl derivative to form various acylhydrazone releasable linkers.

Alternatively, the releasable linker may include oxygen, and the releasable linkers can be alkylene(dialkylsilyl), alkylene(alkylarylsilyl), alkylene(diarylsilyl), (dialkylsilyl)aryl, (alkylarylsilyl)aryl, and (diarylsilyl)aryl, wherein each of the releasable linkers is optionally substituted with a substituent X², as defined below, and the releasable linker is bonded to the oxygen to form a silanol. In another variation, the tubulysin can include an oxygen atom, and the releasable linker can be haloalkylenecarbonyl, optionally substituted with a substituent X², and the releasable linker is bonded to the tubulysin oxygen to form an ester.

In the above releasable linker embodiment, the tubulysin can include a nitrogen atom, the releasable linker may include nitrogen, and the releasable linkers can be carbonylarylcarbonyl, carbonyl(carboxyaryl)carbonyl, carbonyl(biscarboxyaryl)carbonyl, and the releasable linker can be bonded to the heteroatom nitrogen to form an amide, and also bonded to the tubulysin nitrogen to form an amide. In one variation, the tubulysin can include a nitrogen atom, and the releasable linker can be haloalkylenecarbonyl, optionally substituted with a substituent X², and the releasable linker is bonded to the tubulysin nitrogen to form an amide. In another variation, the tubulysin can include a double-bonded nitrogen atom, and in this embodiment, the releasable linkers can be alkylenecarbonylamino and 1-(alkylenecarbonylamino)succinimid-3-yl, and the releasable linker can be bonded to the tubulysin nitrogen to form an hydrazone.

In another variation, the tubulysin can include a sulfur atom, and in this embodiment, the releasable linkers can be alkylenethio and carbonylalkylthio, and the releasable linker can be bonded to the tubulysin sulfur to form a disulfide. Alternatively, the tubulysin can include an oxygen atom, the releasable linker may include nitrogen, and the releasable linkers can be carbonylarylcarbonyl, carbonyl(carboxyaryl)carbonyl, carbonyl(biscarboxyaryl)carbonyl, and the releasable linker can form an amide, and also bonded to the tubulysin oxygen to form an ester.

The substituents X² can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment the releasable linker can include nitrogen, and the substituent X² and the releasable linker can form an heterocycle.

The heterocycles can be pyrrolidines, piperidines, oxazolidines, isoxazolidines, thiazolidines, isothiazolidines, pyrrolidinones, piperidinones, oxazolidinones, isoxazolidinones, thiazolidinones, isothiazolidinones, and succinimides.

In another embodiment, the bivalent linker (L) includes a disulfide releasable linker. In another embodiment, the bivalent linker (L) includes at least one releasable linker that is not a disulfide releasable linker.

In one aspect, the releasable and spacer linkers may be arranged in such a way that subsequent to the cleavage of a bond in the bivalent linker, released functional groups chemically assist the breakage or cleavage of additional bonds, also termed anchimeric assisted cleavage or breakage. An illustrative embodiment of such a bivalent linker or portion thereof includes compounds having the formulae:

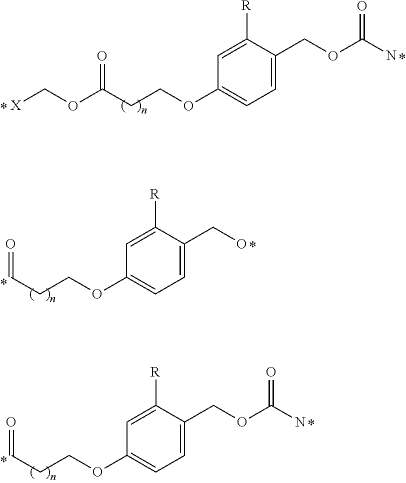

where X is an heteroatom, such as nitrogen, oxygen, or sulfur, or a carbonyl group; n is an integer selected from 0 to 4; illustratively 2; R is hydrogen, or a substituent, including a substituent capable of stabilizing a positive charge inductively or by resonance on the aryl ring, such as alkoxy and the like, including methoxy; and the symbol (*) indicates points of attachment for additional spacer, heteroatom, or releasable linkers forming the bivalent linker, or alternatively for attachment of the tubulysin, or analog or derivative thereof, or the folate, or analog or derivative thereof. In one embodiment, n is 2 and R is methoxy. It is appreciated that other substituents may be present on the aryl ring, the benzyl carbon, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimerically assisted mechanism.

Illustrative examples of intermediates useful in forming such linkers include:

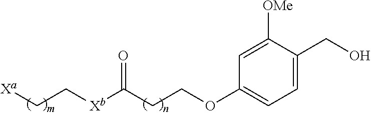

where X^{a} is an electrophilic group such as maleimide, vinyl sulfone, activated carboxylic acid derivatives, and the like, X^{b} is NH, O, or S; and m and n are each independently selected integers from 0-4. In one variation, m and n are each independently selected integers from 0-2. Such intermediates may be coupled to tubulysins, binding ligands, or other linkers via nucleophilic attack onto electrophilic group X^{a}, and/or by forming ethers or carboxylic acid derivatives of the. In one embodiment, the benzylic hydroxyl group is converted into the corresponding activated benzyloxycarbonyl compound with phosgene or a phosgene equivalent. This embodiment may be coupled to tubulysins, binding ligands, or other linkers via nucleophilic attack onto the activated carbonyl group.

The releasable linker includes at least one bond that can be broken or cleaved under physiological conditions (e.g., a pH-labile, acid-labile, oxidatively-labile, or enzyme-labile bond). The cleavable bond or bonds may be present in the interior of a cleavable linker and/or at one or both ends of a cleavable linker. It is appreciated that the lability of the cleavable bond may be adjusted by including functional groups or fragments within the bivalent linker L that are able to assist or facilitate such bond breakage, also termed anchimeric assistance. In addition, it is appreciated that additional functional groups or fragments may be included within the bivalent linker L that are able to assist or facilitate additional fragmentation of the conjugates after bond breaking of the releasable linker.

The lability of the cleavable bond can be adjusted by, for example, substitutional changes at or near the cleavable bond, such as including alpha branching adjacent to a cleavable disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like.

Illustrative mechanisms for cleavage of the bivalent linkers described herein include the following 1,4 and 1,6 fragmentation mechanisms

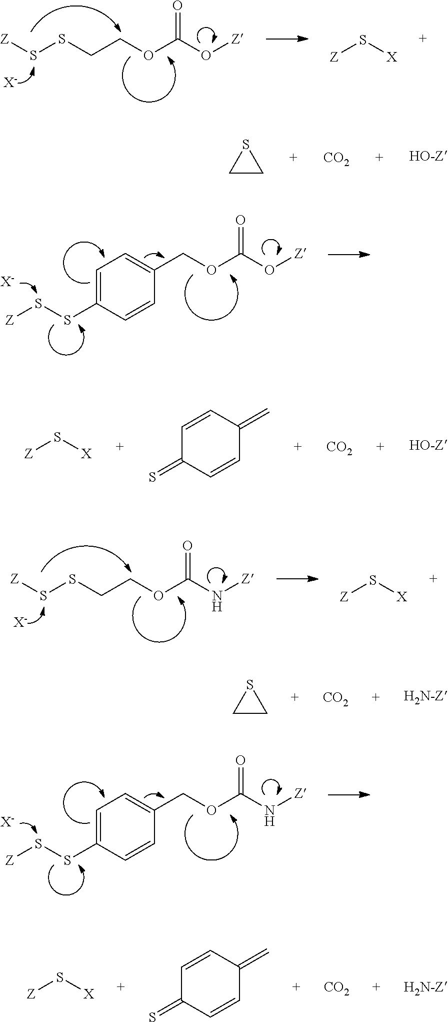

where X is an exogenous or endogenous nucleophile, glutathione, or bioreducing agent, and the like, and either of Z or Z′ is the folate, or analog or derivative thereof, or the tubulysin, or analog or derivative thereof, or a vitamin or tubulysin moiety in conjunction with other portions of the polyvalent linker. It is to be understood that although the above fragmentation mechanisms are depicted as concerted mechanisms, any number of discrete steps may take place to effect the ultimate fragmentation of the polyvalent linker to the final products shown. For example, it is appreciated that the bond cleavage may also occur by acid-catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation may be initiated by a nucleophilic attack on the disulfide group, causing cleavage to form a thiolate. The thiolate may intermolecularly displace a carbonic acid or carbamic acid moiety and form the corresponding thiacyclopropane. In the case of the benzyl-containing polyvalent linkers, following an illustrative breaking of the disulfide bond, the resulting phenyl thiolate may further fragment to release a carbonic acid or carbamic acid moiety by forming a resonance stabilized intermediate. In any of these cases, the releasable nature of the illustrative polyvalent linkers described herein may be realized by whatever mechanism may be relevant to the chemical, metabolic, physiological, or biological conditions present.

Other illustrative mechanisms for bond cleavage of the releasable linker include oxonium-assisted cleavage as follows:

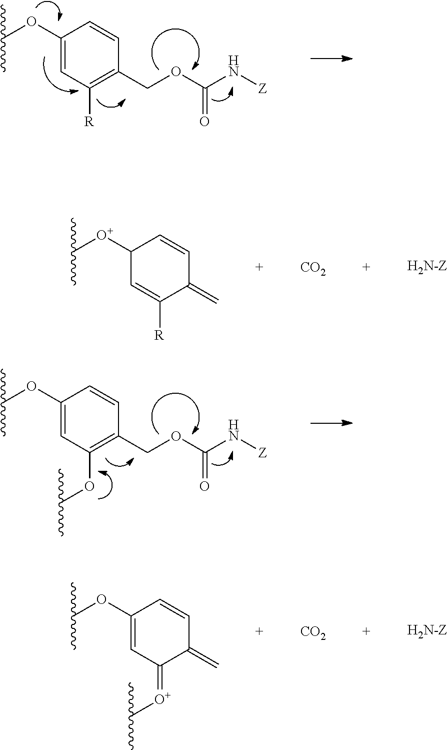

where Z is the vitamin, or analog or derivative thereof, or the tubulysin, or analog or derivative thereof, or each is a vitamin or tubulysin moiety in conjunction with other portions of the polyvalent linker, such as a tubulysin or vitamin moiety including one or more spacer linkers and/or other releasable linkers. Without being bound by theory, in this embodiment, acid catalysis, such as in an endosome, may initiate the cleavage via protonation of the urethane group. In addition, acid-catalyzed elimination of the carbamate leads to the release of CO₂ and the nitrogen-containing moiety attached to Z, and the formation of a benzyl cation, which may be trapped by water, or any other Lewis base.

Other illustrative linkers include compounds of the formulae:

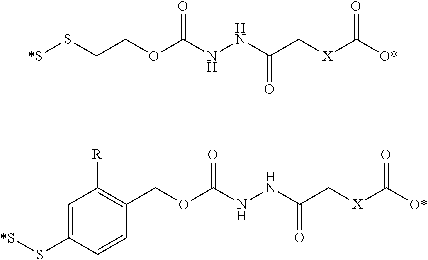

where X is NH, CH₂, or O; R is hydrogen, or a substituent, including a substituent capable of stabilizing a positive charge inductively or by resonance on the aryl ring, such as alkoxy and the like, including methoxy; and the symbol (*) indicates points of attachment for additional spacer, heteroatom, or releasable linkers forming the bivalent linker, or alternatively for attachment of the tubulysin, or analog or derivative thereof, or the vitamin, or analog or derivative thereof.

Illustrative mechanisms for cleavage of such bivalent linkers described herein include the following 1,4 and 1,6 fragmentation mechanisms followed by anchimerically assisted cleavage of the acylated Z′ via cyclization by the hydrazide group:

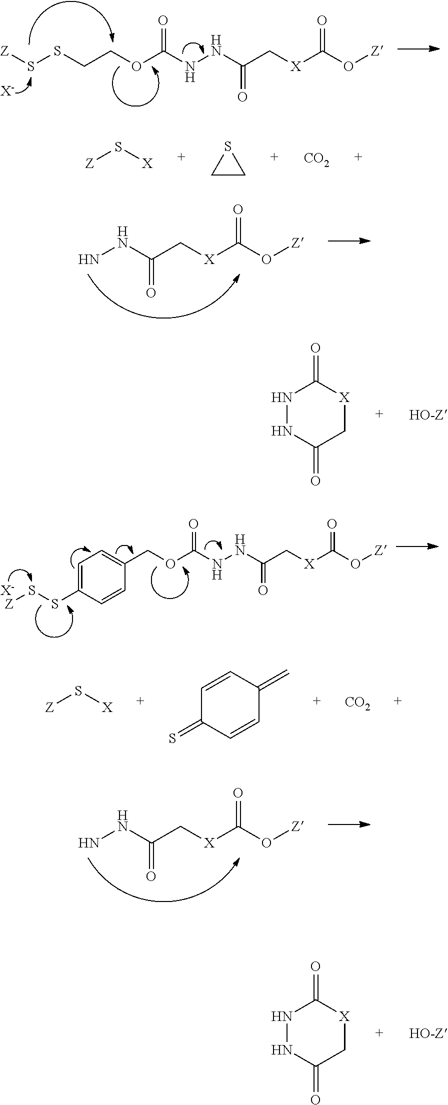

where X is an exogenous or endogenous nucleophile, glutathione, or bioreducing agent, and the like, and either of Z or Z′ is the vitamin, or analog or derivative thereof, or the tubulysin, or analog or derivative thereof, or a vitamin or tubulysin moiety in conjunction with other portions of the polyvalent linker. It is to be understood that although the above fragmentation mechanisms are depicted as concerted mechanisms, any number of discrete steps may take place to effect the ultimate fragmentation of the polyvalent linker to the final products shown. For example, it is appreciated that the bond cleavage may also occur by acid-catalyzed elimination of the carbamate moiety, which may be anchimerically assisted by the stabilization provided by either the aryl group of the beta sulfur or disulfide illustrated in the above examples. In those variations of this embodiment, the releasable linker is the carbamate moiety. Alternatively, the fragmentation may be initiated by a nucleophilic attack on the disulfide group, causing cleavage to form a thiolate. The thiolate may intermolecularly displace a carbonic acid or carbamic acid moiety and form the corresponding thiacyclopropane. In the case of the benzyl-containing polyvalent linkers, following an illustrative breaking of the disulfide bond, the resulting phenyl thiolate may further fragment to release a carbonic acid or carbamic acid moiety by forming a resonance stabilized intermediate. In any of these cases, the releasable nature of the illustrative polyvalent linkers described herein may be realized by whatever mechanism may be relevant to the chemical, metabolic, physiological, or biological conditions present. Without being bound by theory, in this embodiment, acid catalysis, such as in an endosome, may also initiate the cleavage via protonation of the urethane group. In addition, acid-catalyzed elimination of the carbamate leads to the release of CO₂ and the nitrogen-containing moiety attached to Z, and the formation of a benzyl cation, which may be trapped by water, or any other Lewis base, as is similarly described herein.

In one embodiment, the polyvalent linkers described herein are compounds of the following formulae

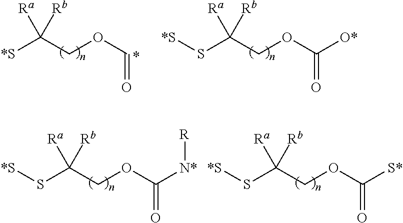

where n is an integer selected from 1 to about 4; R^{a} and R^{b} are each independently selected from the group consisting of hydrogen and alkyl, including lower alkyl such as C₁-C₄ alkyl that are optionally branched; or R^{a} and R^{b} are taken together with the attached carbon atom to form a carbocyclic ring; R is an optionally substituted alkyl group, an optionally substituted acyl group, or a suitably selected nitrogen protecting group; and (*) indicates points of attachment for the tubulysin or folate, other polyvalent linkers, or other parts of the conjugate.

In another embodiment, the polyvalent linkers described herein include compounds of the following formulae

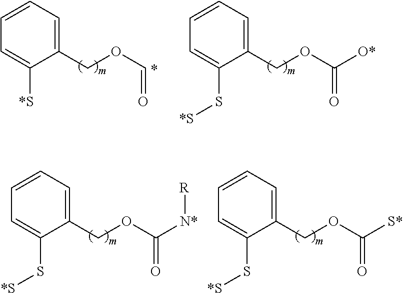

where m is an integer selected from 1 to about 4; R is an optionally substituted alkyl group, an optionally substituted acyl group, or a suitably selected nitrogen protecting group; and (*) indicates points of attachment for the tubulysin, folate, other polyvalent linkers, or other parts of the conjugate.

In another embodiment, the polyvalent linkers described herein include compounds of the following formulae

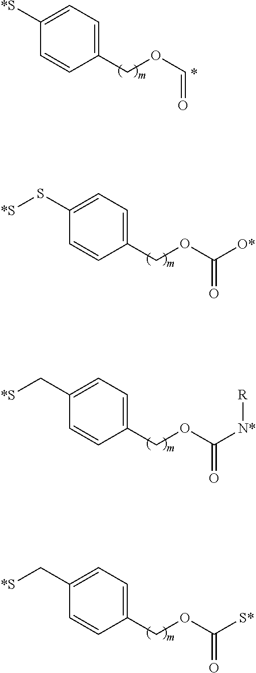

where m is an integer selected from 1 to about 4; R is an optionally substituted alkyl group, an optionally substituted acyl group, or a suitably selected nitrogen protecting group; and (*) indicates points of attachment for the tubulysin, folate, other polyvalent linkers, or other parts of the conjugate.

Another illustrative mechanism involves an arrangement of the releasable and spacer linkers in such a way that subsequent to the cleavage of a bond in the bivalent linker, released functional groups chemically assist the breakage or cleavage of additional bonds, also termed anchimeric assisted cleavage or breakage. An illustrative embodiment of such a bivalent linker or portion thereof includes compounds having the formula:

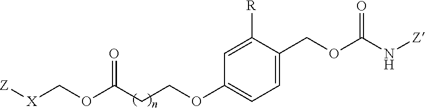

where X is an heteroatom, such as nitrogen, oxygen, or sulfur, n is an integer selected from 0, 1, 2, and 3, R is hydrogen, or a substituent, including a substituent capable of stabilizing a positive charge inductively or by resonance on the aryl ring, such as alkoxy, and the like, and either of Z or Z′ is the vitamin, or analog or derivative thereof, or the tubulysin, or analog or derivative thereof, or a vitamin or tubulysin moiety in conjunction with other portions of the bivalent linker. It is appreciated that other substituents may be present on the aryl ring, the benzyl carbon, the carbamate nitrogen, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimerically assisted mechanism.

In this embodiment, the hydroxyalkanoic acid, which may cyclize, facilitates cleavage of the methylene bridge, by for example an oxonium ion, and facilitates bond cleavage or subsequent fragmentation after bond cleavage of the releasable linker. Alternatively, acid catalyzed oxonium ion-assisted cleavage of the methylene bridge may begin a cascade of fragmentation of this illustrative bivalent linker, or fragment thereof. Alternatively, acid-catalyzed hydrolysis of the carbamate may facilitate the beta elimination of the hydroxyalkanoic acid, which may cyclize, and facilitate cleavage of methylene bridge, by for example an oxonium ion. It is appreciated that other chemical mechanisms of bond breakage or cleavage under the metabolic, physiological, or cellular conditions described herein may initiate such a cascade of fragmentation. It is appreciated that other chemical mechanisms of bond breakage or cleavage under the metabolic, physiological, or cellular conditions described herein may initiate such a cascade of fragmentation.

In another embodiment, the releasable and spacer linkers may be arranged in such a way that subsequent to the cleavage of a bond in the polyvalent linker, released functional groups chemically assist the breakage or cleavage of additional bonds, also termed anchimeric assisted cleavage or breakage. An illustrative embodiment of such a polyvalent linker or portion thereof includes compounds having the formula:

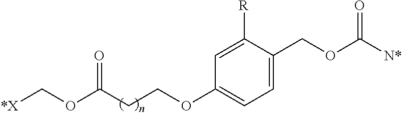

where X is an heteroatom, such as nitrogen, oxygen, or sulfur, n is an integer selected from 0, 1, 2, and 3, R is hydrogen, or a substituent, including a substituent capable of stabilizing a positive charge inductively or by resonance on the aryl ring, such as alkoxy, and the like, and the symbol (*) indicates points of attachment for additional spacer, heteroatom, or releasable linkers forming the polyvalent linker, or alternatively for attachment of the tubulysin, or analog or derivative thereof, or the folate, or analog or derivative thereof. It is appreciated that other substituents may be present on the aryl ring, the benzyl carbon, the alkanoic acid, or the methylene bridge, including but not limited to hydroxy, alkyl, alkoxy, alkylthio, halo, and the like. Assisted cleavage may include mechanisms involving benzylium intermediates, benzyne intermediates, lactone cyclization, oxonium intermediates, beta-elimination, and the like. It is further appreciated that, in addition to fragmentation subsequent to cleavage of the releasable linker, the initial cleavage of the releasable linker may be facilitated by an anchimerically assisted mechanism.

Another illustrative embodiment of the linkers described herein, include releasable linkers that cleave under the conditions described herein by a chemical mechanism involving beta elimination. In one aspect, such releasable linkers include beta-thio, beta-hydroxy, and beta-amino substituted carboxylic acids and derivatives thereof, such as esters, amides, carbonates, carbamates, and ureas. In another aspect, such releasable linkers include 2- and 4-thioarylesters, carbamates, and carbonates.

In another illustrative embodiment, the linker includes one or more amino acids. In one variation, the linker includes a single amino acid. In another variation, the linker includes a peptide having from 2 to about 50, 2 to about 30, or 2 to about 20 amino acids. In another variation, the linker includes a peptide having from about 4 to about 8 amino acids. Such amino acids are illustratively selected from the naturally occurring amino acids, or stereoisomers thereof. The amino acid may also be any other amino acid, such as any amino acid having the general formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In one variation, the releasable linker includes at least 2 amino acids selected from asparagine, aspartic acid, cysteine, glutamic acid, lysine, glutamine, arginine, serine, ornithine, and threonine. In another variation, the releasable linker includes between 2 and about 5 amino acids selected from asparagine, aspartic acid, cysteine, glutamic acid, lysine, glutamine, arginine, serine, ornithine, and threonine. In another variation, the releasable linker includes a tripeptide, tetrapeptide, pentapeptide, or hexapeptide consisting of amino acids selected from aspartic acid, cysteine, glutamic acid, lysine, arginine, and ornithine, and combinations thereof.

In another illustrative aspect of the conjugate intermediate described herein, the tubulysin, or an analog or a derivative thereof, includes an alkylthiol nucleophile.

In another embodiment, the spacer linker can be 1-alkylenesuccinimid-3-yl, optionally substituted with a substituent X¹, as defined below, and the releasable linkers can be methylene, 1-alkoxyalkylene, 1-alkoxycycloalkylene, 1-alkoxyalkylenecarbonyl, 1-alkoxycycloalkylenecarbonyl, wherein each of the releasable linkers is optionally substituted with a substituent X², as defined below, and wherein the spacer linker and the releasable linker are each bonded to the spacer linker to form a succinimid-1-ylalkyl acetal or ketal.

The spacer linkers can be carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, wherein each of the spacer linkers is optionally substituted with a substituent X¹, as defined below. In this embodiment, the spacer linker may include an additional nitrogen, and the spacer linkers can be alkylenecarbonyl, cycloalkylenecarbonyl, carbonylalkylcarbonyl, 1-(carbonylalkyl)succinimid-3-yl, wherein each of the spacer linkers is optionally substituted with a substituent X¹, as defined below, and the spacer linker is bonded to the nitrogen to form an amide. Alternatively, the spacer linker may include an additional sulfur, and the spacer linkers can be alkylene and cycloalkylene, wherein each of the spacer linkers is optionally substituted with carboxy, and the spacer linker is bonded to the sulfur to form a thiol. In another embodiment, the spacer linker can include sulfur, and the spacer linkers can be 1-alkylenesuccinimid-3-yl and 1-(carbonylalkyl)succinimid-3-yl, and the spacer linker is bonded to the sulfur to form a succinimid-3-ylthiol.

In an alternative to the above-described embodiments, the spacer linker can include nitrogen, and the releasable linker can be a divalent radical comprising alkyleneaziridin-1-yl, carbonylalkylaziridin-1-yl, sulfoxylalkylaziridin-1-yl, or sulfonylalkylaziridin-1-yl, wherein each of the releasable linkers is optionally substituted with a substituent X², as defined below. In this alternative embodiment, the spacer linkers can be carbonyl, thionocarbonyl, alkylenecarbonyl, cycloalkylenecarbonyl, carbonylalkylcarbonyl, 1-(carbonylalkyl)succinimid-3-yl, wherein each of the spacer linkers is optionally substituted with a substituent X¹, as defined below, and wherein the spacer linker is bonded to the releasable linker to form an aziridine amide.

The substituents X¹ can be alkyl, alkoxy, alkoxyalkyl, hydroxy, hydroxyalkyl, amino, aminoalkyl, alkylaminoalkyl, dialkylaminoalkyl, halo, haloalkyl, sulfhydrylalkyl, alkylthioalkyl, aryl, substituted aryl, arylalkyl, substituted arylalkyl, heteroaryl, substituted heteroaryl, carboxy, carboxyalkyl, alkyl carboxylate, alkyl alkanoate, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides. In this embodiment the spacer linker can include nitrogen, and the substituent X¹ and the spacer linker to which they are bound to form an heterocycle.

In one aspect of the various conjugates described herein, the bivalent linker comprises a spacer linker and a releasable linker taken together to form 3-thiosuccinimid-1-ylalkyloxymethyloxy, where the methyl is optionally substituted with alkyl or substituted aryl.

In another aspect, the bivalent linker comprises a spacer linker and a releasable linker taken together to form 3-thiosuccinimid-1-ylalkylcarbonyl, where the carbonyl forms an acylaziridine with the tubulysin, or analog or derivative thereof.

In another aspect, the bivalent linker comprises an a spacer linker and a releasable linker taken together to form 1-alkoxycycloalkylenoxy.

In another aspect, the bivalent linker comprises a spacer linker and a releasable linker taken together to form alkyleneaminocarbonyl(dicarboxylarylene)carboxylate.

In another aspect, the bivalent linker comprises a releasable linker, a spacer linker, and a releasable linker taken together to form 2- or 3-dithioalkylcarbonylhydrazide, where the hydrazide forms an hydrazone with the tubulysin, or analog or derivative thereof.

In another aspect, the bivalent linker comprises a spacer linker and a releasable linker taken together to form 3-thiosuccinimid-1-ylalkylcarbonylhydrazide, where the hydrazide forms an hydrazone with the tubulysin, or analog or derivative thereof.

In another aspect, the bivalent linker comprises a spacer linker and a releasable linker taken together to form 2- or 3-thioalkylsulfonylalkyl(disubstituted silyl)oxy, where the disubstituted silyl is substituted with alkyl or optionally substituted aryl.

In another aspect, the bivalent linker comprises a plurality of spacer linkers selected from the group consisting of the naturally occurring amino acids and stereoisomers thereof.

In another aspect, the bivalent linker comprises a releasable linker, a spacer linker, and a releasable linker taken together to form 3-dithioalkyloxycarbonyl, where the carbonyl forms a carbonate with the tubulysin, or analog or derivative thereof.

In another aspect, the bivalent linker comprises a releasable linker, a spacer linker, and a releasable linker taken together to form 3-dithioarylalkyloxycarbonyl, where the carbonyl forms a carbonate with the tubulysin, or analog or derivative thereof, and the aryl is optionally substituted.

In another aspect, the bivalent linker comprises a spacer linker and a releasable linker taken together to form 3-thiosuccinimid-1-ylalkyloxyalkyloxyalkylidene, where the alkylidene forms an hydrazone with the tubulysin, or analog or derivative thereof, each alkyl is independently selected, and the oxyalkyloxy is optionally substituted with alkyl or optionally substituted aryl.

In another aspect, the bivalent linker comprises a releasable linker, a spacer linker, and a releasable linker taken together to form 2- or 3-dithioalkyloxycarbonylhydrazide.

In another aspect, the bivalent linker comprises a releasable linker, a spacer linker, and a releasable linker taken together to form 2- or 3-dithioalkylamino, where the amino forms a vinylogous amide with the tubulysin, or analog or derivative thereof.

In another aspect, the bivalent linker comprises a releasable linker, a spacer linker, and a releasable linker taken together to form 2- or 3-dithioalkylamino, where the amino forms a vinylogous amide with the tubulysin, or analog or derivative thereof, and the alkyl is ethyl.

In another aspect, the bivalent linker comprises a releasable linker, a spacer linker, and a releasable linker taken together to form 2- or 3-dithioalkylaminocarbonyl, where the carbonyl forms a carbamate with the tubulysin, or analog or derivative thereof.

In another aspect, the bivalent linker comprises a releasable linker, a spacer linker, and a releasable linker taken together to form 2- or 3-dithioalkylaminocarbonyl, where the carbonyl forms a carbamate with the tubulysin, or analog or derivative thereof, and the alkyl is ethyl.

In another aspect, the bivalent linker comprises a releasable linker, a spacer linker, and a releasable linker taken together to form 2- or 3-dithioarylalkyloxycarbonyl, where the carbonyl forms a carbamate or a carbamoylaziridine with the tubulysin, or analog or derivative thereof.

In another embodiment, the polyvalent linker includes spacer linkers and releasable linkers connected to form a polyvalent 3-thiosuccinimid-1-ylalkyloxymethyloxy group, illustrated by the following formula

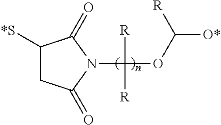

where n is an integer from 1 to 6, the alkyl group is optionally substituted, and the methyl is optionally substituted with an additional alkyl or optionally substituted aryl group, each of which is represented by an independently selected group R. The (*) symbols indicate points of attachment of the polyvalent linker fragment to other parts of the conjugates described herein.

In another embodiment, the polyvalent linker includes spacer linkers and releasable linkers connected to form a polyvalent 3-thiosuccinimid-1-ylalkylcarbonyl group, illustrated by the following formula

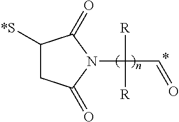

where n is an integer from 1 to 6, and the alkyl group is optionally substituted. The (*) symbols indicate points of attachment of the polyvalent linker fragment to other parts of the conjugates described herein. In another embodiment, the polyvalent linker includes spacer linkers and releasable linkers connected to form a polyvalent 3-thioalkylsulfonylalkyl(disubstituted silyl)oxy group, where the disubstituted silyl is substituted with alkyl and/or optionally substituted aryl groups.

In another embodiment, the polyvalent linker includes spacer linkers and releasable linkers connected to form a polyvalent dithioalkylcarbonylhydrazide group, or a polyvalent 3-thiosuccinimid-1-ylalkylcarbonylhydrazide, illustrated by the following formulae

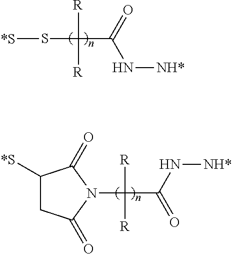

where n is an integer from 1 to 6, the alkyl group is optionally substituted, and the hydrazide forms an hydrazone with (B), (D), or another part of the polyvalent linker (L). The (*) symbols indicate points of attachment of the polyvalent linker fragment to other parts of the conjugates described herein.

In another embodiment, the polyvalent linker includes spacer linkers and releasable linkers connected to form a polyvalent 3-thiosuccinimid-1-ylalkyloxyalkyloxyalkylidene group, illustrated by the following formula

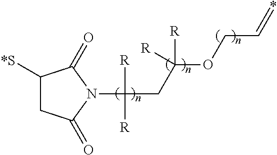

where each n is an independently selected integer from 1 to 6, each alkyl group independently selected and is optionally substituted, such as with alkyl or optionally substituted aryl, and where the alkylidene forms an hydrazone with (B), (D), or another part of the polyvalent linker (L). The (*) symbols indicate points of attachment of the polyvalent linker fragment to other parts of the conjugates described herein.

Additional illustrative spacer linkers include alkylene-amino-alkylenecarbonyl, alkylene-thio-carbonylalkylsuccinimid-3-yl, and the like, as further illustrated by the following formulae:

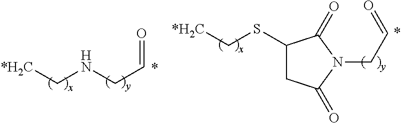

where the integers x and y are 1, 2, 3, 4, or 5:

The term cycloalkylene as used herein refers to a bivalent chain of carbon atoms, a portion of which forms a ring, such as cycloprop-1,1-diyl, cycloprop-1,2-diyl, cyclohex-1,4-diyl, 3-ethylcyclopent-1,2-diyl, 1-methylenecyclohex-4-yl, and the like.

The term heterocycle as used herein refers to a monovalent chain of carbon and heteroatoms, wherein the heteroatoms are selected from nitrogen, oxygen, and sulfur, a portion of which, including at least one heteroatom, form a ring, such as aziridine, pyrrolidine, oxazolidine, 3-methoxypyrrolidine, 3-methylpiperazine, and the like.

The term aryl as used herein refers to an aromatic mono or polycyclic ring of carbon atoms, such as phenyl, naphthyl, and the like. In addition, aryl may also include heteroaryl.

The term heteroaryl as used herein refers to an aromatic mono or polycyclic ring of carbon atoms and at least one heteroatom selected from nitrogen, oxygen, and sulfur, such as pyridinyl, pyrimidinyl, indolyl, benzoxazolyl, and the like.

The term optionally substituted as used herein refers to the replacement of one or more hydrogen atoms, generally on carbon, with a corresponding number of substituents, such as halo, hydroxy, amino, alkyl or dialkylamino, alkoxy, alkylsulfonyl, cyano, nitro, and the like. In addition, two hydrogens on the same carbon, on adjacent carbons, or nearby carbons may be replaced with a bivalent substituent to form the corresponding cyclic structure.

The term iminoalkylidenyl as used herein refers to a divalent radical containing alkylene as defined herein and a nitrogen atom, where the terminal carbon of the alkylene is double-bonded to the nitrogen atom, such as the formulae —(CH)═N—, —(CH₂)₂(CH)═N—, —CH₂C(Me)═N—, and the like.

The term amino acid as used herein refers generally to aminoalkylcarboxylate, where the alkyl radical is optionally substituted, such as with alkyl, hydroxy alkyl, sulfhydrylalkyl, aminoalkyl, carboxyalkyl, and the like, including groups corresponding to the naturally occurring amino acids, such as serine, cysteine, methionine, aspartic acid, glutamic acid, and the like. It is to be understood that such amino acids may be of a single stereochemistry or a particular mixture of stereochemistries, including racemic mixtures. In addition, amino acid refers to beta, gamma, and longer amino acids, such as amino acids of the formula:

—N(R)—(CR′R″)_{q}—C(O)—

where R is hydrogen, alkyl, acyl, or a suitable nitrogen protecting group, R′ and R″ are hydrogen or a substituent, each of which is independently selected in each occurrence, and q is an integer such as 1, 2, 3, 4, or 5. Illustratively, R′ and/or R″ independently correspond to, but are not limited to, hydrogen or the side chains present on naturally occurring amino acids, such as methyl, benzyl, hydroxymethyl, thiomethyl, carboxyl, carboxylmethyl, guanidinopropyl, and the like, and derivatives and protected derivatives thereof. The above described formula includes all stereoisomeric variations. For example, the amino acid may be selected from asparagine, aspartic acid, cysteine, glutamic acid, lysine, glutamine, arginine, serine, ornithine, threonine, and the like. In another illustrative aspect of the conjugate intermediates described herein, the tubulysin, or an analog or a derivative thereof, includes an alkylthiol nucleophile.

It is to be understood that the above-described terms can be combined to generate chemically-relevant groups, such as alkoxyalkyl referring to methyloxymethyl, ethyloxyethyl, and the like, haloalkoxyalkyl referring to trifluoromethyloxyethyl, 1,2-difluoro-2-chloroeth-1-yloxypropyl, and the like, arylalkyl referring to benzyl, phenethyl, α-methylbenzyl, and the like, and others.

The term amino acid derivative as used herein refers generally to an optionally substituted aminoalkylcarboxylate, where the amino group and/or the carboxylate group are each optionally substituted, such as with alkyl, carboxylalkyl, alkylamino, and the like, or optionally protected. In addition, the optionally substituted intervening divalent alkyl fragment may include additional groups, such as protecting groups, and the like.

The term peptide as used herein refers generally to a series of amino acids and/or amino acid analogs and derivatives covalently linked one to the other by amide bonds.

Additional linkers are described in U.S. patent application publication 2005/0002942, the disclosure of which is incorporated herein by reference, and in Tables 1 and 2 below, where the (*) atom is the point of attachment of additional spacer or releasable linkers, the tubulysin, and/or the binding ligand.

TABLE 1
Illustrative spacer linkers.

| | |
|---|---|
| 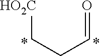 | 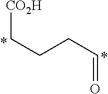 |
| 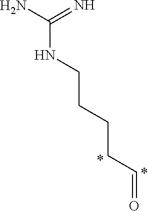 | 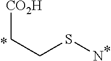 |
| 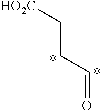 | 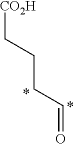 |
| 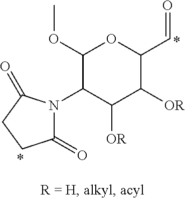 | 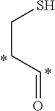 |
| 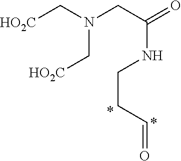 | 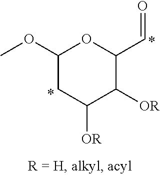 |

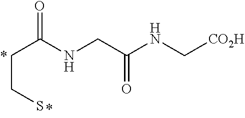 [SMILES: O=C(O)CNC(=O)CNC(=O)CCS]

| | |
|---|---|
| 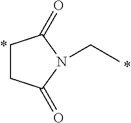 | 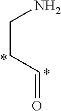 |
| 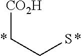 | 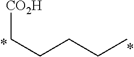 |
| 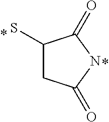 | 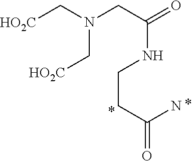 |

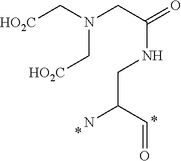 [SMILES: NC(C=O)CNC(=O)CN(CC(=O)O)CC(=O)O]

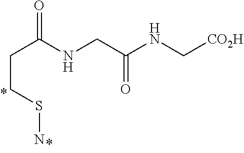 [SMILES: NSCCC(=O)NCC(=O)NCC(=O)O]

| | |
|---|---|
| 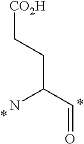 | 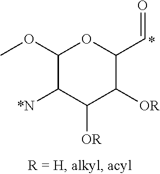 |

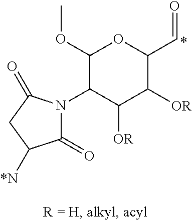

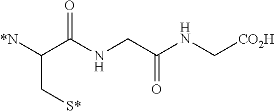

| | |
|---|---|
| 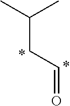 | 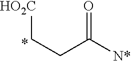 |
| 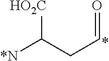 | 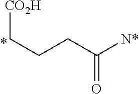 |
| 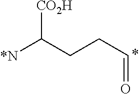 | 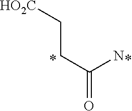 |
| 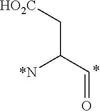 | 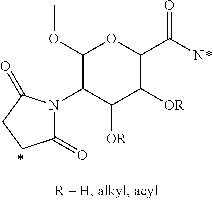 |
| 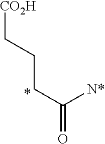 | 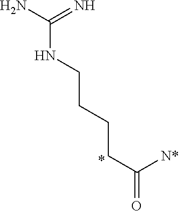 |
| 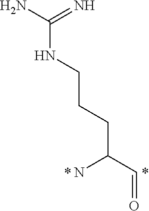 | 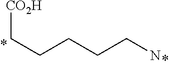 |
| 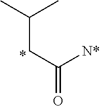 | 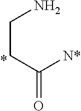 |
| 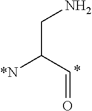 | 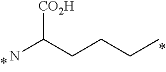 |
| 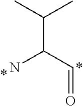 | 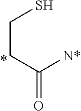 |
| 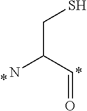 | 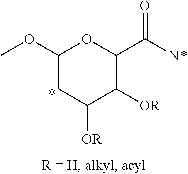 |
| 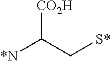 | 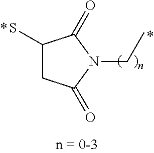 |
| 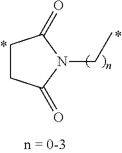 | 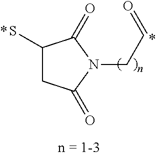 |

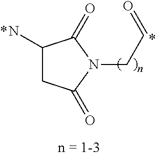

TABLE 2
Illustrative releasable linkers.

| | |
|---|---|
| 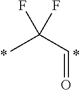 | 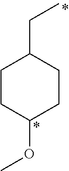 |
| 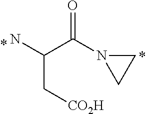 [SMILES: NC(CC(=O)O)C(=O)N1CC1] | 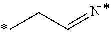 |
| 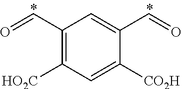 [SMILES: O=Cc1cc(C=O)c(C(=O)O)cc1C(=O)O] | 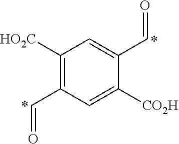 [SMILES: O=Cc1cc(C(=O)O)c(C=O)cc1C(=O)O] |
| 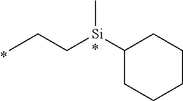 | 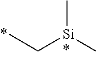 |
| 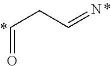 | 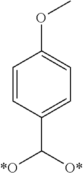 |
| 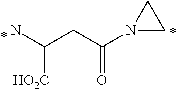 [SMILES: NC(CC(=O)N1CC1)C(=O)O] | 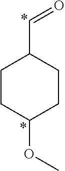 |
| 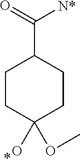 | 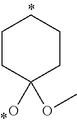 |
| 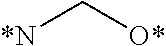 | 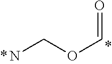 |

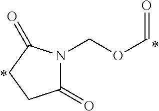 [SMILES: O=COCN1C(=O)CCC1=O]

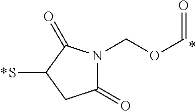 [SMILES: O=COCN1C(=O)CC(S)C1=O]

| | |
|---|---|
| 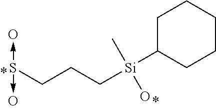 | 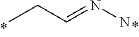 |
| 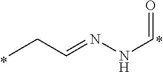 | 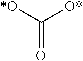 |
| 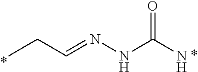 | 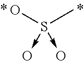 |
| 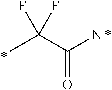 | 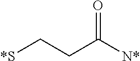 |
| 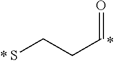 | 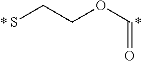 |

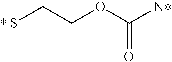

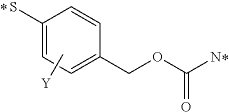

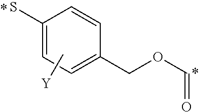

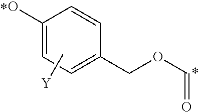

| | |
|---|---|
| 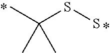 | 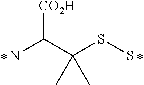 |

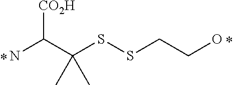

In another illustrative embodiment, bivalent linkers (L) that include spacer linkers that substantially increase the water solubility, biological transport, preferential renal clearance, uptake, absorption, biodistribution, and/or bioavailability of the conjugate are described herein. Illustrative spacer linkers that include hydrophilic groups are described, such as compounds of the formula

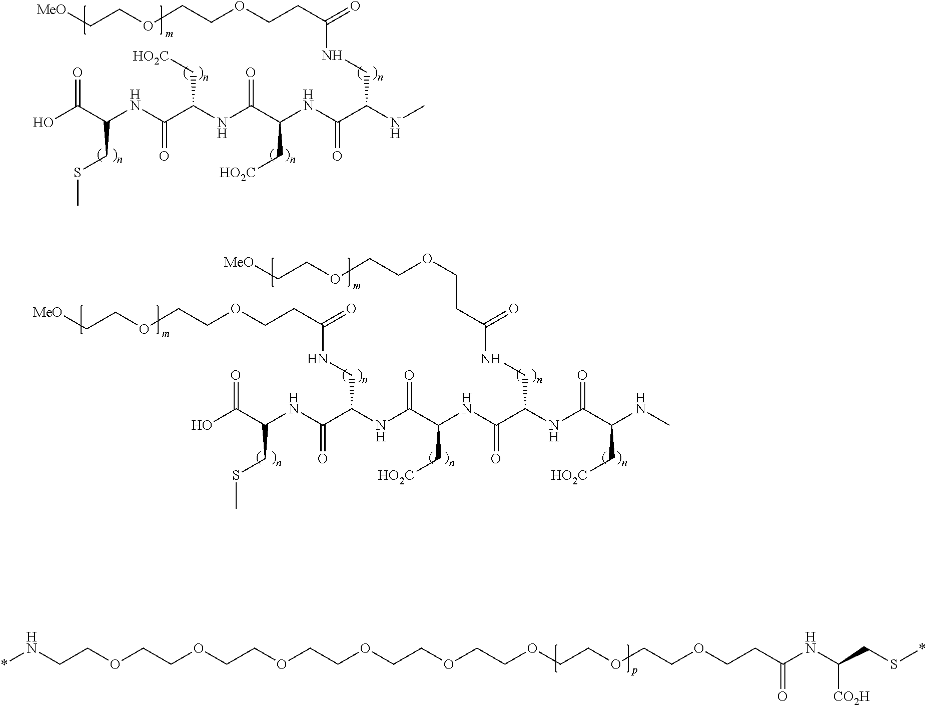

where m is an integer independently selected in each instance from 1 to about 8; p is an integer selected 1 to about 10; and n is an integer independently selected in each instance from 1 to about 3. In one aspect, m is independently in each instance 1 to about 3. In another aspect, n is 1 in each instance. In another aspect, p is independently in each instance about 4 to about 6. Illustratively, the corresponding polypropylene polyethers corresponding to the foregoing are contemplated herein and may be included in the conjugates as hydrophilic spacer linkers. In addition, it is appreciated that mixed polyethylene and polypropylene polyethers may be included in the conjugates as hydrophilic spacer linkers. Further, cyclic variations of the foregoing polyether compounds, such as those that include tetrahydrofuranyl, 1,3-dioxanes, 1,4-dioxanes, and the like are contemplated herein.

In another illustrative embodiment, the hydrophilic spacer linkers described herein include a plurality of hydroxyl functional groups, such as linkers that incorporate monosaccharides, oligosaccharides, polysaccharides, and the like. It is to be understood that the polyhydroxyl containing spacer linkers comprises a plurality of —(CROH)— groups, where R is hydrogen or alkyl.

In another embodiment, the spacer linkers include one or more of the following fragments:

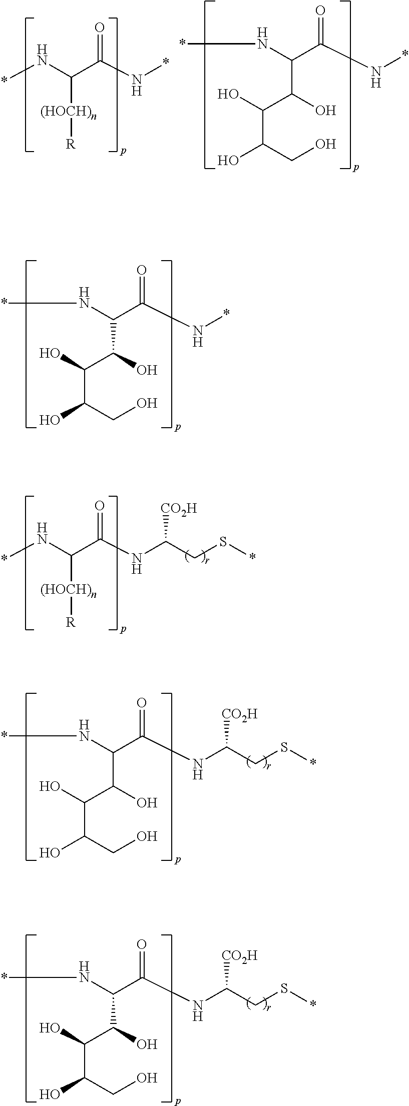

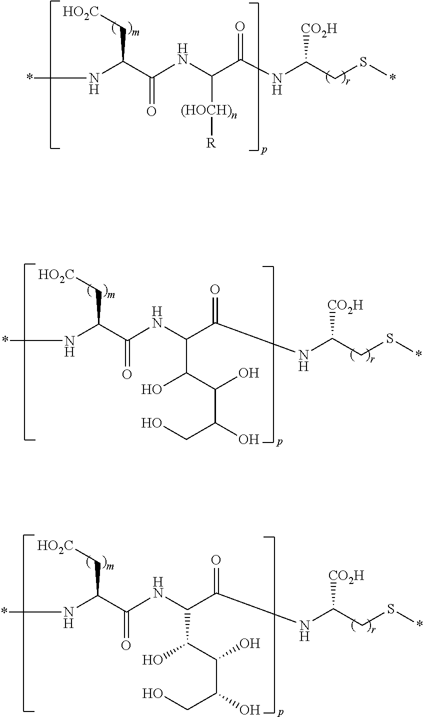

wherein R is hydrogen, alkyl, cycloalkyl, or arylalkyl; m is an integer from 1 to about 3; n is an integer from 1 to about 5, p is an integer from 1 to about 5, and r is an integer selected from 1 to about 3. In one aspect, the integer n is 3 or 4. In another aspect, the integer p is 3 or 4. In another aspect, the integer r is 1.

In another embodiment, the spacer linkers include one or more of the following fragments:

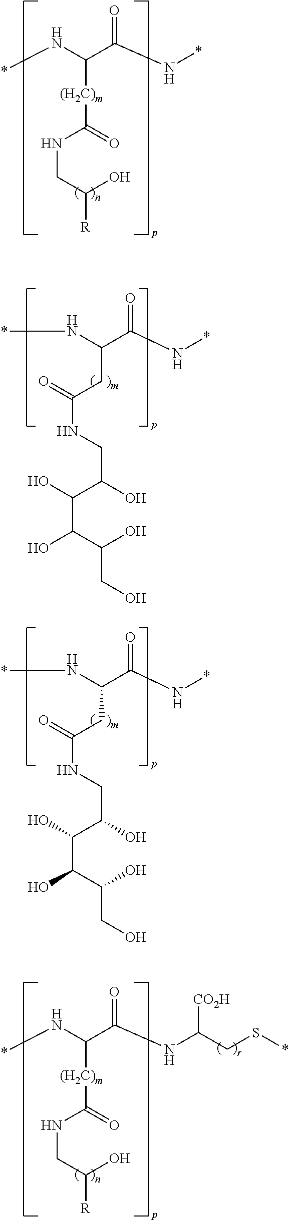

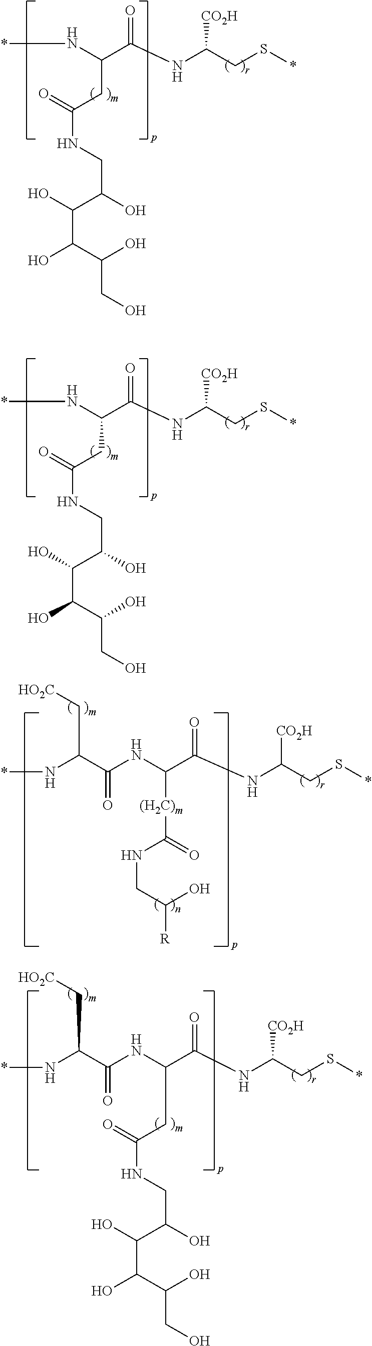

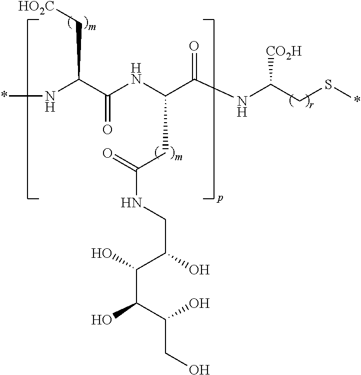

wherein R is hydrogen, alkyl, cycloalkyl, or arylalkyl; m is an independently selected integer from 1 to about 3; n is an integer from 1 to about 6, p is an integer from 1 to about 5, and r is an integer selected from 1 to about 3. In one variation, the integer n is 3 or 4. In another variation, the integer p is 3 or 4. In another variation, the integer r is 1.

In another embodiment, the spacer linker includes one or more of the following cyclic polyhydroxyl groups:

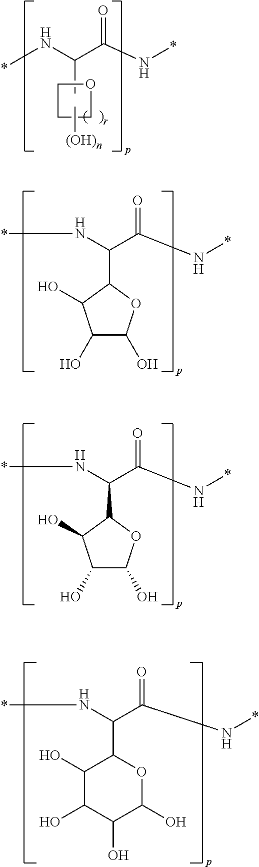

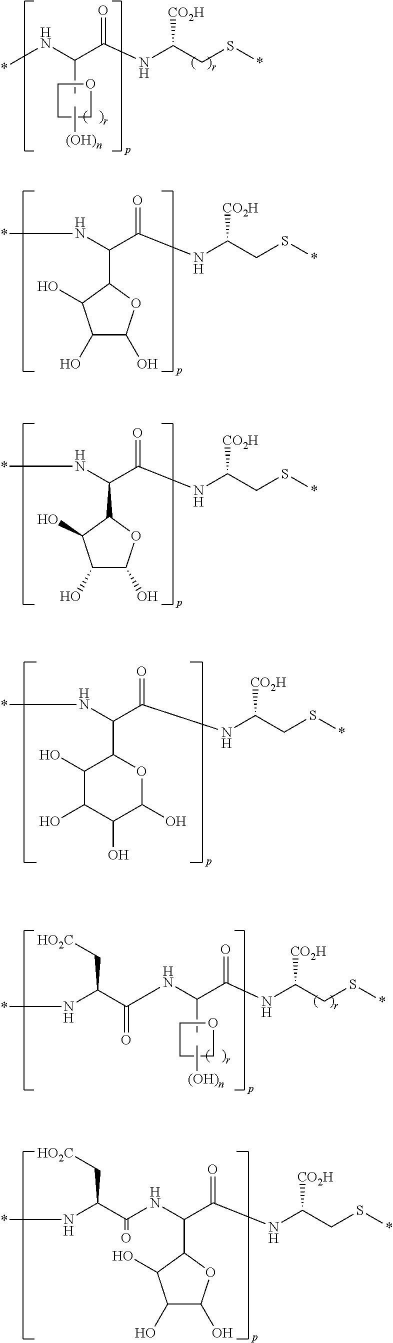

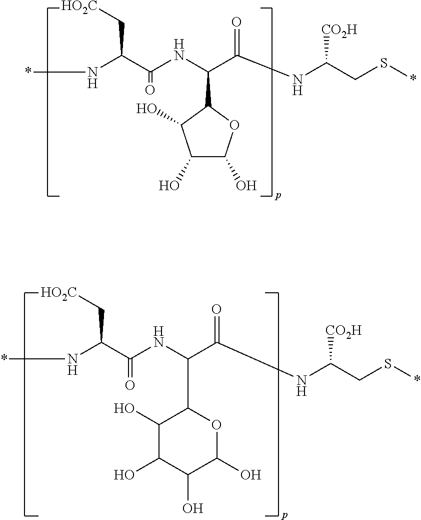

wherein n is an integer from 2 to about 5, p is an integer from 1 to about 5, and r is an integer from 1 to about 4. In one aspect, the integer n is 3 or 4. In another aspect, the integer p is 3 or 4. In another aspect, the integer r is 2 or 3. It is understood that all stereochemical forms of such sections of the linkers are contemplated herein. For example, in the above formula, the section may be derived from ribose, xylose, glucose, mannose, galactose, or other sugar and retain the stereochemical arrangements of pendant hydroxyl and alkyl groups present on those molecules. In addition, it is to be understood that in the foregoing formulae, various deoxy compounds are also contemplated. Illustratively, compounds of the following formulae are contemplated:

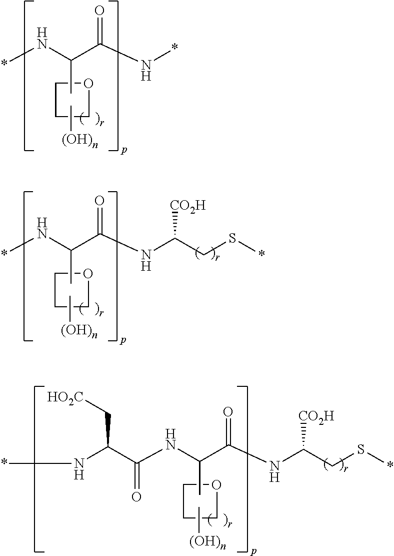

wherein n is equal to or less than r, such as when r is 2 or 3, n is 1 or 2, or 1, 2, or 3, respectively.

In another embodiment, the spacer linker includes a polyhydroxyl compound of the following formula:

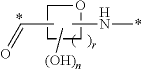

wherein n and r are each an integer selected from 1 to about 3. In one aspect, the spacer linker includes one or more polyhydroxyl compounds of the following formulae:

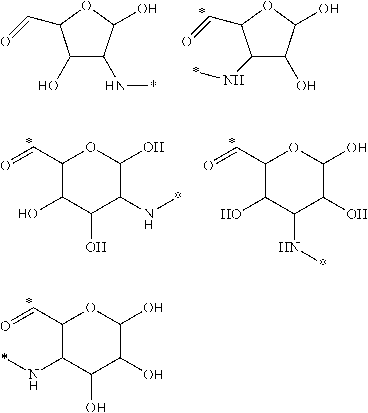

It is understood that all stereochemical forms of such sections of the linkers are contemplated herein. For example, in the above formula, the section may be derived from ribose, xylose, glucose, mannose, galactose, or other sugar and retain the stereochemical arrangements of pendant hydroxyl and alkyl groups present on those molecules.

In another configuration, the hydrophilic linkers L described herein include polyhydroxyl groups that are spaced away from the backbone of the linker. Illustratively, such linkers include fragments of the following formulae:

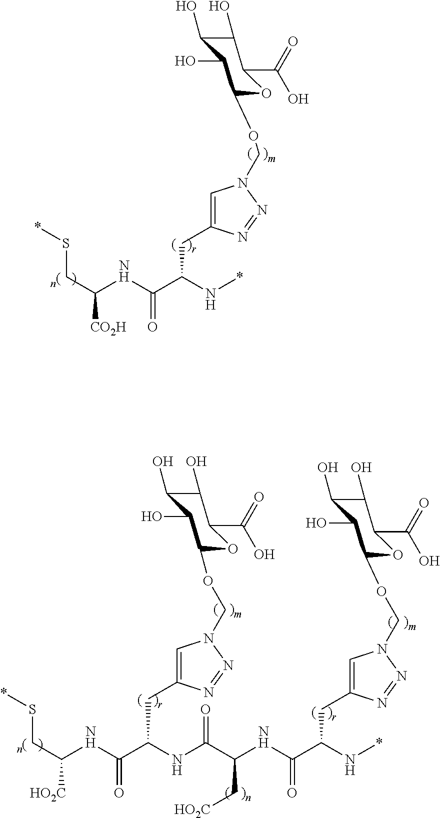

wherein n, m, and r are integers and are each independently selected in each instance from 1 to about 5. In one illustrative aspect, m is independently 2 or 3 in each instance. In another aspect, r is 1 in each instance. In another aspect, n is 1 in each instance. In one variation, the group connecting the polyhydroxyl group to the backbone of the linker is a different heteroaryl group, including but not limited to, pyrrole, pyrazole, 1,2,4-triazole, furan, oxazole, isoxazole, thienyl, thiazole, isothiazole, oxadiazole, and the like. Similarly, divalent 6-membered ring heteroaryl groups are contemplated. Other variations of the foregoing illustrative hydrophilic spacer linkers include oxyalkylene groups, such as the following formulae:

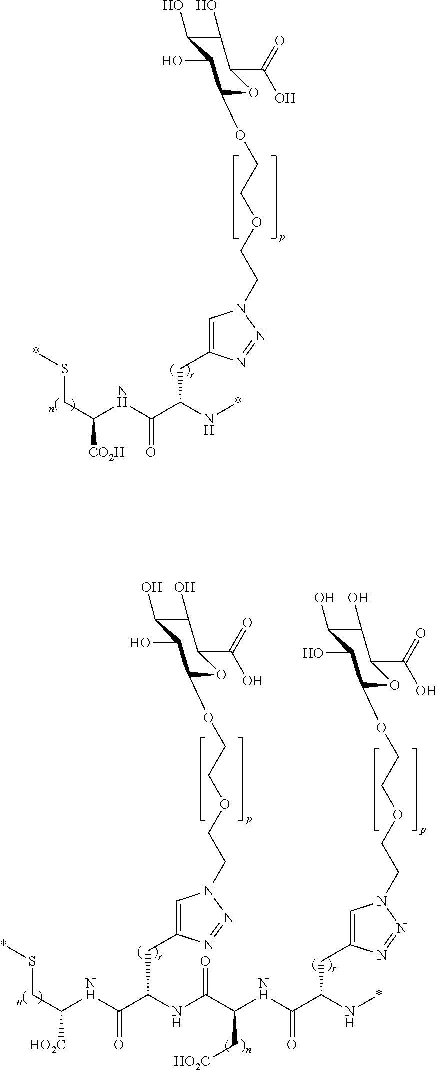

wherein n and r are integers and are each independently selected in each instance from 1 to about 5; and p is an integer selected from 1 to about 4.

In another embodiment, the hydrophilic linkers L described herein include polyhydroxyl groups that are spaced away from the backbone of the linker. Illustratively, such linkers include fragments of the following formulae:

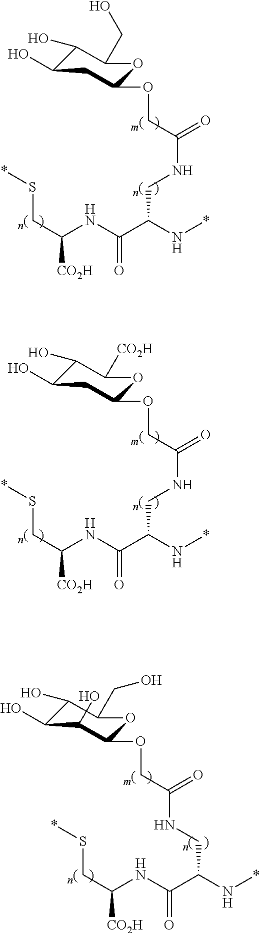

wherein n is an integer selected from 1 to about 3, and m is an integer selected from 1 to about 22. In one illustrative aspect, n is 1 or 2. In another illustrative aspect, m is selected from about 6 to about 10, illustratively 8. In one variation, the group connecting the polyhydroxyl group to the backbone of the linker is a different functional group, including but not limited to, esters, ureas, carbamates, acylhydrazones, and the like. Similarly, cyclic variations are contemplated. Other variations of the foregoing illustrative hydrophilic spacer linkers include oxyalkylene groups, such as the following formulae:

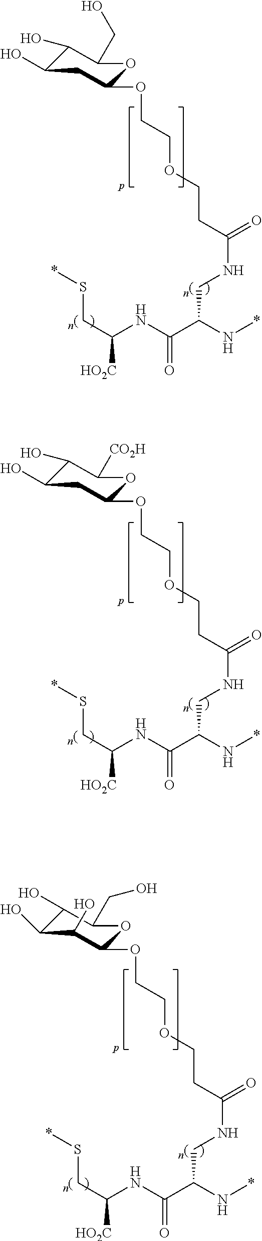

wherein n and r are integers and are each independently selected in each instance from 1 to about 5; and p is an integer selected from 1 to about 4.

In another embodiment, the hydrophilic spacer linker is a combination of backbone and branching side motifs such as is illustrated by the following formulae

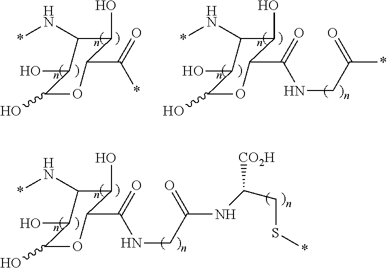

wherein n is an integer independently selected in each instance from 0 to about 3. The above formula are intended to represent 4, 5, 6, and even larger membered cyclic sugars. In addition, it is to be understood that the above formula may be modified to represent deoxy sugars, where one or more of the hydroxy groups present on the formulae are replaced by hydrogen, alkyl, or amino. In addition, it is to be understood that the corresponding carbonyl compounds are contemplated by the above formulae, where one or more of the hydroxyl groups is oxidized to the corresponding carbonyl. In addition, in this illustrative embodiment, the pyranose includes both carboxyl and amino functional groups and (a) can be inserted into the backbone and (b) can provide synthetic handles for branching side chains in variations of this embodiment. Any of the pendant hydroxyl groups may be used to attach other chemical fragments, including additional sugars to prepare the corresponding oligosaccharides. Other variations of this embodiment are also contemplated, including inserting the pyranose or other sugar into the backbone at a single carbon, i.e. a Spiro arrangement, at a geminal pair of carbons, and like arrangements. For example, one or two ends of the linker, or the agent A, or the binding ligand B may be connected to the sugar to be inserted into the backbone in a 1,1; 1,2; 1,3; 1,4; 2,3, or other arrangement.

In another embodiment, the hydrophilic spacer linkers described herein include are formed primarily from carbon, hydrogen, and nitrogen, and have a carbon/nitrogen ratio of about 3:1 or less, or of about 2:1 or less. In one aspect, the hydrophilic linkers described herein include a plurality of amino functional groups.

In another embodiment, the spacer linkers include one or more amino groups of the following formulae:

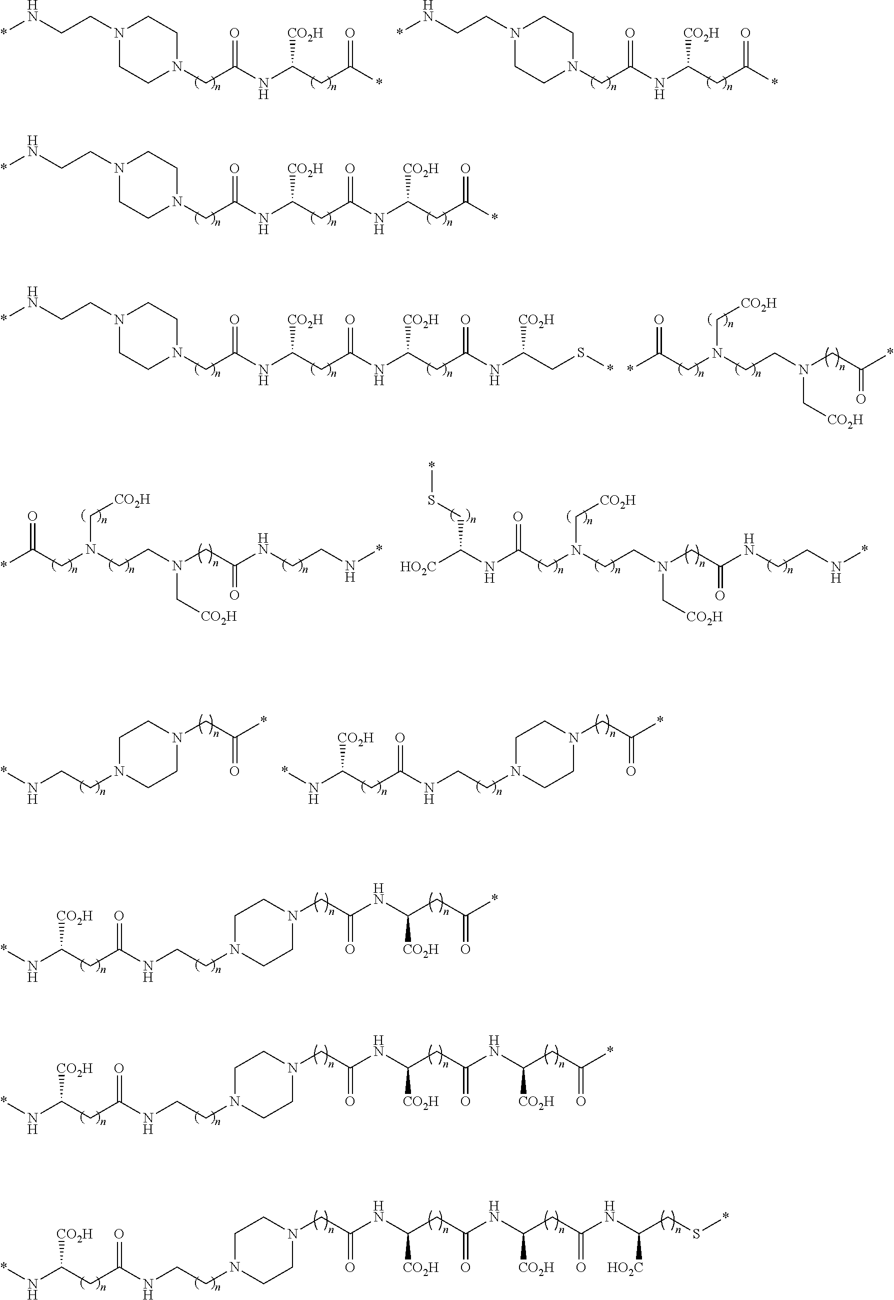

where n is an integer independently selected in each instance from 1 to about 3. In one aspect, the integer n is independently 1 or 2 in each instance. In another aspect, the integer n is 1 in each instance.

In another embodiment, the hydrophilic spacer linker is a sulfuric acid ester, such as an alkyl ester of sulfuric acid. Illustratively, the spacer linker is of the following formula:

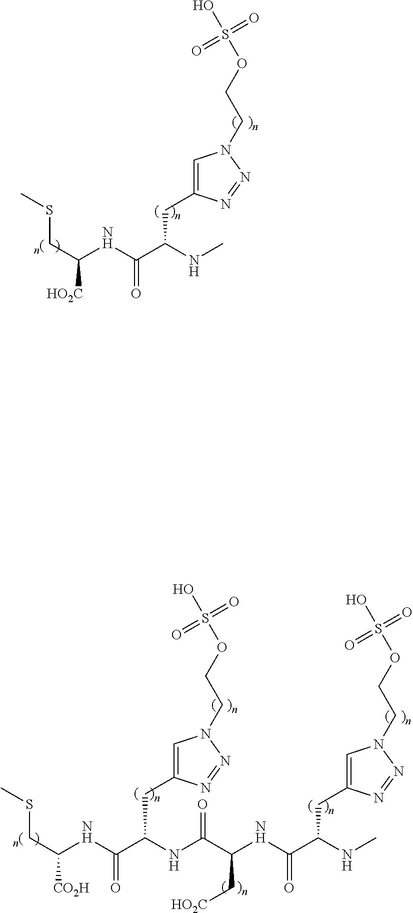

where n is an integer independently selected in each instance from 1 to about 3. Illustratively, n is independently 1 or 2 in each instance.

It is understood, that in such polyhydroxyl, polyamino, carboxylic acid, sulfuric acid, and like linkers that include free hydrogens bound to heteroatoms, one or more of those free hydrogen atoms may be protected with the appropriate hydroxyl, amino, or acid protecting group, respectively, or alternatively may be blocked as the corresponding pro-drugs, the latter of which are selected for the particular use, such as pro-drugs that release the parent drug under general or specific physiological conditions.

In each of the foregoing illustrative examples of linkers L, there are also included in some cases additional spacer linkers L_{S}, and/or additional releasable linkers L_{R}. Those spacer linker and releasable linkers also may include asymmetric carbon atoms. It is to be further understood that the stereochemical configurations shown herein are merely illustrative, and other stereochemical configurations are contemplated. For example in one variation, the corresponding unnatural amino acid configurations may be included in the conjugated described herein as follows:

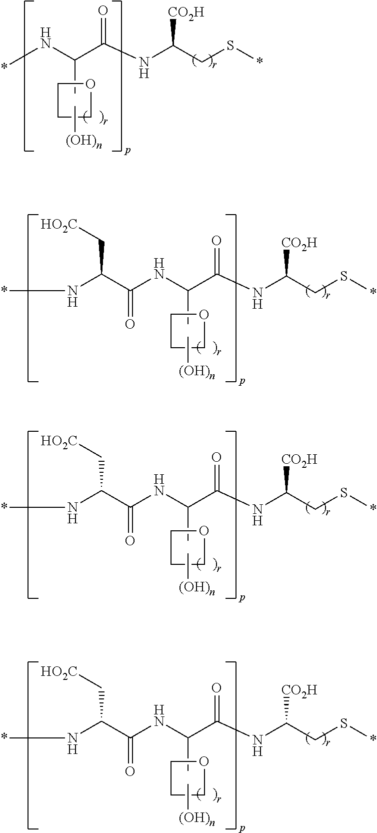

wherein n is an integer from 2 to about 5, p is an integer from 1 to about 5, and r is an integer from 1 to about 4, as described above.

Additional linkers that include hydrophilic groups useful in preparing the conjugates described herein are described in U.S. provisional application Serial Nos. 60/946,092 and 61/036,186, and in PCT international publication No. WO 2009/002993, the disclosures of which are incorporated herein by reference.

In another embodiment, multi-drug conjugates are described herein. Several illustrative configurations of such multi-drug conjugates are contemplated herein, and include those described in PCT international publication No. WO 2007/022494, the disclosure of which is incorporated herein by reference. Illustratively, the polyvalent linkers may connect the receptor binding ligand B to the two or more agents A, providing that one agent is a tubulysin. Such polyvalent conjugates may be in a variety of structural configurations, including but not limited to the following illustrative general formulae:

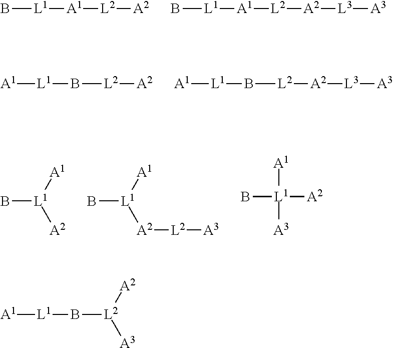

where B is the receptor binding ligand, each of (L¹), (L²), and (L³) is a polyvalent linker as described herein comprising a hydrophilic spacer linker, and optionally including one or more releasable linkers and/or additional spacer linkers, and each of (A¹), (A²), and (A³) is an agent A, or an analog or derivative thereof. Other variations, including additional agents A, or analogs or derivatives thereof, additional linkers, and additional configurations of the arrangement of each of (B), (L), and (A), are also contemplated herein.

In one variation, more than one receptor binding ligand B is included in the delivery conjugates described herein, including but not limited to the following illustrative general formulae:

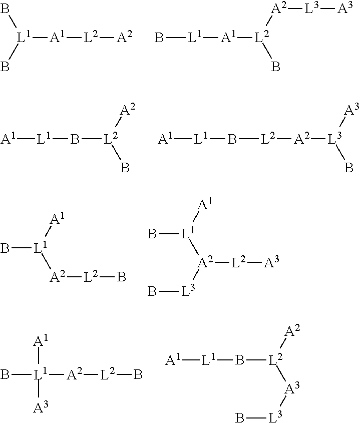

where each B is a receptor binding ligand, each of (L¹), (L²), and (L³) is a polyvalent linker as described herein comprising a hydrophilic spacer linker, and optionally including one or more releasable linkers and/or additional spacer linkers, and each of (A¹), (A²), and (A³) is an agent A, or an analog or derivative thereof where at least one of A is a tubulysin. Other variations, including additional agents A, or analogs or derivatives thereof, additional linkers, and additional configurations of the arrangement of each of (B), (L), and (A), are also contemplated herein. In one variation, the receptor binding ligands B are ligands for the same receptor, and in another variation, the receptor binding ligands B are ligands for different receptors.

The binding site for the binding ligand (B), such as a vitamin, can include receptors for any binding ligand (B), or a derivative or analog thereof, capable of specifically binding to a receptor wherein the receptor or other protein is uniquely expressed, overexpressed, or preferentially expressed by a population of pathogenic cells. A surface-presented protein uniquely expressed, overexpressed, or preferentially expressed by the pathogenic cells is typically a receptor that is either not present or present at lower concentrations on non-pathogenic cells providing a means for selective elimination of the pathogenic cells. The conjugates may be capable of high affinity binding to receptors on cancer cells or other types of pathogenic cells. The high affinity binding can be inherent to the binding ligand or the binding affinity can be enhanced by the use of a chemically modified ligand (e.g., an analog or a derivative of a vitamin).

The conjugates described herein can be formed from, for example, a wide variety of vitamins or receptor-binding vitamin analogs/derivatives, linkers, and tubulysins. The conjugates described herein are capable of selectively targeting a population of pathogenic cells in the host animal due to preferential expression of a receptor for the binding ligand, such as a vitamin, accessible for ligand binding, on the pathogenic cells. Illustrative vitamin moieties that can be used as the binding ligand (B) include carnitine, inositol, lipoic acid, pyridoxal, ascorbic acid, niacin, pantothenic acid, folic acid, riboflavin, thiamine, biotin, vitamin B₁₂, and the lipid soluble vitamins A, D, E and K. These vitamins, and their receptor-binding analogs and derivatives, constitute an illustrative targeting entity that can be coupled with the tubulysin by a bivalent linker (L) to form a binding ligand (B) conjugate as described herein. The term vitamin is understood to include vitamin analogs and/or derivatives, unless otherwise indicated. Illustratively, pteroic acid which is a derivative of folate, biotin analogs such as biocytin, biotin sulfoxide, oxybiotin and other biotin receptor-binding compounds, and the like, are considered to be vitamins, vitamin analogs, and vitamin derivatives. It should be appreciated that vitamin analogs or derivatives as described herein refer to vitamins that incorporates an heteroatom through which the vitamin analog or derivative is covalently bound to the bivalent linker (L).

Illustrative vitamin moieties include folic acid, biotin, riboflavin, thiamine, vitamin B₁₂, and receptor-binding analogs and derivatives of these vitamin molecules, and other related vitamin receptor binding molecules.

In one embodiment, the binding ligand B is a folate, an analog of folate, or a derivative of folate. It is to be understood as used herein, that the term folate is used both individually and collectively to refer to folic acid itself, and/or to such analogs and derivatives of folic acid that are capable of binding to folate receptors.

Illustrative embodiments of folate analogs and/or derivatives include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate. The dideaza analogs include, for example, 1,5-dideaza, 5,10-dideaza, 8,10-dideaza, and 5,8-dideaza analogs of folate. Other folates useful as complex forming ligands include the folate receptor-binding analogs aminopterin, amethopterin (methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). The foregoing folic acid analogs and/or derivatives are conventionally termed folates, reflecting their ability to bind with folate-receptors, and such ligands when conjugated with exogenous molecules are effective to enhance transmembrane transport, such as via folate-mediated endocytosis as described herein. Other suitable binding ligands capable of binding to folate receptors to initiate receptor mediated endocytotic transport of the complex include antibodies to the folate receptor. An exogenous molecule in complex with an antibody to a folate receptor is used to trigger transmembrane transport of the complex.

Additional analogs of folic acid that bind to folic acid receptors are described in US Patent Application Publication Serial Nos. 2005/0227985 and 2004/0242582, the disclosures of which are incorporated herein by reference. Illustratively, such folate analogs have the general formula:

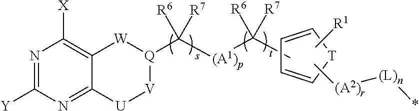

wherein X and Y are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;

L is a divalent linker as described herein; and

n, p, r, s and t are each independently either 0 or 1.

As used herein, it is to be understood that the term folate refers both individually to folic acid used in forming a conjugate, or alternatively to a folate analog or derivative thereof that is capable of binding to folate or folic acid receptors.

The folate can include a nitrogen, and in this embodiment, the spacer linkers can be alkylenecarbonyl, cycloalkylenecarbonyl, carbonylalkylcarbonyl, 1-alkylenesuccinimid-3-yl, 1-(carbonylalkyl)succinimid-3-yl, wherein each of the spacer linkers is optionally substituted with a substituent X¹, and the spacer linker is bonded to the folate nitrogen to form an imide or an alkylamide. In this embodiment, the substituents X¹ can be alkyl, hydroxyalkyl, amino, aminoalkyl, alkylaminoalkyl, dialkylaminoalkyl, sulfhydrylalkyl, alkylthioalkyl, aryl, substituted aryl, arylalkyl, substituted arylalkyl, carboxy, carboxyalkyl, guanidinoalkyl, R⁴-carbonyl, R⁵-carbonylalkyl, R⁶-acylamino, and R⁷-acylaminoalkyl, wherein R⁴ and R⁵ are each independently selected from amino acids, amino acid derivatives, and peptides, and wherein R⁶ and R⁷ are each independently selected from amino acids, amino acid derivatives, and peptides.

In another embodiment, methods for treating diseases caused by or evidenced by pathogenic cell populations are described herein. The binding ligand (B) conjugates can be used to treat disease states characterized by the presence of a pathogenic cell population in the host (e.g. a human patient) wherein the members of the pathogenic cell population have an accessible binding site for the binding ligand (B), or analog or derivative thereof, wherein the binding site is uniquely expressed, overexpressed, or preferentially expressed by the pathogenic cells. The selective elimination of the pathogenic cells is mediated by the binding of the ligand moiety of the binding ligand (B) conjugate to a ligand receptor, transporter, or other surface-presented protein that specifically binds the binding ligand (B), or analog or derivative thereof, and which is uniquely expressed, overexpressed, or preferentially expressed by the pathogenic cells. A surface-presented protein uniquely expressed, overexpressed, or preferentially expressed by the pathogenic cells is a receptor not present or present at lower concentrations on non-pathogenic cells providing a means for selective elimination of the pathogenic cells.

In one illustrative embodiment of the invention, a method is described for treating a patient with cancer, the method comprising the step of administering to the patient a composition comprising a conjugate of a tubulysin of the formula

B-L-D

or a pharmaceutically acceptable salt, isomer, mixture of isomers, crystalline form, non-crystalline form, hydrate, or solvate thereof; wherein

B is a folate;

L is a bivalent linker of the formula

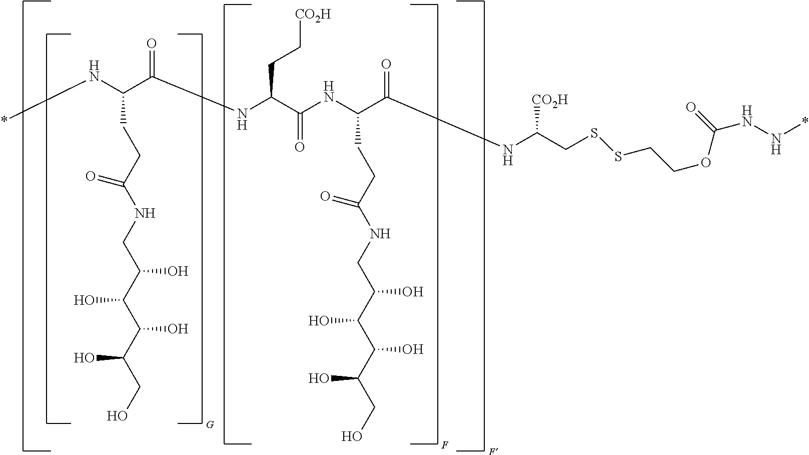

wherein *'s indicate the points of attachment, and F, F′, and G are each independently 1, 2, 3 or 4; and D is a tubulysin.

In another illustrative embodiment of the invention, a method is described for treating a patient with cancer, the method comprising the step of administering to the patient a composition comprising a conjugate of tubulysin of the formula

B-L-D

or a pharmaceutically acceptable salt, isomer, mixture of isomers, crystalline form, non-crystalline form, hydrate, or solvate thereof; wherein

B is a folate;

L is a bivalent linker of the formula

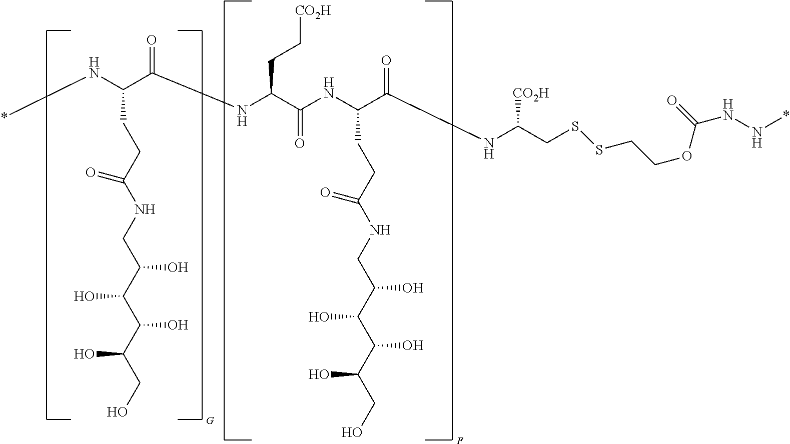

wherein *'s indicate the points of attachment, and F and G are each independently 1, 2, 3 or 4; and D is tubulysin B.

In another embodiment, the method of the preceding embodiments is described wherein the folate is of the formula

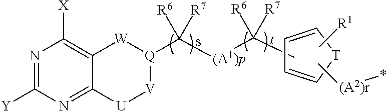

wherein * indicates the point of attachment;

X and Y are each-independently selected from the group consisting of halo, R², OR², SR³, and NR⁴R⁵;

U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;

A¹ and A² are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡H)—, C₁-C₁₂ alkylene, and C₁-C₁₂ alkyeneoxy, where Z is oxygen or sulfur;

R¹ is selected-from the group consisting of hydrogen, halo, C₁-C₁₂ alkyl, and C₁-C₁₂ alkoxy; R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;

R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group; R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group; and n, p, r, s and t are each independently either 0 or 1.

In another embodiment, the method of any one of the preceding claims is described wherein the folate has the formula

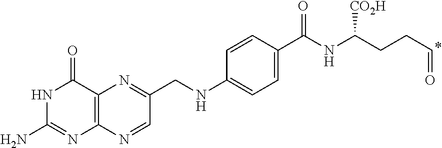

where * indicates the point of attachment;

F is 1 or 2; G is 1; F′ is 1 or 2; and

the tubulysin has the formula

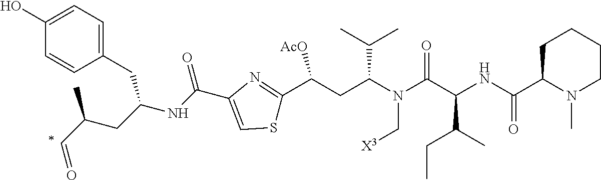

where * indicates the point of attachment, X³ is CH₃CH₂CH₂CO₂ or (CH3)₂CHCH₂CO₂.

In another embodiment, the method of any of the preceding embodiments is described wherein the folate is of the formula

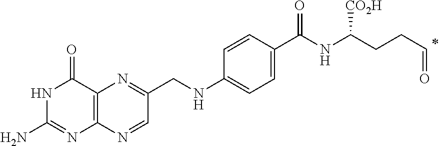

wherein * indicates the point of attachment. In still another embodiment, the method of any of the preceding embodiments wherein F is 2 and G is 1 is described.

In another embodiment, the method of any of the preceding embodiments wherein the conjugate of tubulysin is of the formula

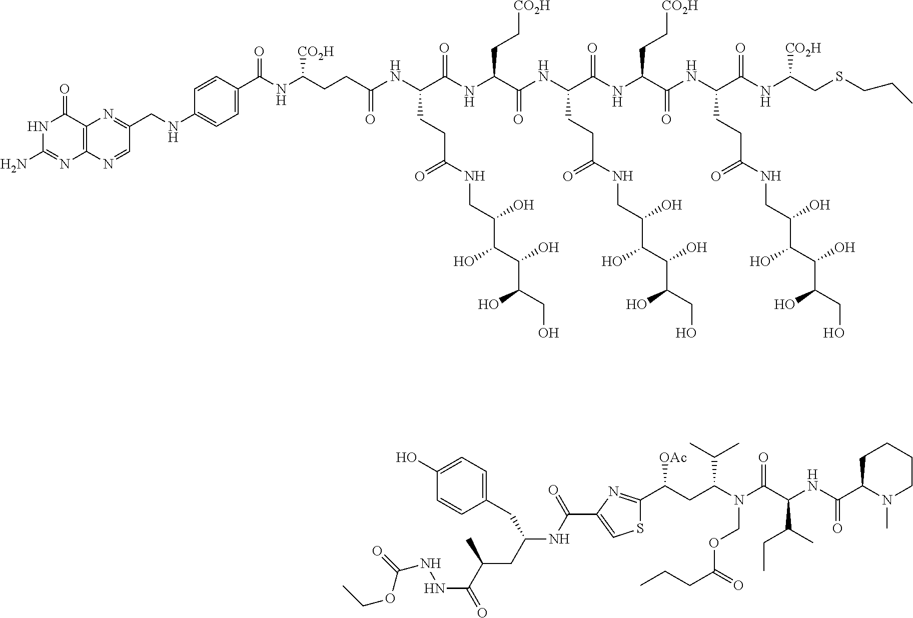

is described.

In another embodiment, the method of any one of the preceding embodiments wherein the conjugate of tubulysin is of the formula

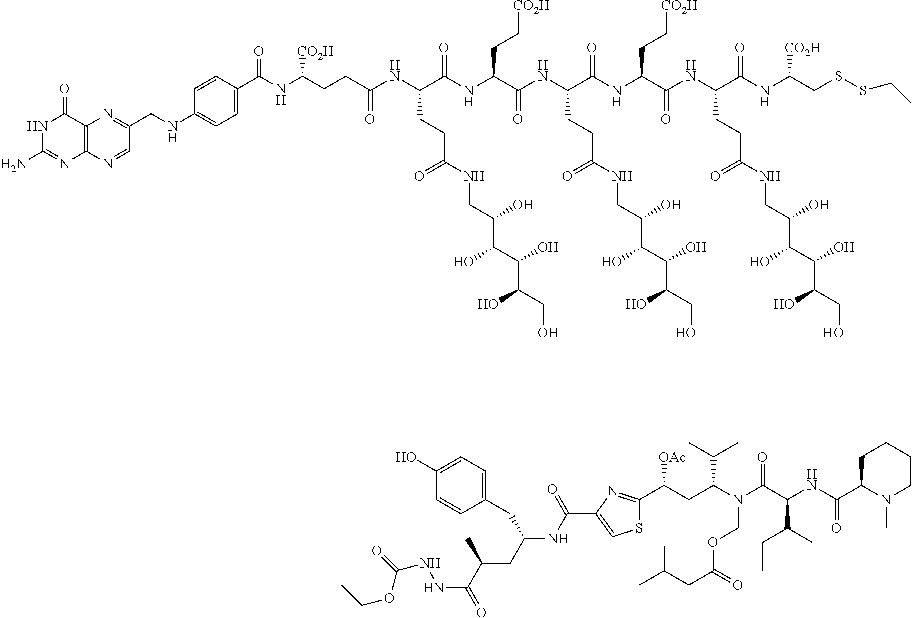

is described.

In another embodiment, the method of any one of the preceding embodiments wherein the conjugate of tubulysin is of the formula

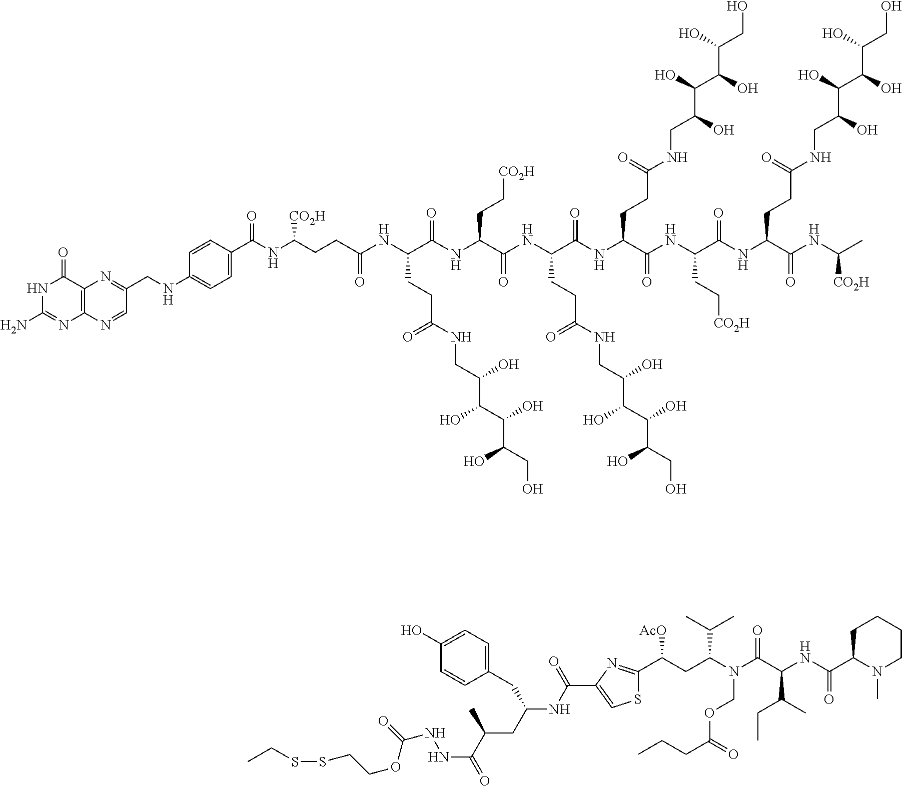 [SMILES: C[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)CC[C@H](NC(=O)c1ccc(NCc2cnc3nc(N)[nH]c(=O)c3n2)cc1)C(=O)O)C(=O)O ; CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSCC)cs1)C(C)C]

is described.

In another embodiment, the method of any one of the preceding embodiments wherein the conjugate of tubulysin is of the formula

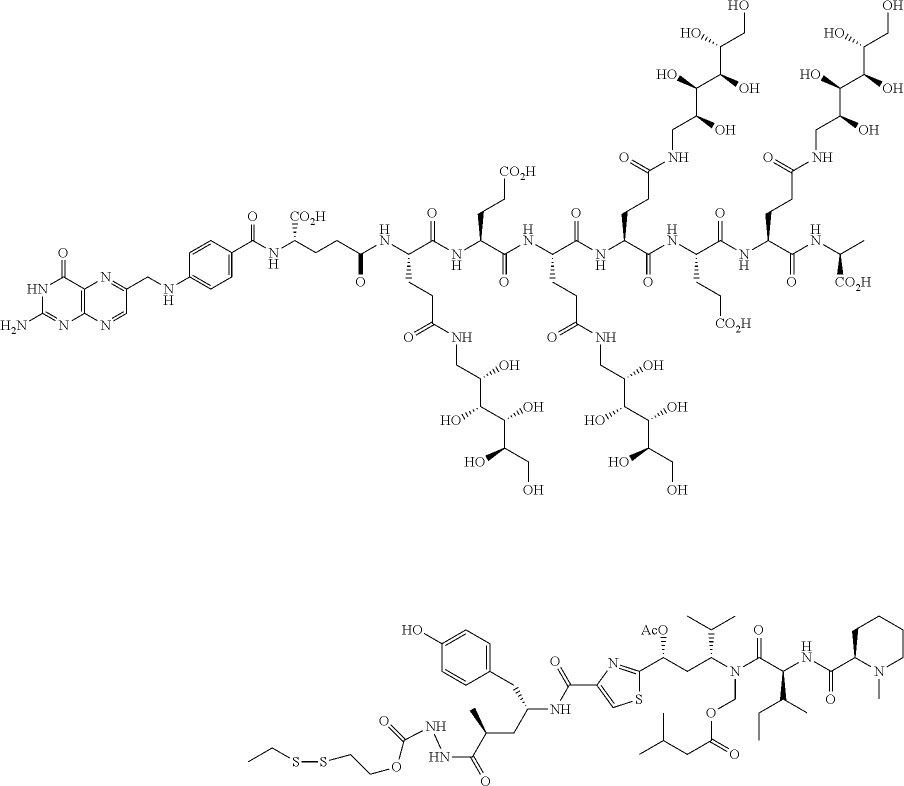 [SMILES: C[C@H](NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)NC(=O)[C@H](CCC(=O)NC[C@H](O)[C@@H](O)[C@H](O)[C@H](O)CO)NC(=O)[C@H](CCC(=O)O)NC(=O)[C@H](CCC(=O)NC[C@H](O)[C@@H](O)[C@H](O)[C@H](O)CO)N[C@@H](O)CC[C@H](NC(=O)c1ccc(NCc2cnc3nc(N)[nH]c(=O)c3n2)cc1)C(=O)O)C(=O)O ; CCSSCCOC(=O)NNC(=O)[C@@H](C)C[C@H](Cc1ccc(O)cc1)NC(=O)c1csc([C@@H](C[C@H](C(C)C)N(COC(=O)CC(C)C)C(=O)[C@@H](NC(=O)[C@H]2CCCCN2C)C(C)CC)OC(C)=O)n1]

is described.

In another embodiment, the method of any of the preceding embodiments wherein the composition further comprises one or more carriers, diluents, or excipients, or a combination thereof is described.

In another embodiment, any of the preceding embodiments wherein the purity of the conjugate is at least 95% is described. In another embodiment, any of the preceding embodiments wherein the purity of the conjugate is at least 96% is described. In another embodiment, any of the preceding embodiments wherein the purity of the conjugate is at least 96.5% is described. In another embodiment, any of the preceding embodiments wherein the purity of the conjugate is at least 97% is described. In another embodiment, any of the preceding embodiments wherein the purity of the conjugate is at least 97.5% is described. In another embodiment, any of the preceding embodiments wherein the purity of the conjugate is at least 98% is described. In another embodiment, any of the preceding embodiments wherein the purity of the conjugate is at least 98.5% is described. In another embodiment, any of the preceding embodiments wherein the purity of the conjugate is at least 99% is described. In another embodiment, any of the preceding embodiments wherein the purity of the conjugate is at least 99.5% is described.

As used herein the term “conjugate” includes conjugates of a tubulysin and tubulysin conjugates. In another embodiment, the method of any of the preceding embodiments wherein the composition is in a dosage form adapted for parenteral administration is described. In another embodiment, the method of any of the preceding embodiments wherein the dose of the conjugate of tubulysin is in the range of 1 to 5 μg/kg is described. In another embodiment, the method of any of the preceding embodiments wherein the dose of the conjugate of tubulysin is in the range of 1 to 3 μg/kg is described.

For example, surface-expressed vitamin receptors, such as the high-affinity folate receptor, are overexpressed on cancer cells. Epithelial cancers of the ovary, mammary gland, colon, lung, nose, throat, and brain have all been reported to express elevated levels of the folate receptor. In fact, greater than 90% of all human ovarian tumors are known to express large amounts of this receptor. Accordingly, the binding ligand (B)conjugates described herein can be used to treat a variety of tumor cell types, as well as other types of pathogenic cells, such as infectious agents, that preferentially express ligand receptors, such as vitamin receptors, and, thus, have surface accessible binding sites for ligands, such as vitamins (e.g. folate), or vitamin analogs or derivatives. In one aspect, methods are described herein for targeting binding ligand conjugates to maximize targeting of the pathogenic cells for elimination.

The binding ligand (B) conjugates described herein can be used for both human clinical medicine and veterinary applications. Thus, the host animal harboring the population of pathogenic cells and treated with the binding ligand (e.g., a vitamin or a folate) conjugates can be human or, in the case of veterinary applications, can be a laboratory, agricultural, domestic, or wild animal. The methods described herein can be applied to host animals including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, and wild animals in captivity such as bears, pandas, lions, tigers, leopards, elephants, zebras, giraffes, gorillas, dolphins, and whales.

Thus, the population of pathogenic cells can be a cancer cell population that is tumorigenic, including benign tumors and malignant tumors, or it can be non-tumorigenic. The cancer cell population can arise spontaneously or by such processes as mutations present in the germline of the host animal or somatic mutations, or it can be chemically-, virally-, or radiation-induced. The methods can be utilized to treat such cancers as carcinomas, sarcomas, lymphomas, Hodgkin's disease, melanomas, mesotheliomas, Burkitt's lymphoma, nasopharyngeal carcinomas, leukemias, and myelomas. The cancer cell population can include, but is not limited to, oral, thyroid, endocrine, skin, gastric, esophageal, laryngeal, pancreatic, colon, bladder, bone, ovarian, cervical, uterine, breast, testicular, prostate, rectal, kidney, liver, and lung cancers.

In embodiments where the pathogenic cell population is a cancer cell population, the effect of conjugate administration is a therapeutic response measured by reduction or elimination of tumor mass or of inhibition of tumor cell proliferation. In the case of a tumor, the elimination can be an elimination of cells of the primary tumor or of cells that have metastasized or are in the process of dissociating from the primary tumor. A prophylactic treatment with the binding ligand (B) conjugate (e.g., a vitamin or a folate used as the binding ligand) to prevent return of a tumor after its removal by any therapeutic approach including surgical removal of the tumor, radiation therapy, chemotherapy, or biological therapy is also described. The prophylactic treatment can be an initial treatment with the binding ligand (B) conjugate, such as treatment in a multiple dose daily regimen, and/or can be an additional treatment or series of treatments after an interval of days or months following the initial treatment(s). Accordingly, elimination of any of the pathogenic cell populations treated using the described methods includes reduction in the number of pathogenic cells, inhibition of proliferation of pathogenic cells, a prophylactic treatment that prevents return of pathogenic cells, or a treatment of pathogenic cells that results in reduction of the symptoms of disease.

In cases where cancer cells are being eliminated, the methods can be used in combination with surgical removal of a tumor, radiation therapy, chemotherapy, or biological therapies such as other immunotherapies including, but not limited to, monoclonal antibody therapy, treatment with immunomodulatory agents, adoptive transfer of immune effector cells, treatment with hematopoietic growth factors, cytokines and vaccination.

In one embodiment, the binding ligand conjugates can be internalized into the targeted pathogenic cells upon binding of the binding ligand moiety to a receptor, transporter, or other surface-presented protein that specifically binds the ligand and which is preferentially expressed on the pathogenic cells. Such internalization can occur, for example, through receptor-mediated endocytosis. If the binding ligand (B) conjugate contains a releasable linker, the binding ligand moiety and the tubulysin can dissociate intracellularly and the tubulysin can act on its intracellular target.

In an alternate embodiment, the binding ligand moiety of the conjugate can bind to the pathogenic cell placing the tubulysin in close association with the surface of the pathogenic cell. The tubulysin can then be released by cleavage of the releasable linker. For example, the tubulysin can be released by a protein disulfide isomerase if the releasable linker is a disulfide group. The tubulysin can then be taken up by the pathogenic cell to which the binding ligand (B) conjugate is bound, or the drug can be taken up by another pathogenic cell in close proximity thereto. Alternatively, the drug could be released by a protein disulfide isomerase inside the cell where the releasable linker is a disulfide group. The tubulysin may also be released by a hydrolytic mechanism, such as acid-catalyzed hydrolysis, as described above for certain beta elimination mechanisms, or by an anchimerically assisted cleavage through an oxonium ion or lactonium ion producing mechanism. The selection of the releasable linker or linkers will dictate the mechanism by which the tubulysin is released from the conjugate. It is appreciated that such a selection can be pre-defined by the conditions wherein the tubulysin conjugate will be used. Alternatively, the conjugates can be internalized into the targeted cells upon binding, and the binding ligand and the tubulysin can remain associated intracellularly with the tubulysin exhibiting its effects without dissociation from the folate moiety.

In still another embodiment where the binding ligand is a vitamin, the vitamin-conjugate can act through a mechanism independent of cellular vitamin receptors. For example, the conjugates can bind to soluble vitamin receptors present in the serum or to serum proteins, such as albumin, resulting in prolonged circulation of the conjugates relative to the unconjugated tubulysin, and in increased activity of the conjugates towards the pathogenic cell population relative to the unconjugated tubulysin.

In one embodiment, the tubulysin remains stable in serum for at least 4 hours. In another embodiment the tubulysin has an IC₅₀ in the nanomolar range, and, in another embodiment, the tubulysin is water soluble. If the tubulysin is not water soluble, the bivalent linker (L) can be derivatized to enhance water solubility. The term tubulysin also means any of the tubulysin analogs or derivatives described hereinabove. It should be appreciated that a tubulysin analog or derivative can mean a tubulysin that incorporates an heteroatom through which the drug analog or derivative is covalently bound to the bivalent linker (L).

The binding ligand conjugates can comprise a binding ligand (B), a bivalent linker (L), a tubulysin, and, optionally, heteroatom linkers to link the binding ligand (B) receptor binding moiety and the tubulysin to the bivalent linker (L). In one illustrative embodiment, it should be appreciated that a folate analog or derivative can mean a folate that incorporates a heteroatom through which the folate analog or derivative is covalently bound to the bivalent linker (L). Thus, in this illustrative embodiment, the folate can be covalently bound to the bivalent linker (L) through an heteroatom linker, or a folate analog or derivative (i.e., incorporating an heteroatom) can be directly bound to the bivalent linker (L). In similar illustrative embodiments, a tubulysin analog or derivative is a tubulysin, and a tubulysin analog or derivative can mean a tubulysin that incorporates a heteroatom through which the tubulysin analog or derivative is covalently bound to the bivalent linker (L). Thus, in these illustrative aspects, the tubulysin can be covalently bound to the bivalent linker (L) through an heteroatom linker, or a drug analog or derivative (i.e., incorporating an heteroatom) can be directly bound to the bivalent linker (L). The bivalent linker (L) can comprise a spacer linker, a releasable (i.e., cleavable) linker, and an heteroatom linker to link the spacer linker to the releasable linker in conjugates containing both of these types of linkers.

Generally, any manner of forming a conjugate between the bivalent linker (L) and the binding ligand (B), or analog or derivative thereof, between the bivalent linker (L) and the tubulysin, or analog or derivative thereof, including any intervening heteroatom linkers, can be utilized. Also, any art-recognized method of forming a conjugate between the spacer linker, the releasable linker, and the heteroatom linker to form the bivalent linker (L) can be used. The conjugate can be formed by direct conjugation of any of these molecules, for example, through complexation, or through hydrogen, ionic, or covalent bonds. Covalent bonding can occur, for example, through the formation of amide, ester, disulfide, or imino bonds between acid, aldehyde, hydroxy, amino, sulfhydryl, or hydrazo groups.

In another embodiment, pharmaceutical compositions comprising an amount of a binding ligand (B) conjugate effective to eliminate a population of pathogenic cells in a host animal (e.g. a human patient) when administered in one or more doses are described. The binding ligand drug delivery conjugate is preferably administered to the host animal parenterally, e.g., intradermally, subcutaneously, intramuscularly, intraperitoneally, intravenously, or intrathecally. Alternatively, the binding ligand drug delivery conjugate can be administered to the host animal by other medically useful processes, such as orally, and any effective dose and suitable therapeutic dosage form, including prolonged release dosage forms, can be used.

In other embodiments of the methods described herein, pharmaceutically acceptable salts of the conjugates described herein are described. Pharmaceutically acceptable salts of the conjugates described herein include the acid addition and base salts thereof.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the conjugates described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemi-salts of acids and bases may also be formed, for example, hemi-sulphate and hemi-calcium salts.

In various embodiments of the methods described herein, the conjugates described herein may be administered alone or in combination with one or more other conjugates described herein or in combination with one or more other drugs (or as any combination thereof). In one embodiment, the conjugates described herein may be administered as a formulation in association with one or more pharmaceutically acceptable carriers. The carriers can be excipients. The term “carrier” is used herein to describe any ingredient other than a conjugate described herein. The choice of carrier will to a large extent depend on factors such as the particular mode of administration, the effect of the carrier on solubility and stability, and the nature of the dosage form. Pharmaceutical compositions suitable for the delivery of conjugates described herein and methods for their preparation will be readily apparent to those skilled in the art. Such compositions and methods for their preparation may be found, for example, in Remington: The Science & Practice of Pharmacy, 21th Edition (Lippincott Williams & Wilkins, 2005), incorporated herein by reference.

In one illustrative aspect, a pharmaceutically acceptable carrier includes any and all solvents, dispersion media, coatings, antibacterial and antifungal agents, isotonic and absorption delaying agents, and the like, and combinations thereof, that are physiologically compatible. In some embodiments, the carrier is suitable for parenteral administration. Pharmaceutically acceptable carriers include sterile aqueous solutions or dispersions and sterile powders for the extemporaneous preparation of sterile injectable solutions or dispersions. Supplementary active compounds can also be incorporated into compositions of the invention.

In various embodiments, liquid formulations may include suspensions and solutions. Such formulations may comprise a carrier, for example, water, ethanol, polyethylene glycol, propylene glycol, methylcellulose or a suitable oil, and one or more emulsifying agents and/or suspending agents. Liquid formulations may also be prepared by the reconstitution of a solid, for example, from a sachet.

In one embodiment, an aqueous suspension may contain the conjugates described herein in admixture with appropriate excipients. Such excipients are suspending agents, for example, sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide, for example, lecithin; a condensation product of an alkylene oxide with a fatty acid, for example, polyoxyethylene stearate; a condensation product of ethylene oxide with a long chain aliphatic alcohol, for example, heptadecaethyleneoxycetanol; a condensation product of ethylene oxide with a partial ester derived from fatty acids and a hexitol such as polyoxyethylene sorbitol monooleate; or a condensation product of ethylene oxide with a partial ester derived from fatty acids and hexitol anhydrides, for example, polyoxyethylene sorbitan monooleate. The aqueous suspensions may also contain one or more preservatives, for example, ascorbic acid, ethyl, n-propyl, or p-hydroxybenzoate; or one or more coloring agents.

In one illustrative embodiment, dispersible powders and granules suitable for preparation of an aqueous suspension by the addition of water provide the conjugate in admixture with a dispersing or wetting agent, suspending agent and one or more preservatives. Additional excipients, for example, coloring agents, may also be present.

Suitable emulsifying agents may be naturally-occurring gums, for example, gum acacia or gum tragacanth; naturally-occurring phosphatides, for example, soybean lecithin; and esters including partial esters derived from fatty acids and hexitol anhydrides, for example, sorbitan mono-oleate, and condensation products of the said partial esters with ethylene oxide, for example, polyoxyethylene sorbitan monooleate.

In other embodiments, isotonic agents, for example, sugars, polyalcohols such as mannitol, sorbitol, or sodium chloride can be included in the composition. Prolonged absorption of the injectable compositions can be brought about by including in the composition an agent which delays absorption, for example, monostearate salts and gelatin.

In one aspect, a conjugate as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors and infusion techniques.

Examples of parenteral dosage forms include aqueous solutions of the active agent, in an isotonic saline, 5% glucose or other well-known pharmaceutically acceptable liquid carriers such as liquid alcohols, glycols, esters, and amides. The parenteral dosage form can be in the form of a reconstitutable lyophilizate comprising the dose of the conjugate. In one aspect of the present embodiment, any of a number of prolonged release dosage forms known in the art can be administered such as, for example, the biodegradable carbohydrate matrices described in U.S. Pat. Nos. 4,713,249; 5,266,333; and 5,417,982, the disclosures of which are incorporated herein by reference, or, alternatively, a slow pump (e.g., an osmotic pump) can be used.

In one illustrative aspect, parenteral formulations are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water. In other embodiments, any of the liquid formulations described herein may be adapted for parenteral administration of the conjugates described herein. The preparation of parenteral formulations under sterile conditions, for example, by lyophilization under sterile conditions, may readily be accomplished using standard pharmaceutical techniques well-known to those skilled in the art. In one embodiment, the solubility of a conjugate used in the preparation of a parenteral formulation may be increased by the use of appropriate formulation techniques, such as the incorporation of solubility-enhancing agents.

In various embodiments, formulations for parenteral administration may be formulated to be for immediate and/or modified release. In one illustrative aspect, active agents of the invention may be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PGLA). Methods for the preparation of such formulations are generally known to those skilled in the art. In another embodiment, the conjugates described herein or compositions comprising the conjugates may be continuously administered, where appropriate.

In one embodiment, sterile injectable solutions can be prepared by incorporating the conjugate in the required amount in an appropriate solvent with one or a combination of ingredients described above, as required, followed by filtered sterilization. Typically, dispersions are prepared by incorporating the conjugate into a sterile vehicle which contains a dispersion medium and any additional ingredients from those described above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. In one embodiment, the proper fluidity can be maintained, for example, by the use of a coating such as lecithin, by the maintenance of the required particle size in the case of dispersion and by the use of surfactants.

In one illustrative aspect, at least one additional composition comprising a therapeutic factor can be administered to the host in combination or as an adjuvant to the above-detailed methodology, to enhance the binding ligand conjugate-mediated elimination of the population of pathogenic cells, or more than one additional therapeutic factor can be administered. The therapeutic factor can be selected from a chemotherapeutic agent, or another therapeutic factor capable of complementing the efficacy of the administered binding ligand drug delivery conjugate.

In one illustrative aspect, therapeutically effective combinations of these factors can be used. In one embodiment, for example, therapeutically effective amounts of the therapeutic factor, for example, in amounts ranging from about 0.1 MIU/m²/dose/day to about 15 MIU/m²/dose/day in a multiple dose daily regimen, or for example, in amounts ranging from about 0.1 MIU/m²/dose/day to about 7.5 MIU/m²/dose/day in a multiple dose daily regimen, can be used along with the binding ligand conjugates to eliminate, reduce, or neutralize pathogenic cells in a host animal harboring the pathogenic cells (MIU=million international units; m²=approximate body surface area of an average human).

In another embodiment, chemotherapeutic agents, which are, for example, cytotoxic themselves or can work to enhance tumor permeability, are also suitable for use in the described methods in combination with the binding ligand conjugates. Such chemotherapeutic agents include adrenocorticoids and corticosteroids, alkylating agents, antiandrogens, antiestrogens, androgens, aclamycin and aclamycin derivatives, estrogens, antimetabolites such as cytosine arabinoside, purine analogs, pyrimidine analogs, and methotrexate, busulfan, carboplatin, chlorambucil, cisplatin and other platinum compounds, tamoxiphen, taxol, paclitaxel, paclitaxel derivatives, Taxotere®, cyclophosphamide, daunomycin, rhizoxin, T2 toxin, plant alkaloids, prednisone, hydroxyurea, teniposide, mitomycins, discodermolides, microtubule inhibitors, epothilones, tubulysin, cyclopropyl benz[e]indolone, seco-cyclopropyl benz[e]indolone, O-Ac-seco-cyclopropyl benz[e]indolone, bleomycin and any other antibiotic, nitrogen mustards, nitrosureas, vincristine, vinblastine, and analogs and derivative thereof such as deacetylvinblastine monohydrazide, colchicine, colchicine derivatives, allocolchicine, thiocolchicine, trityl cysteine, Halicondrin B, dolastatins such as dolastatin 10, amanitins such as α-amanitin, camptothecin, irinotecan, and other camptothecin derivatives thereof, geldanamycin and geldanamycin derivatives, estramustine, nocodazole, MAP4, colcemid, inflammatory and proinflammatory agents, peptide and peptidomimetic signal transduction inhibitors, and any other art-recognized drug or toxin. Other drugs that can be used include penicillins, cephalosporins, vancomycin, erythromycin, clindamycin, rifampin, chloramphenicol, aminoglycoside antibiotics, gentamicin, amphotericin B, acyclovir, trifluridine, ganciclovir, zidovudine, amantadine, ribavirin, maytansines and analogs and derivatives thereof, gemcitabine, and any other art-recognized antimicrobial compound.

The therapeutic factor can be administered to the host animal prior to, after, or at the same time as the binding ligand conjugates and the therapeutic factor can be administered as part of the same composition containing the binding delivery conjugate or as part of a different composition than the binding ligand conjugate. Any such therapeutic composition containing the therapeutic factor at a therapeutically effective dose can be used.

Also contemplated herein are kits comprising the conjugates described herein. In another embodiment, a kit comprising a sterile vial, the composition of any one of the preceding claims, and instructions for use describing use of the composition for treating a patient with cancer is described.

In another embodiment, the kit of the preceding embodiment wherein the composition is in the form of a reconstitutable lyophlizate is described.

In another embodiment, the method of any of the preceding kit embodiments wherein the dose of the conjugate of tubulysin is in the range of 1 to 5 μg/kg is described.

In another embodiment, the method of any of the preceding kit embodiments wherein the dose of the conjugate of tubulysin is in the range of 1 to 3 μg/kg is described.

In another embodiment, the method of any of the preceding kit embodiments wherein the purity of the conjugate of tubulysin is at least 98% is described.

Additionally, more than one type of binding delivery conjugate can be used. Illustratively, for example, the host animal can be treated with conjugates with different vitamins, conjugate to a tubulysin in a co-dosing protocol. In other embodiments, the host animal can be treated with conjugates comprising the same binding ligand linked to different drugs, or various binding ligands linked to various drugs. In another illustrative embodiment, binding ligand conjugates with the same or different vitamins, and the same or different drugs comprising multiple vitamins and multiple drugs as part of the same drug delivery conjugate could be used.

The unitary daily dosage of the binding delivery conjugate can vary significantly depending on the host condition, the disease state being treated, the molecular weight of the conjugate, its route of administration and tissue distribution, and the possibility of co-usage of other therapeutic treatments such as radiation therapy. The effective amount to be administered to a patient is based on body surface area, patient weight, and physician assessment of patient condition. In illustrative embodiments, effective doses can range, for example, from about 1 ng/kg to about 1 mg/kg, from about 1 μg/kg to about 500 μg/kg, from about 1 μg/kg to about 100 μg/kg, and from about 0.01 ng/kg to about 5 μg/kg.

In another illustrative aspect, any effective regimen for administering the binding ligand conjugates can be used. For example, the binding ligand conjugates can be administered as single doses, or can be divided and administered as a multiple-dose daily regimen. In other embodiments, a staggered regimen, for example, one to three days per week can be used as an alternative to daily treatment, and such intermittent or staggered daily regimen is considered to be equivalent to every day treatment and within the scope of the methods described herein. In one embodiment, the host is treated with multiple injections of the binding ligand conjugate to eliminate the population of pathogenic cells. In another embodiment, the host is injected multiple times (preferably about 2 up to about 50 times) with the binding ligand conjugate, for example, at 12-72 hour intervals or at 48-72 hour intervals. In other embodiments, additional injections of the binding ligand conjugate can be administered to the patient at an interval of days or months after the initial injections(s) and the additional injections prevent recurrence of the disease state caused by the pathogenic cells.

The conjugates described herein can be prepared by art-recognized synthetic methods. The synthetic methods are chosen depending upon the selection of the optionally addition heteroatoms or the heteroatoms that are already present on the spacer linkers, releasable linkers. the drug, and/or or the binding ligand. In general, the relevant bond forming reactions are described in Richard C. Larock, “Comprehensive Organic Transformations, a guide to functional group preparations,” VCH Publishers, Inc. New York (1989), and in Theodora E. Greene & Peter G. M. Wuts, “Protective Groups ion Organic Synthesis,” 2d edition, John Wiley & Sons, Inc. New York (1991), the disclosures of which are incorporated herein by reference.

### EXAMPLES

### Compound Examples

The compounds described herein may be prepared using the process and syntheses described herein, as well as using general organic synthetic methods. In particular, methods for preparing the compounds are described in U.S. patent application publication 2005/0002942, the disclosure of which is incorporated herein by reference.

General formation of folate-peptides. The folate-containing peptidyl fragment Pte-Glu-(AA)ₘ-NH(CHR₂)CO₂H (3) is prepared by a polymer-supported sequential approach using standard methods, such as the Fmoc-strategy on an acid-sensitive Fmoc-AA-Wang resin (1), as shown in the following Scheme:

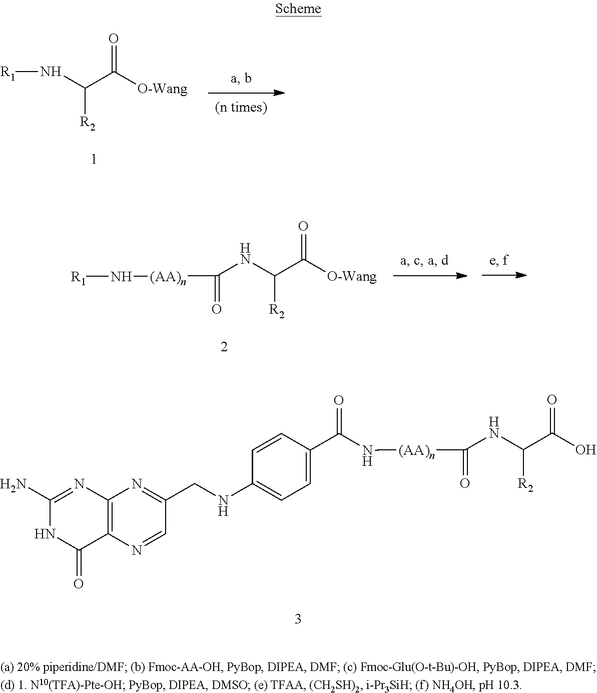

In this illustrative embodiment of the processes described herein, R₁ is Fmoc, R₂ is the desired appropriately-protected amino acid side chain, and DIPEA is diisopropylethylamine. Standard coupling procedures, such as PyBOP and others described herein or known in the art are used, where the coupling agent is illustratively applied as the activating reagent to ensure efficient coupling. Fmoc protecting groups are removed after each coupling step under standard conditions, such as upon treatment with piperidine, tetrabutylammonium fluoride (TBAF), and the like. Appropriately protected amino acid building blocks, such as Fmoc-Glu-OtBu, N¹⁰-TFA-Pte-OH, and the like, are used, as described in the Scheme, and represented in step (b) by Fmoc-AA-OH. Thus, AA refers to any amino acid starting material, that is appropriately protected. It is to be understood that the term amino acid as used herein is intended to refer to any reagent having both an amine and a carboxylic acid functional group separated by one or more carbons, and includes the naturally occurring alpha and beta amino acids, as well as amino acid derivatives and analogs of these amino acids. In particular, amino acids having side chains that are protected, such as protected serine, threonine, cysteine, aspartate, and the like may also be used in the folate-peptide synthesis described herein. Further, gamma, delta, or longer homologous amino acids may also be included as starting materials in the folate-peptide synthesis described herein. Further, amino acid analogs having homologous side chains, or alternate branching structures, such as norleucine, isovaline, β-methyl threonine, β-methyl cysteine, β,β-dimethyl cysteine, and the like, may also be included as starting materials in the folate-peptide synthesis described herein.

The coupling sequence (steps (a) & (b)) involving Fmoc-AA-OH is performed “n” times to prepare solid-support peptide (2), where n is an integer and may equal 0 to about 100. Following the last coupling step, the remaining Fmoc group is removed (step (a)), and the peptide is sequentially coupled to a glutamate derivative (step (c)), deprotected, and coupled to TFA-protected pteroic acid (step (d)). Subsequently, the peptide is cleaved from the polymeric support upon treatment with trifluoroacetic acid, ethanedithiol, and triisopropylsilane (step (e)). These reaction conditions result in the simultaneous removal of the t-Bu, t-Boc, and Trt protecting groups that may form part of the appropriately-protected amino acid side chain. The TFA protecting group is removed upon treatment with base (step (f)) to provide the folate-containing peptidyl fragment (3).

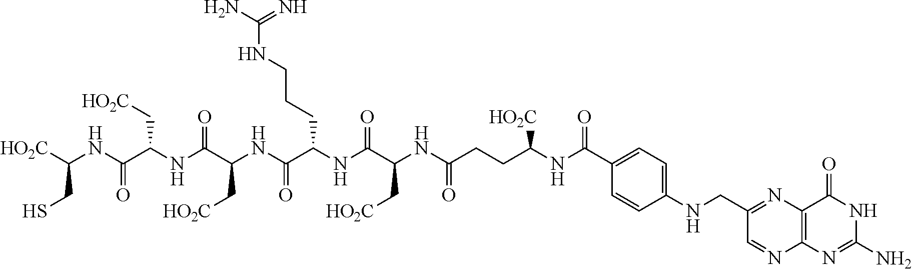 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

According to the general procedure described herein, Wang resin bound 4-methoxytrityl (MTT)-protected Cys-NH₂ was reacted according to the following sequence: 1) a. Fmoc-Asp(OtBu)-OH, PyBOP, DIPEA; b. 20% Piperidine/DMF; 2) a. Fmoc-Asp(OtBu)-OH, PyBOP, DIPEA; b. 20% Piperidine/DMF; 3) a. Fmoc-Arg(Pbf)-OH, PyBOP, DIPEA; b. 20% Piperidine/DMF; 4) a. Fmoc-Asp(OtBu)-OH, PyBOP, DIPEA; b. 20% Piperidine/DMF; 5) a. Fmoc-Glu-OtBu, PyBOP, DIPEA; b. 20% Piperidine/DMF; 6) N¹⁰-TFA-pteroic acid, PyBOP, DIPEA. The MTT, tBu, and Pbf protecting groups were removed with TFA/H₂O/TIPS/EDT (92.5:2.5:2.5:2.5), and the TFA protecting group was removed with aqueous NH₄OH at pH=9.3. Selected ¹H NMR (D₂O) δ (ppm) 8.68 (s, 1H, FA H-7), 7.57 (d, 2H, J=8.4 Hz, FA H-12 &16), 6.67 (d, 2H, J=9 Hz, FA H-13 &15), 4.40-4.75 (m, 5H), 4.35 (m, 2H), 4.16 (m, 1H), 3.02 (m, 2H), 2.55-2.95 (m, 8H), 2.42 (m, 2H), 2.00-2.30 (m, 2H), 1.55-1.90 (m, 2H), 1.48 (m, 2H); MS (ESI, m+H⁺) 1046.

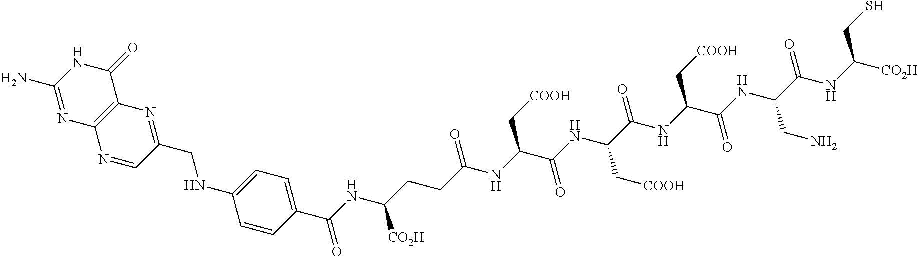 [SMILES: NC[C@H](NC(=O)[C@H](CC(=O)O)NC(=O)[C@H](CC(=O)O)NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CS)C(=O)O]

According to the general procedure described herein, Wang resin bound 4-methoxytrityl (MTT)-protected Cys-NH₂ was reacted according to the following sequence: 1) a. Fmoc-β-aminoalanine(NH-MTT)-OH, PyBOP, DIPEA; b. 20% Piperidine/DMF; 2) a. Fmoc-Asp(OtBu)-OH, PyBOP, DIPEA; b. 20% Piperidine/DMF; 3) a. Fmoc-Asp(OtBu)-OH, PyBOP, DIPEA; b. 20% Piperidine/DMF; 4) a. Fmoc-Asp(OtBu)-OH, PyBOP, DIPEA; b. 20% Piperidine/DMF; 5) a. Fmoc-Glu-OtBu, PyBOP, DIPEA; b. 20% Piperidine/DMF; 6) N¹⁰-TFA-pteroic acid, PyBOP, DIPEA. The MTT, tBu, and TFA protecting groups were removed with a. 2% hydrazine/DMF; b. TFA/H₂O/TIPS/EDT (92.5:2.5:2.5:2.5).

The reagents shown in the following table were used in the preparation:

| Reagent | (mmol) | equivalents | Amount | |
|---|---|---|---|---|
| H-Cys(4-methoxytrityl)-2- | 0.56 | 1 | 1.0 | g |
| chlorotrityl-Resin (loading | | | | |
| 0.56 mmol/g) | | | | |
| Fmoc-β-aminoalanine(NH-MTT)- | 1.12 | 2 | 0.653 | g |
| OH | | | | |
| Fmoc-Asp(OtBu)-OH | 1.12 | 2 | 0.461 | g |
| Fmoc-Asp(OtBu)-OH | 1.12 | 2 | 0.461 | g |
| Fmoc-Asp(OtBu)-OH | 1.12 | 2 | 0.461 | g |
| Fmoc-Glu-OtBu | 1.12 | 2 | 0.477 | g |
| N¹⁰TFA-Pteroic Acid | 0.70 | 1.25 | 0.286 | g |
| (dissolve in 10 ml DMSO) | | | | |
| DIPEA | 2.24 | 4 | 0.390 | mL |
| PyBOP | 1.12 | 2 | 0.583 | g |

The coupling step was performed as follows: In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3× (10 min), before each amino acid coupling. Continue to complete all 6 coupling steps. At the end wash the resin with 2% hydrazine in DMF 3× (5 min) to cleave TFA protecting group on Pteroic acid.

Cleave the peptide analog from the resin using the following reagent, 92.5% (50 ml) TFA, 2.5% (1.34 ml) H₂O, 2.5% (1.34 ml) Triisopropylsilane, 2.5% (1.34 ml) ethanedithiol, the cleavage step was performed as follows: Add 25 ml cleavage reagent and bubble for 1.5 hr, drain, and wash 3× with remaining reagent. Evaporate to about 5 mL and precipitate in ethyl ether. Centrifuge and dry. Purification was performed as follows: Column-Waters NovaPak C₁₈ 300×19 mm; Buffer A=10 mM Ammonium Acetate, pH 5; B=CAN; 1% B to 20% B in 40 minutes at 15 mL/min, to 350 mg (64%); HPLC-RT 10.307 min., 100% pure, ¹H HMR spectrum consistent with the assigned structure, and MS (ES−): 1624.8, 1463.2, 1462.3, 977.1, 976.2, 975.1, 974.1, 486.8, 477.8.

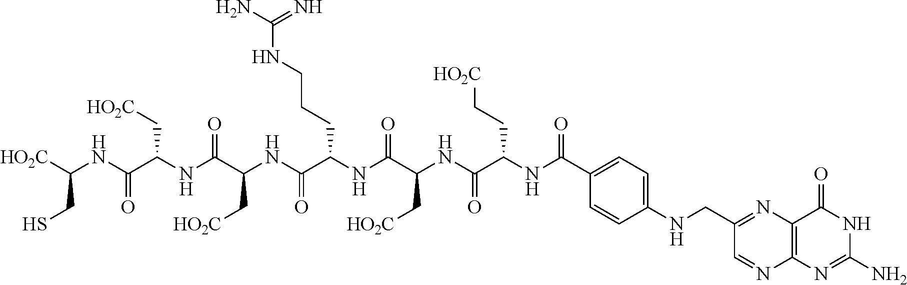 [SMILES: N=C(N)NCCC[C@H](NC(=O)[C@H](CC(=O)O)NC(=O)[C@H](CCC(=O)O)NC(=O)c1ccc(NCc2cnc3nc(N)[nH]c(=O)c3n2)cc1)C(=O)N[C@@H](CC(=O)O)C(=O)N[C@@H](CC(=O)O)C(=O)N[C@@H](CS)C(=O)O]

According to the general procedure described herein, Wang resin bound MTT-protected Cys-NH₂ was reacted according to the following sequence: 1) a. Fmoc-Asp(OtBu)-OH, PyBOP, DIPEA; b. 20% Piperidine/DMF; 2) a. Fmoc-Asp(OtBu)-OH, PyBOP, DIPEA; b. 20% Piperidine/DMF; 3) a. Fmoc-Arg(Pbf)-OH, PyBOP, DIPEA; b. 20% Piperidine/DMF; 4) a. Fmoc-Asp(OtBu)-OH, PyBOP, DIPEA; b. 20% Piperidine/DMF; 5) a. Fmoc-Glu(γ-OtBu)-OH, PyBOP, DIPEA; b. 20% Piperidine/DMF; 6) N¹⁰-TFA-pteroic acid, PyBOP, DIPEA. The MTT, tBu, and Pbf protecting groups were removed with TFA/H₂O/TIPS/EDT (92.5:2.5:2.5:2.5), and the TFA protecting group was removed with aqueous NH₄OH at pH=9.3. The ¹H NMR spectrum was consistent with the assigned structure.

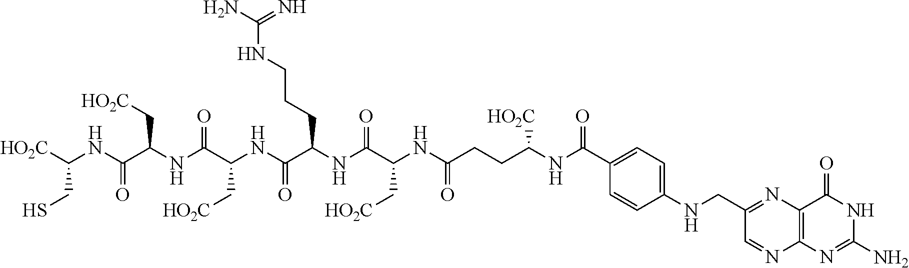 [SMILES: N=C(N)NCCC[C@@H](NC(=O)[C@@H](CC(=O)O)NC(=O)CC[C@@H](NC(=O)c1ccc(NCc2cnc3nc(N)[nH]c(=O)c3n2)cc1)C(=O)O)C(=O)N[C@H](CC(=O)O)C(=O)N[C@H](CC(=O)O)C(=O)N[C@H](CS)C(=O)O]

According to the general procedure described herein, Wang resin bound MTT-protected D-Cys-NH₂ was reacted according to the following sequence: 1) a. Fmoc-D-Asp(OtBu)-OH, PyBOP, DIPEA; b. 20% Piperidine/DMF; 2) a. Fmoc-D-Asp(OtBu)-OH, PyBOP, DIPEA; b. 20% Piperidine/DMF; 3) a. Fmoc-D-Arg(Pbf)-OH, PyBOP, DIPEA; b. 20% Piperidine/DMF; 4) a. Fmoc-D-Asp(OtBu)-OH, PyBOP, DIPEA; b. 20% Piperidine/DMF; 5) a. Fmoc-D-Glu-OtBu, PyBOP, DIPEA; b. 20% Piperidine/DMF; 6) N¹⁰-TFA-pteroic acid, PyBOP, DIPEA. The MTT, tBu, and Pbf protecting groups were removed with TFA/H₂O/TIPS/EDT (92.5:2.5:2.5:2.5), and the TFA protecting group was removed with aqueous NH₄OH at pH=9.3. The ¹H NMR spectrum was consistent with the assigned structure.

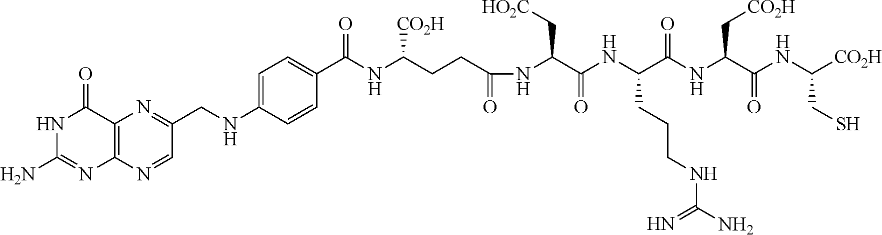 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CS)C(=O)O]

Similarly, EC089 was prepared as described herein.

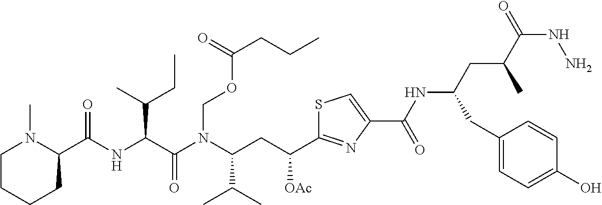 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NN)cs1)C(C)C]

Preparation of tubulysin hydrazides. Illustrated by preparing EC0347. N,N-Diisopropylethylamine (DIPEA, 6.1 μL) and isobutyl chloroformate (3.0 μL) were added with via syringe in tandem into a solution of tubulysin B (0.15 mg) in anhydrous EtOAc (2.0 mL) at −15° C. After stirring for 45 minutes at −15° C. under argon, the reaction mixture was cooled down to −20° C. and to which was added anhydrous hydrazine (5.0 μL). The reaction mixture was stirred under argon at −20° C. for 3 hours, quenched with 1.0 mM sodium phosphate buffer (pH 7.0, 1.0 mL), and injected into a preparative HPLC for purification. Column: Waters XTerra Prep MS C₁₈ 10 μm, 19×250 mm; Mobile phase A: 1.0 mM sodium phosphate buffer, pH 7.0; Mobile phase B: acetonitrile; Method: 10% B to 80% B over 20 minutes, flow rate=25 mL/min. Fractions from 15.14-15.54 minutes were collected and lyophilized to produce EC0347 as a white solid (2.7 mg). The foregoing method is equally applicable for preparing other tubulysin hydrazides by the appropriate selection of the tubulysin starting compound.

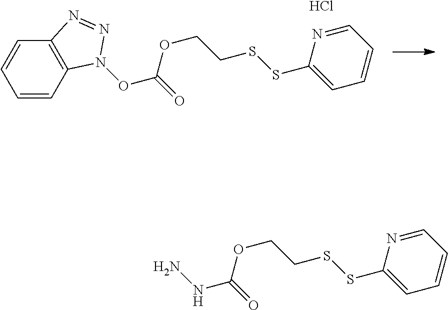 [SMILES: Cl ; O=C(OCCSSc1ccccn1)On1nnc2ccccc21 ; NNC(=O)OCCSSc1ccccn1]

Synthesis of coupling reagent EC0311. DIPEA (0.60 mL) was added to a suspension of HOBt-OCO₂—(CH₂)₂—SS-2-pyridine HCl (685 mg, 91%) in anhydrous DCM (5.0 mL) at 0° C., stirred under argon for 2 minutes, and to which was added anhydrous hydrazine (0.10 mL). The reaction mixture was stirred under argon at 0° C. for 10 minutes and room temperature for an additional 30 minutes, filtered, and the filtrate was purified by flash chromatography (silica gel, 2% MeOH in DCM) to afford EC0311 as a clear thick oil (371 mg), solidified upon standing.

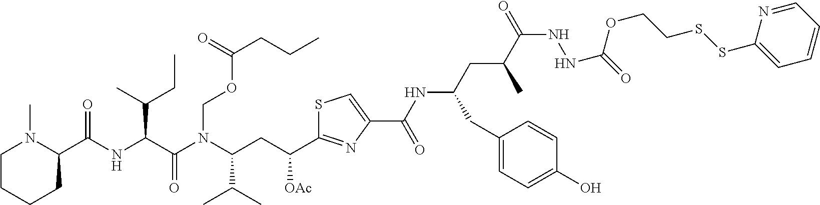 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSc2ccccn2)cs1)C(C)C]

Preparation of tubulysin disulfides (stepwise process). Illustrated for EC0312. DIPEA (36 μL) and isobutyl chloroformate (13 μL) were added with the help of a syringe in tandem into a solution of tubulysin B (82 mg) in anhydrous EtOAc (2.0 mL) at −15° C. After stirring for 45 minutes at −15° C. under argon, to the reaction mixture was added a solution of EC0311 in anhydrous EtOAc (1.0 mL). The resulting solution was stirred under argon at −15° C. for 15 minutes and room temperature for an additional 45 minutes, concentrated, and the residue was purified by flash chromatography (silica gel, 2 to 8% MeOH in DCM) to give EC0312 as a white solid (98 mg). The foregoing method is equally applicable for preparing other tubulysin derivatives by the appropriate selection of the tubulysin starting compound.

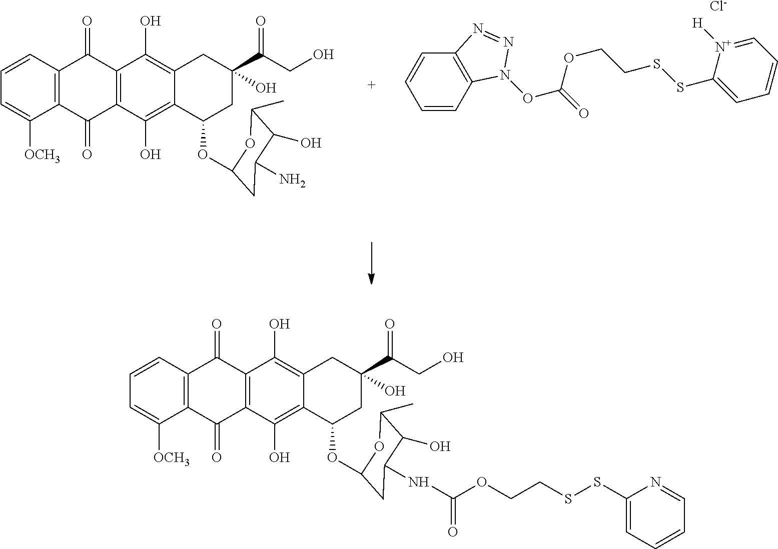 [SMILES: [Cl-] ; COc1cccc2c1C(=O)c1c(O)c3c(c(O)c1C2=O)C[C@@](O)(C(=O)CO)C[C@@H]3OC1CC(N)C(O)C(C)O1 ; O=C(OCCSSc1cccc[nH+]1)On1nnc2ccccc21 ; COc1cccc2c1C(=O)c1c(O)c3c(c(O)c1C2=O)C[C@@](O)(C(=O)CO)C[C@@H]3OC1CC(NC(=O)OCCSSc2ccccn2)C(O)C(C)O1]

Hydroxydaunorubucin pyridyldisulfide. Similarly, this compound was prepared as described herein in 65% yield, and according to the foregoing scheme.

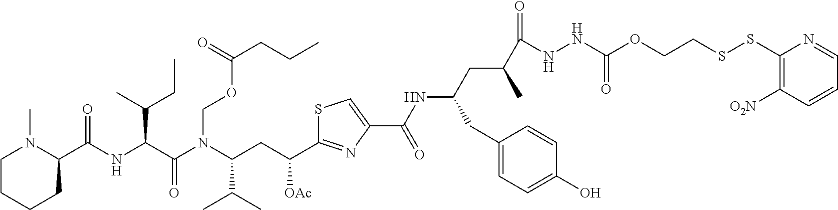 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSc2ncccc2[N+](=O)[O-])cs1)C(C)C]

Tubulysin B nitropyridyldisulfide. Similarly, this compound was prepared as described herein.

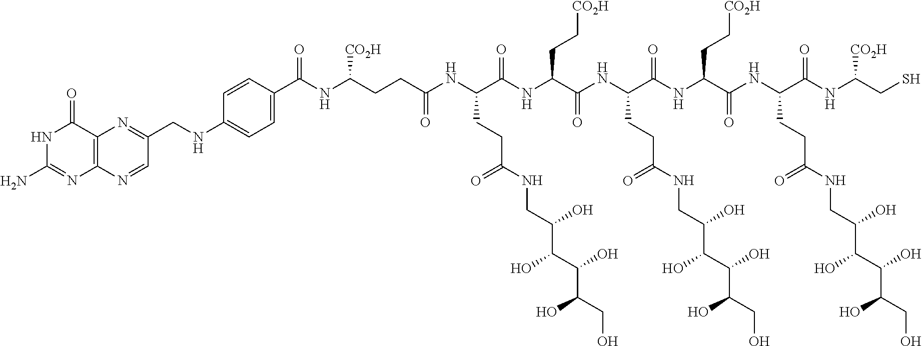 [SMILES: Nc1nc2ncc(CNc3ccc(C(=O)N[C@@H](CCC(=O)N[C@@H](CCC(=O)NC[C@H](O)[C@@H](O)[C@H](O)[C@H](O)CO)C(=O)N[C@@H](CCC(=O)O)C(=O)N[C@@H](CCC(=O)NC[C@H](O)[C@@H](O)[C@H](O)[C@H](O)CO)C(=O)N[C@@H](CCC(=O)O)C(=O)N[C@@H](CCC(=O)NC[C@H](O)[C@@H](O)[C@H](O)[C@H](O)CO)C(=O)N[C@@H](CS)C(=O)O)C(=O)O)cc3)nc2c(=O)[nH]1]

EC0488. This compound was prepared by SPPS according to the general peptide synthesis procedure described herein starting from H-Cys(4-methoxytrityl)-2-chlorotrityl-Resin, and the following SPPS reagents:

| Reagents | mmol | equivalent | MW | amount |
|---|---|---|---|---|
| H-Cys(4-methoxytrityl)-2- | 0.10 | | | 0.17 g |
| chlorotrityl-Resin (loading | | | | |
| 0.6 mmol/g) | | | | |
| EC0475 | 0.13 | 1.3 | 612.67 | 0.082 g |
| Fmoc-Glu(OtBu)-OH | 0.19 | 1.9 | 425.47 | 0.080 g |
| EC0475 | 0.13 | 1.3 | 612.67 | 0.082 g |
| Fmoc-Glu(OtBu)-OH | 0.19 | 1.9 | 425.47 | 0.080 g |
| EC0475 | 0.13 | 1.3 | 612.67 | 0.082 g |
| Fmoc-Glu-OtBu | 0.19 | 1.9 | 425.47 | 0.080 g |
| N¹⁰TFA-Pteroic Acid | 0.16 | 1.6 | 408.29 | 0.066 g |
| (dissolve in 10 ml DMSO) | | | | |
| DIPEA | | 2.0 eq of AA | | |
| PyBOP | | 1.0 eq of AA | | |

Coupling steps. In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3× (10 min), before each amino acid coupling. Continue to complete all 9 coupling steps. At the end treat the resin with 2% hydrazine in DMF 3× (5 min) to cleave TFA protecting group on Pteroic acid, wash the resin with DMF (3×), IPA (3×), MeOH (3×), and bubble the resin with argon for 30 min.

Cleavage step. Reagent: 92.5% TFA, 2.5% H₂O, 2.5% triisopropylsilane, 2.5% ethanedithiol. Treat the resin with cleavage reagent 3× (10 min, 5 min, 5 min) with argon bubbling, drain, wash the resin once with cleavage reagent, and combine the solution. Rotavap until 5 ml remains and precipitate in diethyl ether (35 mL). Centrifuge, wash with diethyl ether, and dry. About half of the crude solid (−100 mg) was purified by HPLC.

HPLC Purification step. Column: Waters Xterra Prep MS C18 10 μm 19×250 mm; Solvent A: 10 mM ammonium acetate, pH 5; Solvent B: ACN; Method: 5 min 0% B to 25 min 20% B 26 mL/min. Fractions containing the product was collected and freeze-dried to give 43 mg EC0488 (51% yield). ¹H NMR and LC/MS (exact mass 1678.62) were consistent with the product.

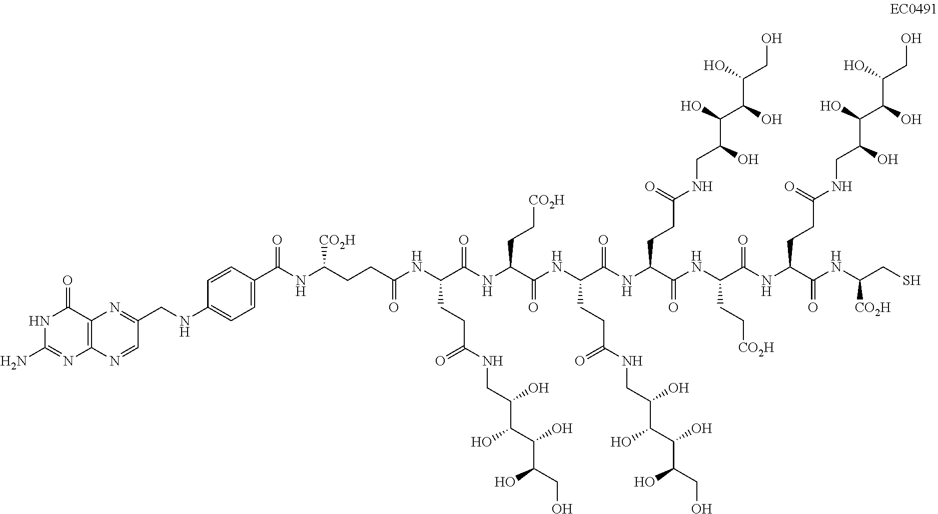 [SMILES: Nc1nc2ncc(CNc3ccc(C(=O)N[C@@H](CCC(=O)N[C@@H](CCC(=O)NC[C@H](O)[C@@H](O)[C@H](O)[C@H](O)CO)C(=O)N[C@@H](CCC(=O)O)C(=O)N[C@@H](CCC(=O)NC[C@H](O)[C@@H](O)[C@H](O)[C@H](O)CO)C(=O)N[C@@H](CCC(=O)NC[C@H](O)[C@@H](O)[C@H](O)[C@H](O)CO)C(=O)N[C@@H](CCC(=O)O)C(=O)N[C@@H](CCC(=O)NC[C@H](O)[C@@H](O)[C@H](O)[C@H](O)CO)C(=O)N[C@@H](CS)C(=O)O)C(=O)O)cc3)nc2c(=O)[nH]1]

Preparation of EC0491. This compound was prepared by SPPS according to the general peptide synthesis procedure described herein starting from H-Cys(4-methoxytrityl)-2-chlorotrityl-Resin, and the following SPPS reagents:

| Reagents | mmol | equivalent | MW | amount |
|---|---|---|---|---|
| H-Cys(4-methoxytrityl)-2- | 0.10 | | | 0.17 g |
| chlorotrityl-Resin (loading | | | | |
| 0.6 mmol/g) | | | | |
| EC0475 | 0.13 | 1.3 | 612.67 | 0.080 g |
| Fmoc-Glu(OtBu)-OH | 0.20 | 2.0 | 425.47 | 0.085 g |
| EC0475 | 0.13 | 1.3 | 612.67 | 0.080 g |
| EC0475 | 0.13 | 1.3 | 612.67 | 0.080 g |
| Fmoc-Glu(OtBu)-OH | 0.20 | 2.0 | 425.47 | 0.085 g |
| EC0475 | 0.13 | 1.3 | 612.67 | 0.080 g |
| Fmoc-Glu-OtBu | 0.20 | 2.0 | 425.47 | 0.085 g |
| N¹⁰TFA-Pteroic Acid | 0.25 | 2.5 | 408.29 | 0.105 g |
| (dissolve in 10 ml DMSO) | | | | |
| DIPEA | | 2.0 eq of AA | | |
| PyBOP | | 1.0 eq of AA | | |

Coupling steps. In a peptide synthesis vessel add the resin, add the amino acid solution, DIPEA, and PyBOP. Bubble argon for 1 hr. and wash 3× with DMF and IPA. Use 20% piperidine in DMF for Fmoc deprotection, 3× (10 min), before each amino acid coupling. Continue to complete all 9 coupling steps. At the end treat the resin with 2% hydrazine in DMF 3× (5 min) to cleave TFA protecting group on Pteroic acid, wash the resin with DMF (3×), IPA (3×), MeOH (3×), and bubble the resin with argon for 30 min.

Cleavage step. Reagent: 92.5% TFA, 2.5% H₂O, 2.5% triisopropylsilane, 2.5% ethanedithiol. The resin was treated with the cleavage reagent 3× (10 min, 5 min, 5 min) with argon bubbling, drained, the resin was washed once with cleavage reagent, and the treatment solutions and was were combined. The combined solutions were concentrated under reduced pressure to a volume of 5 mL and treated with diethyl ether (35 mL) to form a precipitate. The precipitate was collected by centrifuge, washed with diethyl ether, and dried. The crude solid was purified by HPLC. HPLC Purification step: column: Waters Xterra Prep MS C₁₈ 10 μm 19×250 mm; solvent A: 10 mM ammonium acetate, pH 5; solvent B: ACN; gradient method: 5 min 0% B to 25 min 20% B 26 mL/min. Fractions containing the product were collected and freeze-dried to give 100 mg of EC0491 (51% yield). ¹H NMR and LC/MS (exact mass 1678.62) were consistent with the product.

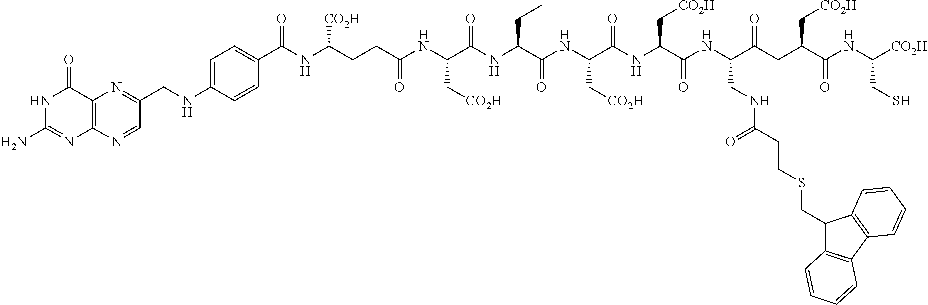 [SMILES: CC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CNC(=O)CCSCC1c2ccccc2-c2ccccc21)C(=O)C[C@@H](CC(=O)O)C(=O)N[C@@H](CS)C(=O)O]

EC0351. Similarly, this compound was prepared as described herein.

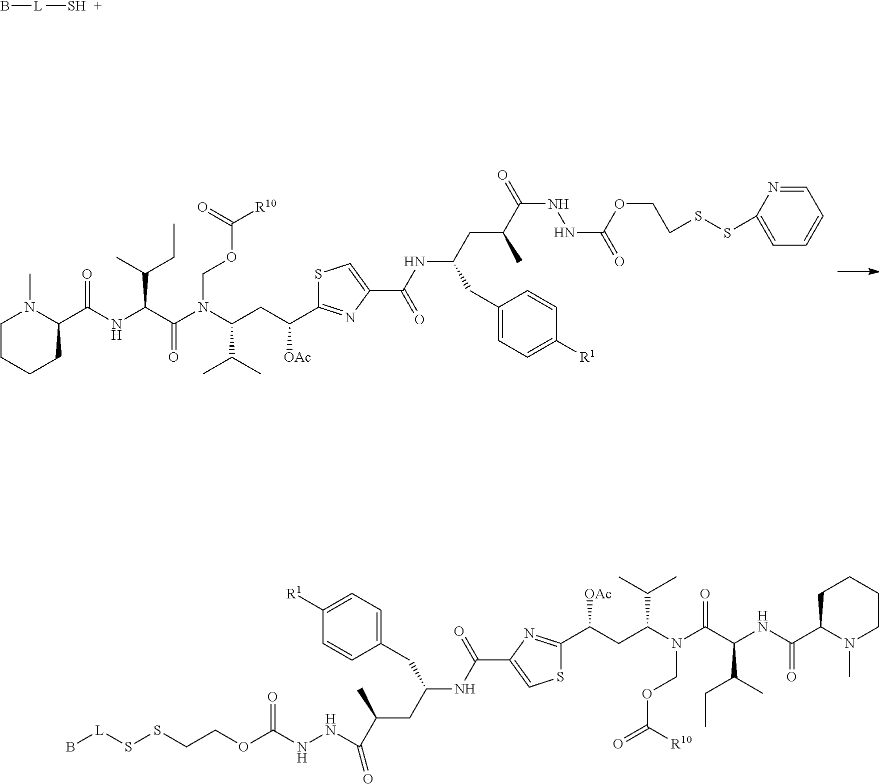

General Synthesis of Disulfide Containing Tubulysin Conjugates. Illustrated with pyridinyl disulfide derivatives of certain naturally occurring tubulysins, where R¹ is hydrogen or OH, and R¹⁰, is alkyl or alkenyl. A binding ligand-linker intermediate containing a thiol group is taken in deionized water (ca. 20 mg/mL, bubbled with argon for 10 minutes prior to use) and the pH of the suspension was adjusted by saturated NaHCO₃ (bubbled with argon for 10 minutes prior to use) to about 6.9 (the suspension may become a solution when the pH increased). Additional deionized water is added (ca. 20-25%) to the solution as needed, and to the aqueous solution is added immediately a solution of EC0312 in THF (ca. 20 mg/mL). The reaction mixture becomes homogenous quickly. After stirring under argon, e.g. for 45 minutes, the reaction mixture is diluted with 2.0 mM sodium phosphate buffer (pH 7.0, ca 150 volume percent) and the THF is removed by evacuation. The resulting suspension is filtered and the filtrate may be purified by preparative HPLC (as described herein). Fraction are lyophilized to isolate the conjugates. The foregoing method is equally applicable for preparing other tubulysin conjugates by the appropriate selection of the tubulysin starting compound.

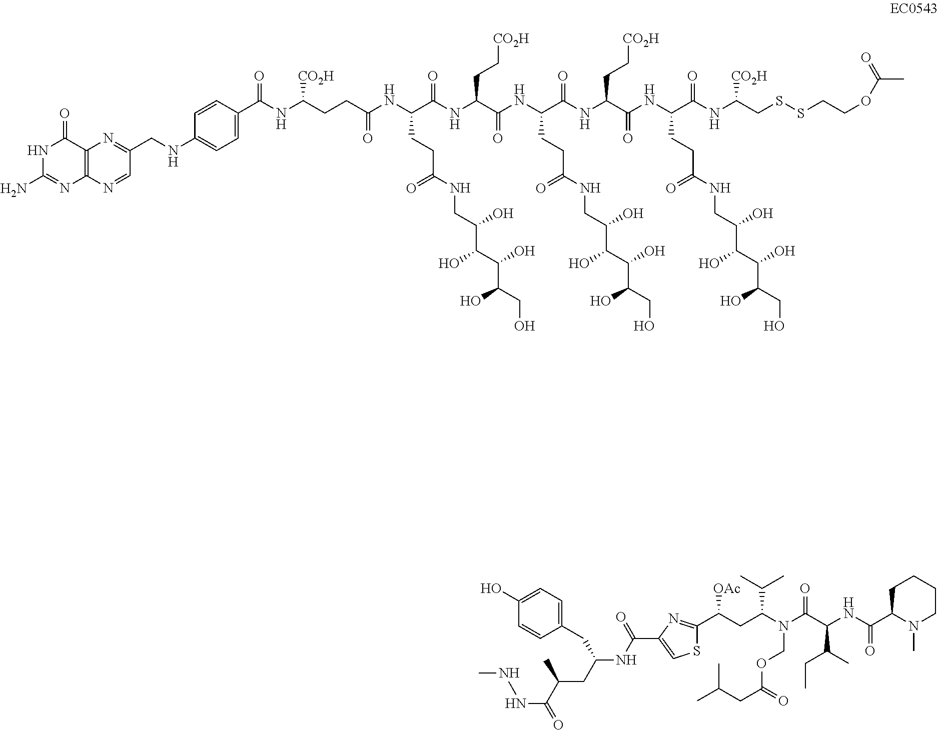

General Method 2 for Preparing Conjugates (one-pot). Illustrated with preparation of EC0543. DIPEA (7.8 μL) and isobutyl chloroformate (3.1 μL) were added with the help of a syringe in tandem into a solution of tubulysin A (18 mg) in anhydrous EtOAc (0.50 mL) at −15° C. After stirring for 35 minutes at −15° C. under argon, to the reaction mixture was added a solution of EC0311 (5.8 mg) in anhydrous EtOAc (0.50 mL). The cooling was removed and the reaction mixture was stirred under argon for an additional 45 minutes, concentrated, vacuumed, and the residue was dissolved in THF (2.0 mL). Meanwhile, EC0488 (40 mg) was dissolved in deionized water (bubbled with argon for 10 minutes prior to use) and the pH of the aqueous solution was adjusted to 6.9 by saturated NaHCO₃. Additional deionized water was added to the EC0488 solution to make a total volume of 2.0 mL and to which was added immediately the THF solution containing the activated tubulysin. The reaction mixture, which became homogeneous quickly, was stirred under argon for 50 minutes and quenched with 2.0 mM sodium phosphate buffer (pH 7.0, 15 mL). The resulting cloudy solution was filtered and the filtrate was injected into a preparative HPLC for purification. Column: Waters XTerra Prep MS C₁₈ 10 μM, 19×250 mm; Mobile phase A: 2.0 mM sodium phosphate buffer, pH 7.0; Mobile phase B: acetonitrile; Method: 1% B for 5 minutes, then 1% B to 60% B over the next 30 minutes, flow rate=26 mL/min. Fractions from 20.75-24.50 minutes were collected and lyophilized to afford EC0543 as a pale yellow fluffy solid (26 mg). The foregoing method is equally applicable for preparing other tubulysin conjugates by the appropriate selection of the tubulysin starting compound.

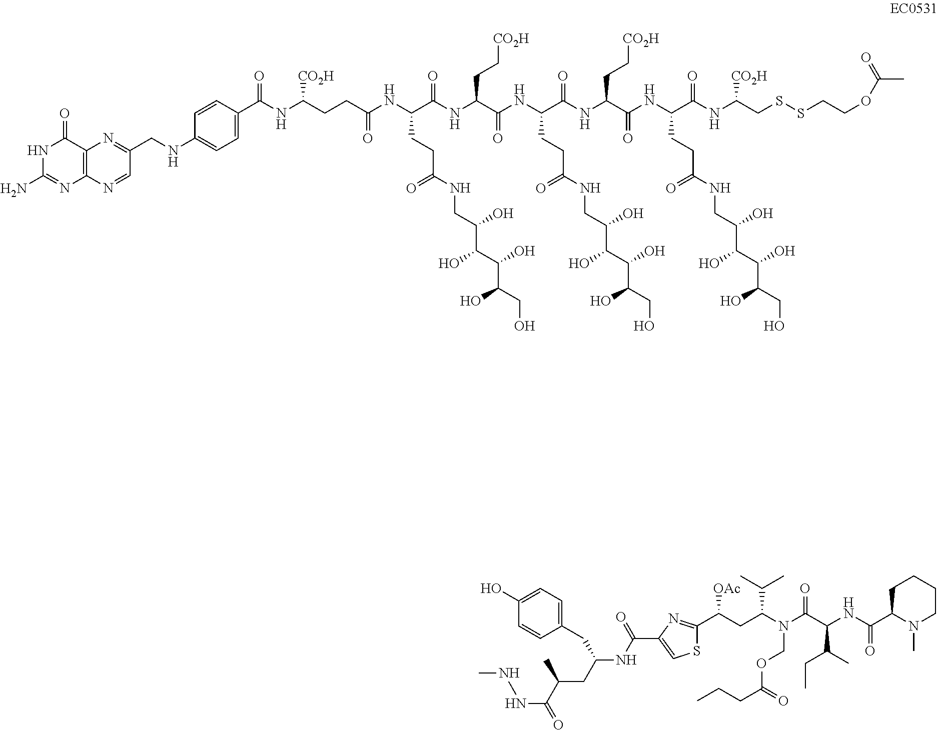

Preparation of EC0531 Example 1. EC0488 (75 mg) was dissolved in 20 mM phosphate buffer (pH 7, 2.2 mL) and to which was added a solution of EC0312 (43 mg) in MeOH (2.2 mL). The resulting homogeneous solution was stirred at RT under argon for 30 min, and then injected directly into a preparatory HPLC for purification. Mobile phase A: 50 mM NH₄HCO₃ buffer, pH 7.0; mobile phase B: acetonitrile; Method: 10% B to 80% B over 20 minutes, flow rate=25 mL/min. Fractions containing the desired product were collected and lyophilized to afford EC0531 as a fluffy yellow solid (41 mg).

Preparation of EC0531 Example 2. A solution of EC0488 (153 mg) in phosphate buffer (4.4 mL, 20 mM, pH 7.0) was added to a solution of EC0312 (78 mg) in MeOH (4.4 mL). The resulting homogenous solution was stirred at RT under argon for 15 min and injected into a preparatory HPLC for purification. Preparative HPLC parameters: column: Waters XTerra Prep MS C18 OBD 5 μm, 19×100 mm; mobile phase A: 20 mM NH₄HCO₃ buffer, pH 7.0; mobile phase B: acetonitrile; method: after loading, a gradient from 10% B to 80% B over 20 minutes at a flow rate of 26 mL/min was run. Fractions containing the desired product were collected and lyophilized to afford 84 mg EC0531 as a pale yellow fluffy solid.

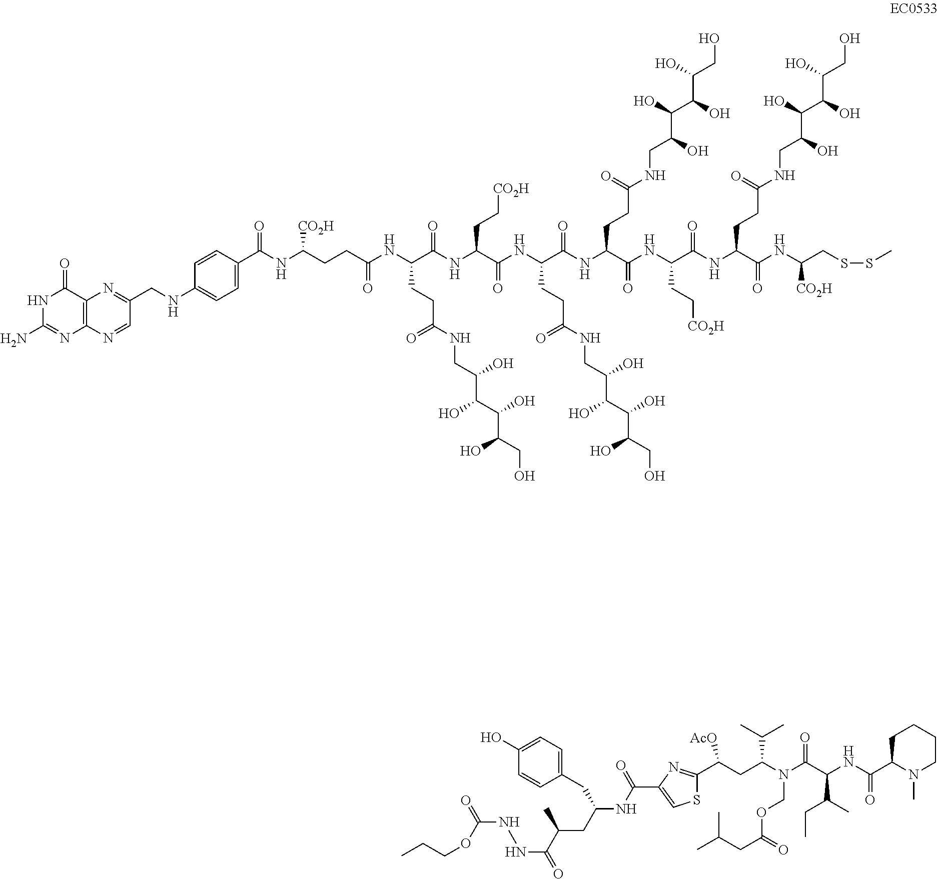

### Preparation of EC0533.

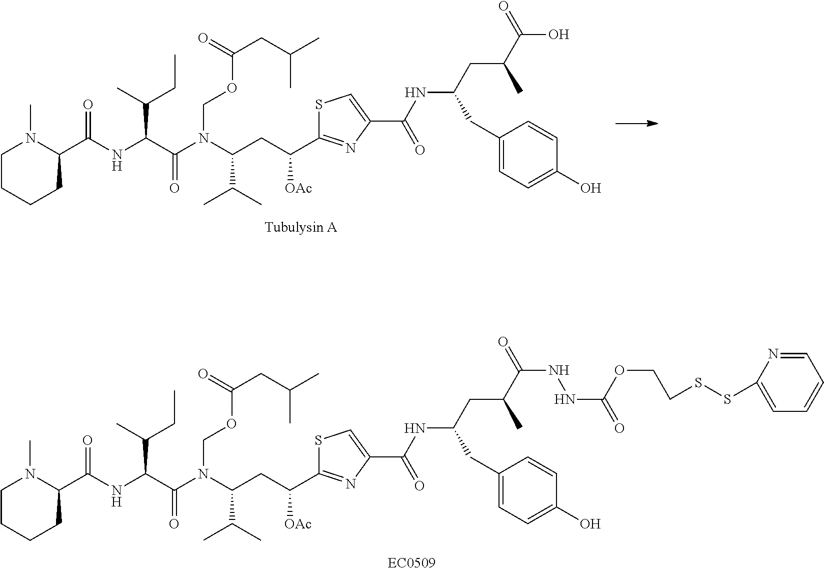 [SMILES: CCC(C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)O)cs1)C(C)C ; CCC(C)[C@H](NC(=O)[C@H]1CCCCN1C)C(=O)N(COC(=O)CC(C)C)[C@H](C[C@@H](OC(C)=O)c1nc(C(=O)N[C@@H](Cc2ccc(O)cc2)C[C@H](C)C(=O)NNC(=O)OCCSSc2ccccn2)cs1)C(C)C]

Step 1. Activation of Tubulysin A. DIPEA (29.5 μL) and isobutyl chloroformate (13.7 μL) were added in tandem via syringe into a solution of tubulysin A (68 mg) in anhydrous EtOAc (2.0 mL) at −15° C. After stirring for 30 minutes under argon (−15° C.˜−10° C.), to the reaction mixture was added to a solution of EC0311 (26.7 mg) in anhydrous EtOAc (1.0 mL). The resulting solution was stirred under argon for an additional 1 hr (−15° C.˜RT), concentrated, and the residue was purified by flash chromatography (silica gel, 1.5 to 5% MeOH in DCM) to give EC0509 as a white solid (66.0 mg).

Step 2) Conjugation. EC0491 (40.0 mg) was added to deionized water (1.8 mL, bubbled with argon for 10 minutes prior to use) and the pH of the suspension was adjusted with saturated NaHCO₃ (bubbled with argon for 10 minutes prior to use) to about pH 6.9 (the suspension became a solution when the pH increased). Additional deionized water was added to the solution to make a total volume of 2.5 mL and to the aqueous solution was added immediately a solution of EC0509 (21.7 mg) in THF (2.5 mL). The reaction mixture became homogenous quickly. After stirring under argon for 1 hr, the reaction mixture was diluted with 2.0 mM sodium phosphate buffer (pH 7.0, 40 mL) and the THF was removed under reduced pressure. The resulting suspension was filtered and the filtrate was injected into a preparative HPLC for purification. HPLC parameters: column: Waters XTerra Prep MS C₁₈ 10 μm, 19×250 mm; mobile phase A: 2.0 mM sodium phosphate buffer, pH 7.0; mobile phase B: acetonitrile; method: 1% B for 5 minutes, then 1% B to 60% B over the next 30 minutes, flow rate=26 mL/min. Fractions containing the desired product were collected and lyophilized to afford 26.4 mg EC0533 as a pale yellow fluffy solid.

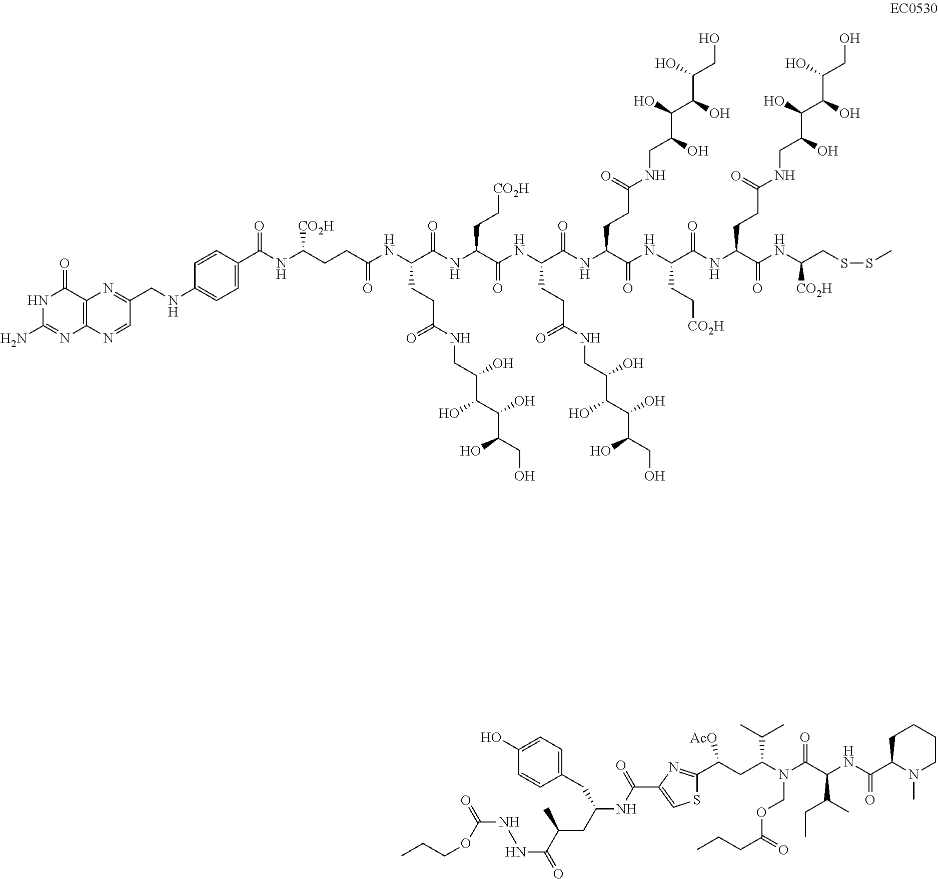

Preparation of EC0530. EC0491 (40 mg) suspended in deionized water (1.8 mL, bubbled with argon for 10 minutes prior to use) and the pH of the suspension was adjusted with saturated NaHCO₃ (bubbled with argon for 10 minutes prior to use) to about 6.9 (the suspension became a solution when the pH increased). Additional deionized water was added to the solution to make a total volume of 2.5 mL and to the aqueous solution was added immediately a solution of EC0312 (21 mg) in THF (2.5 mL). The reaction mixture became homogenous quickly. After stirring under argon for 35 minutes, the reaction mixture was diluted with 2.0 mM sodium phosphate buffer (pH 7.0, 40 mL) and the THF was removed under reduced pressure. The resulting suspension was filtered and the filtrate was injected into a preparative HPLC for purification. HPLC parameters: column: Waters XTerra Prep MS C₁₈ 10 μm, 19×250 mm; mobile phase A: 2.0 mM sodium phosphate buffer, pH 7.0; mobile phase B: acetonitrile; gradient method: 1% B for 5 minutes, then 1% B to 60% B over the next 30 minutes at a flow rate of 26 mL/min. Fractions containing the desired product were collected and lyophilized to afford 37.6 mg EC0530 as a pale yellow fluffy solid.

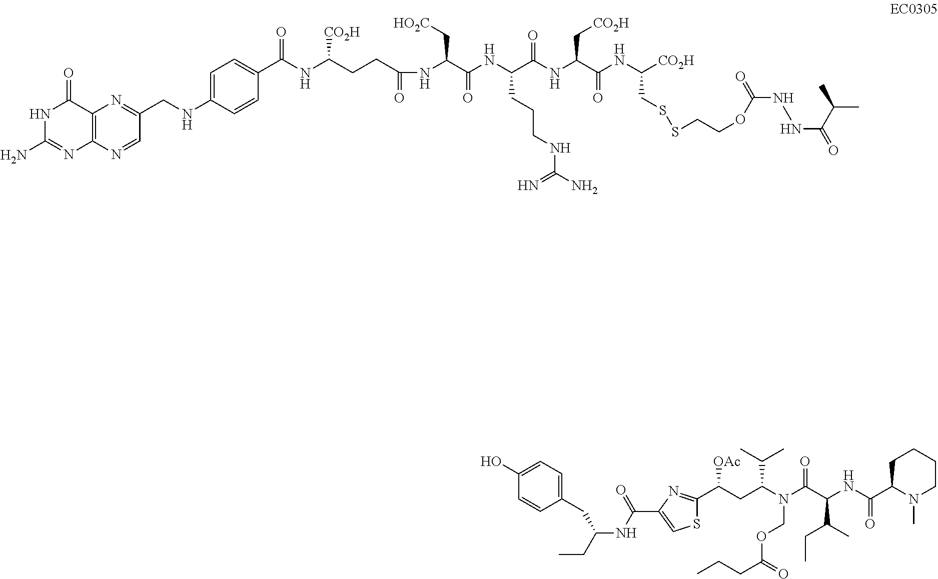

EC0305. EC089 (86 mg) was suspended in deionized water (4.0 mL, bubbled with argon for 10 minutes prior to use) and the pH of the suspension was adjusted by saturated NaHCO₃ (bubbled with argon for 10 minutes prior to use) to about 6.9 (the suspension became a solution when the pH increased). Additional deionized water was added to the solution to make a total volume of 5.0 mL and to the aqueous solution was added immediately a solution of EC0312 (97 mg) in THF (5.0 mL). The reaction mixture became homogenous quickly. After stirring under argon for 45 minutes, the reaction mixture was diluted with 2.0 mM sodium phosphate buffer (pH 7.0, 15 mL) and the THF was removed on a Rotavapor. The resulting suspension was filtered and the filtrate was injected into a preparative HPLC for purification (Column: Waters XTerra Prep MS C₁₈ 10 μm, 19×250 mm; Mobile phase A: 2.0 mM sodium phosphate buffer, pH 7.0; Mobile phase B: acetonitrile; Method: 5% B to 80% B over 25 minutes, flow rate=25 mL/min). Fractions from 10.04-11.90 minutes were collected and lyophilized to give EC0305 as a pale yellow fluffy solid (117 mg).

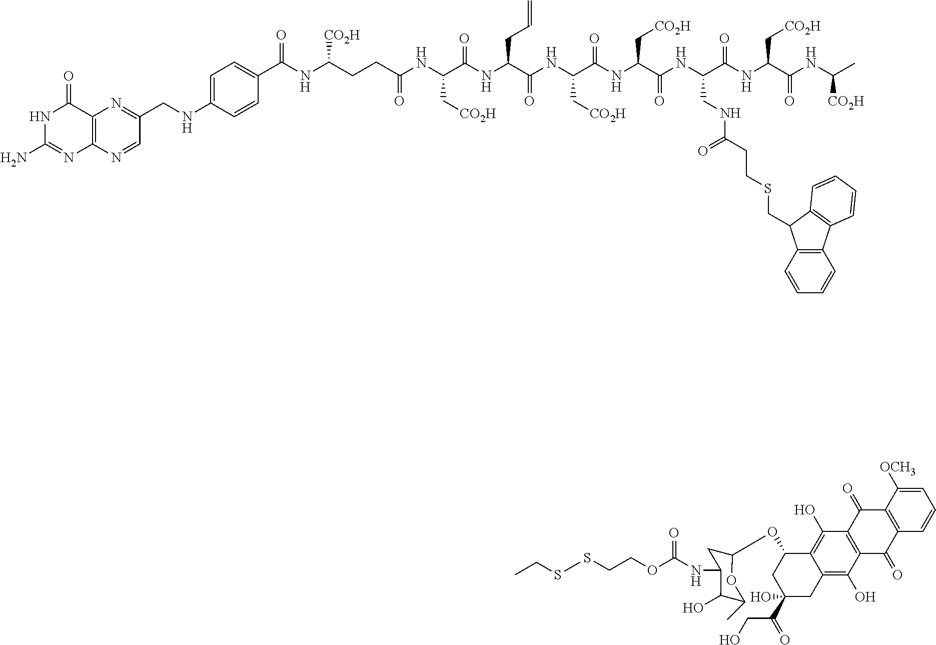 [SMILES: C=CC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CNC(=O)CCSCC1c2ccccc2-c2ccccc21)C(=O)N[C@@H](CC(=O)O)C(=O)N[C@@H](C)C(=O)O ; CCSSCCOC(=O)NC1CC(O[C@H]2C[C@](O)(C(=O)CO)Cc3c(O)c4c(c(O)c32)C(=O)c2c(OC)cccc2C4=O)OC(C)C1O]

EC0352. Similarly, this compound was prepared as described herein. EC0352 was prepared by forming a disulfide bond between hydroxydaunorubucin pyridyldisulfide and EC0351 in 55% yield.

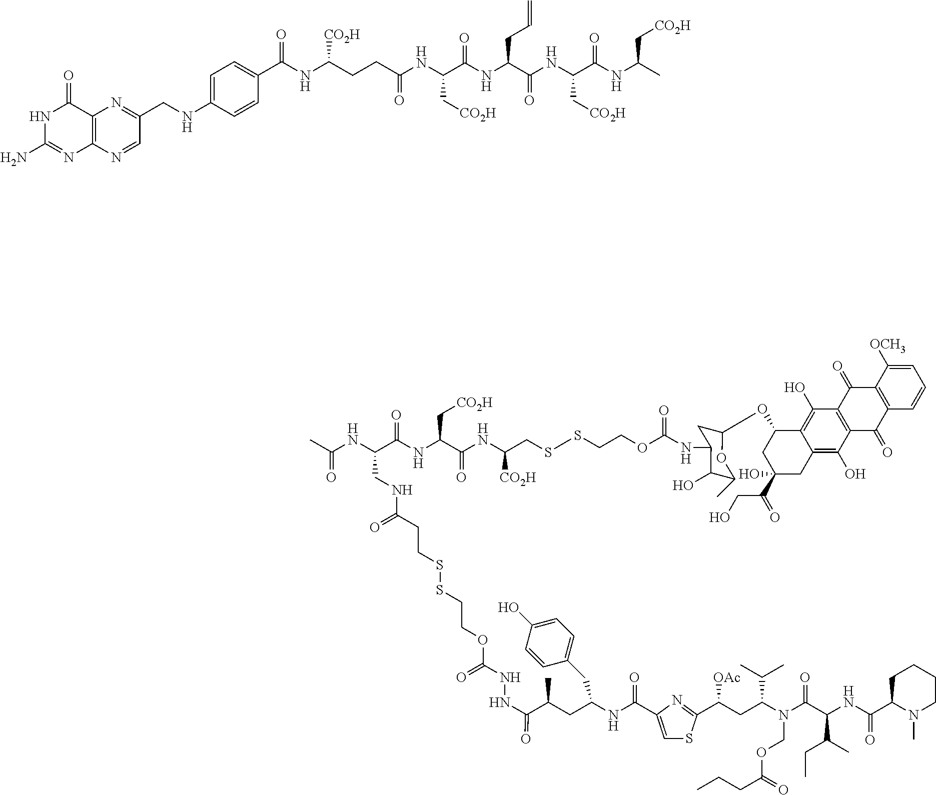 [SMILES: C=CC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@H](C)CC(=O)O ; CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)NNC(=O)OCCSSCCC(=O)NC[C@H](NC(C)=O)C(=O)N[C@@H](CC(=O)O)C(=O)N[C@@H](CSSCCOC(=O)NC2CC(O[C@H]3C[C@](O)(C(=O)CO)Cc4c(O)c5c(c(O)c43)C(=O)c3c(OC)cccc3C5=O)OC(C)C2O)C(=O)O)cs1)C(C)C]

EC0358. Similarly, this compound was prepared as described herein. EC0358 was prepared by forming in DMF/DBU a disulfide bond between EC0352 and tubulysin B pyridyldisulfide in 40% yield.

The following illustrative examples were also prepared using the processes, syntheses, and tubulysins described herein.

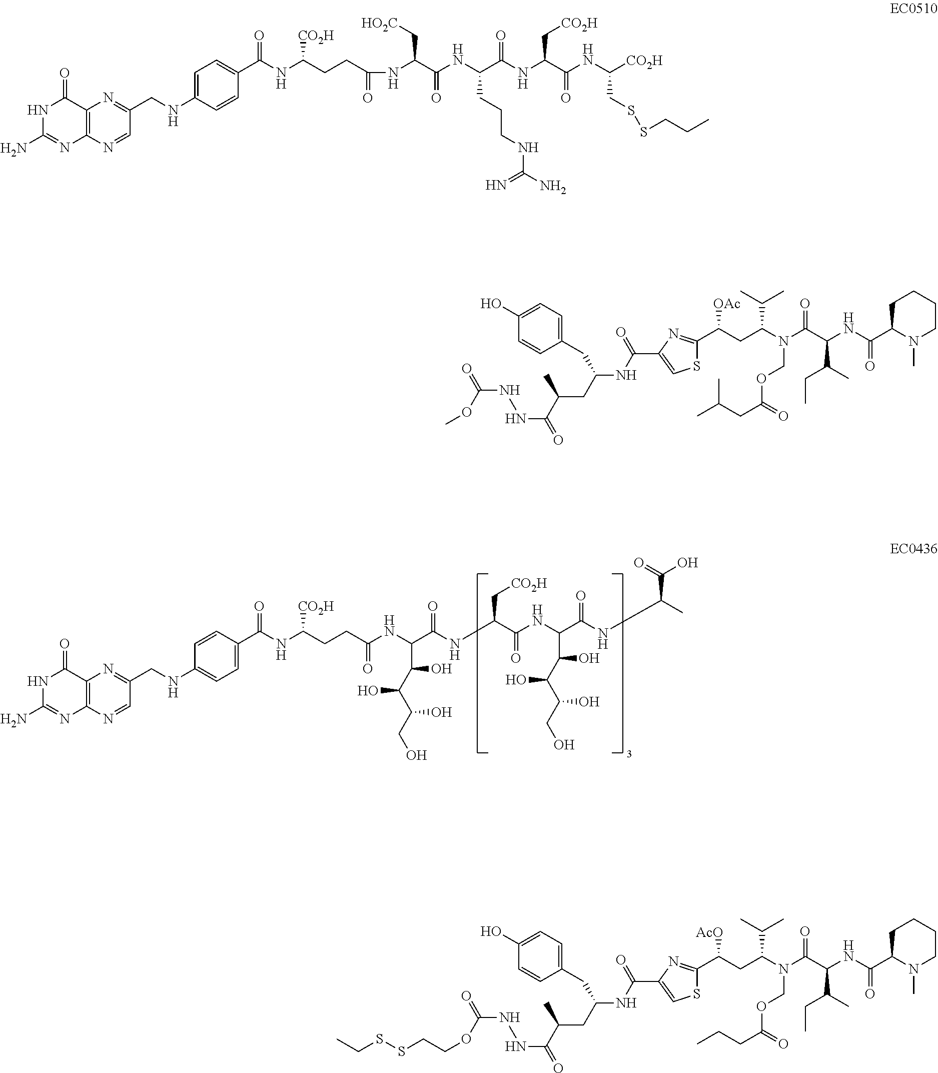

### Method Examples

METHOD: Relative Affinity Assay. The affinity for folate receptors (FRs) relative to folate was determined according to a previously described method (Westerhof, G. R., J. H. Schornagel, et al. (1995) Mol. Pharm. 48: 459-471) with slight modification. Briefly, FR-positive KB cells were heavily seeded into 24-well cell culture plates and allowed to adhere to the plastic for 18 h. Spent incubation media was replaced in designated wells with folate-free RPMI (FFRPMI) supplemented with 100 nM ³H-folic acid in the absence and presence of increasing concentrations of test article or folic acid. Cells were incubated for 60 min at 37° C. and then rinsed 3 times with PBS, pH 7.4. Five hundred microliters of 1% SDS in PBS, pH 7.4, were added per well. Cell lysates were then collected and added to individual vials containing 5 mL of scintillation cocktail, and then counted for radioactivity. Negative control tubes contained only the ³H-folic acid in FFRPMI (no competitor). Positive control tubes contained a final concentration of 1 mM folic acid, and CPMs measured in these samples (representing non-specific binding of label) were subtracted from all samples. Notably, relative affinities were defined as the inverse molar ratio of compound required to displace 50% of ³H-folic acid bound to the FR on KB cells, and the relative affinity of folic acid for the FR was set to 1.

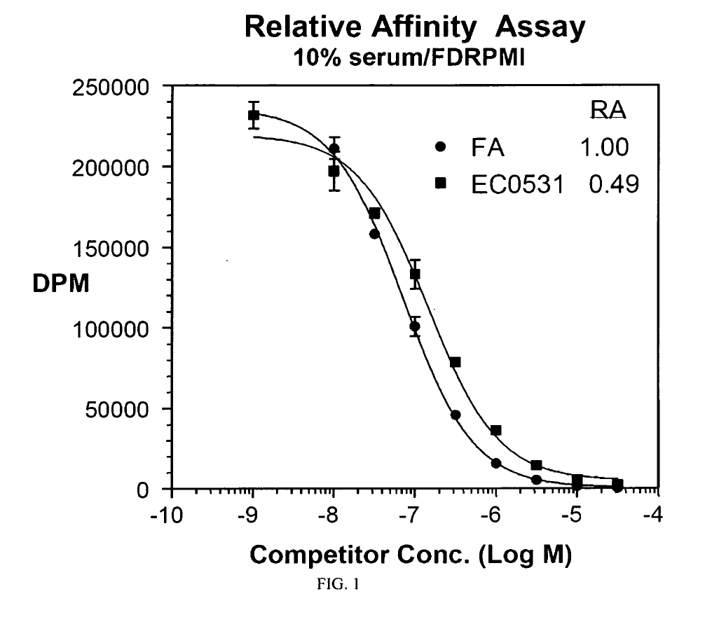

The relative affinity assay results in 10% serum/FDRPMI for EC0531 are shown in the FIG. 1. Compared to folic acid, EC0531 shown 49% relative affinity for the folate receptor.

METHOD: Inhibition of Cellular DNA Synthesis. The compounds described herein were evaluated using an in vitro cytotoxicity assay that predicts the ability of the drug to inhibit the growth of folate receptor-positive KB cells. The compounds were comprised of folate linked to a respective chemotherapeutic drug, as prepared according to the protocols described herein. The KB cells were exposed for up to 7 h at 37° C. to the indicated concentrations of folate-drug conjugate in the absence or presence of at least a 100-fold excess of folic acid. The cells were then rinsed once with fresh culture medium and incubated in fresh culture medium for 72 hours at 37° C. Cell viability was assessed using a ³H-thymidine incorporation assay. For compounds described herein, dose-dependent cytotoxicity was generally measurable, and in most cases, the IC₅₀ values (concentration of drug conjugate required to reduce ³H-thymidine incorporation into newly synthesized DNA by 50%) were in the low nanomolar range. Furthermore, the cytotoxicities of the conjugates were reduced in the presence of excess free folic acid, indicating that the observed cell killing was mediated by binding to the folate receptor.

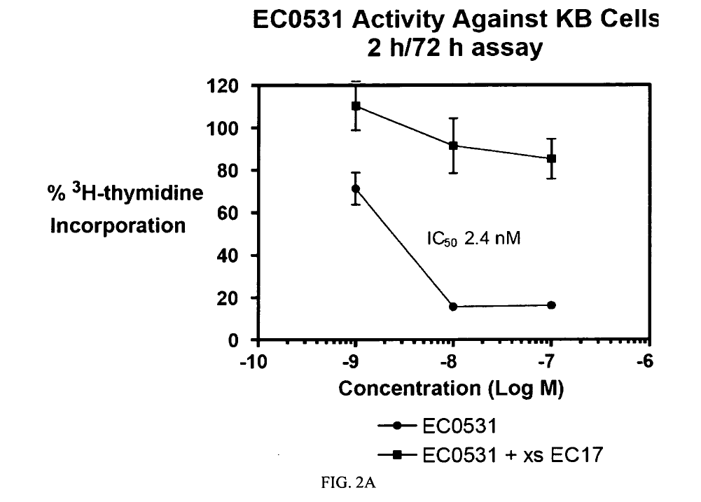

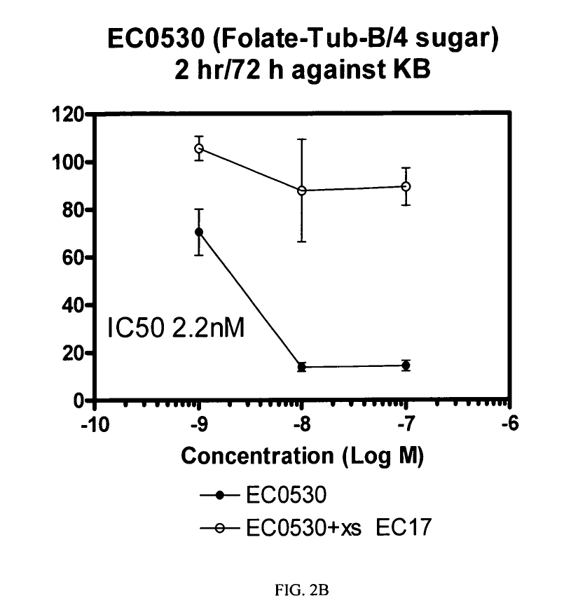

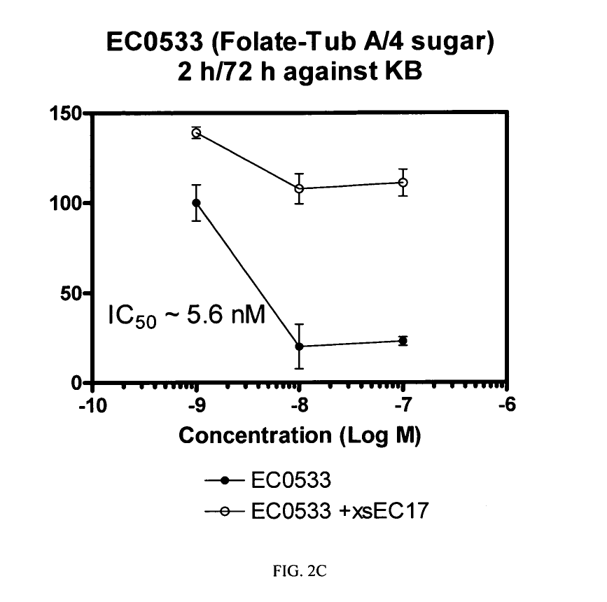

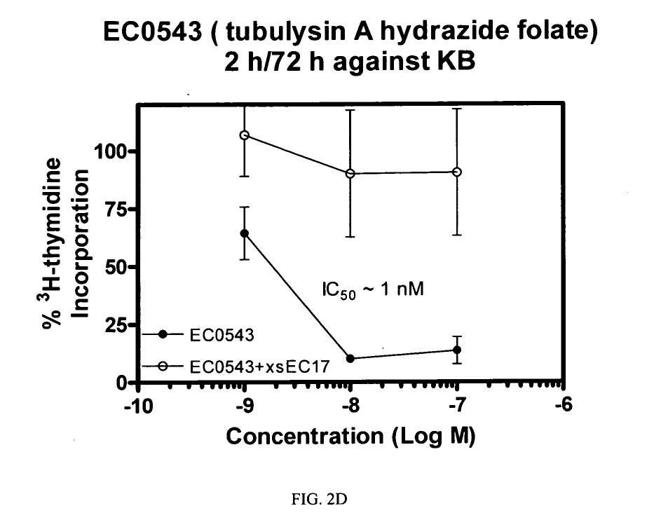

For example, EC0531 exhibited dose-responsive behavior and specificity for the folate receptor after a 2 hour pulse and a 72 hour chase, as shown in the FIG. 2A. The IC₅₀ for EC0531 was about 2.4 nM. In addition, the cytotoxic activity of EC0531 was blocked in the presence of an excess of folic acid, as also shown in FIG. 2A. These results suggest that EC0531 is acting through a folate selective or folate specific mechanism.

METHOD: In vitro test against the various cancer cell lines. Cells are heavily seeded in 24-well Falcon plates and allowed to form nearly confluent monolayers overnight. Thirty minutes prior to the addition of the test compound, spent medium is aspirated from all wells and replaced with fresh folate-deficient RPMI medium (FFRPMI). A subset of wells are designated to receive media containing 100 μM folic acid. The cells in the designated wells are used to determine the targeting specificity. Without being bound by theory it is suggested that the cytotoxic activity produced by test compounds in the presence of excess folic acid, i.e. where there is competition for FR binding, corresponds to the portion of the total activity that is unrelated to FR-specific delivery. Following one rinse with 1 mL of fresh FFRPMI containing 10% heat-inactivated fetal calf serum, each well receives 1 mL of medium containing increasing concentrations of test compound (4 wells per sample) in the presence or absence of 100 μM free folic acid as indicated. Treated cells are pulsed for 2 h at 37° C., rinsed 4 times with 0.5 mL of media, and then chased in 1 mL of fresh medium up to 70 h. Spent medium is aspirated from all wells and replaced with fresh medium containing 5 μCi/mL ³H-thymidine. Following a further 2 h 37° C. incubation, cells are washed 3 times with 0.5 mL of PBS and then treated with 0.5 mL of ice-cold 5% trichloroacetic acid per well. After 15 min, the trichloroacetic acid is aspirated and the cell material solubilized by the addition of 0.5 mL of 0.25 N sodium hydroxide for 15 min. A 450 μL aliquot of each solubilized sample is transferred to a scintillation vial containing 3 mL of Ecolume scintillation cocktail and then counted in a liquid scintillation counter. Final tabulated results are expressed as the percentage of ³H-thymidine incorporation relative to untreated controls.

Each of the cell lines is commercially available except for 4T-1 parent and 4T-1-FR, which were obtained from Rhone Poulenc Rorer.

METHOD: Serum binding against different species. Compounds are tested with 30K NMWL, subjected to Microcon filtration (10,000 g for 30 minutes), and compounds are detected by HPLC. EC0305 was tested against various animal sera and exhibited low serum binding in various species, as shown in the TABLE 1. In particular, EC0305 showed 79.3% binding in human serum and 63.8% binding in mouse serum. EC531 showed 62% binding in human serum and 49.7% binding in mouse serum. There was two times the amount of free EC0531 in serum compared to EC0305.

TABLE 1
Serum Binding of Tubulysin Conjugates

| | Human Serum | | Mouse Serum | |
|---|---|---|---|---|
| Conjugate | % Bound | SD | % Bound | SD |
| EC0305 | 79.3% | 1.7 | 63.8% | 2.3 |
| EC0531 | 62.0% | 2.0 | 49.7% | 2.2 |

50 μM test article concentration in serum; 30K NMWL filtration/HPLC-UV detection
n = 3

METHOD: Inhibition of Tumor Growth in Mice. Four to seven week-old mice (Balb/c or nu/nu strains) were purchased from Harlan Sprague Dawley, Inc. (Indianapolis, Ind.). Normal rodent chow contains a high concentration of folic acid (6 mg/kg chow); accordingly, mice used were maintained on the folate-free diet (Harlan diet #TD00434) for 1 week before tumor implantation to achieve serum folate concentrations close to the range of normal human serum. For tumor cell inoculation, 1×10⁶ M109 cells (Balb/c strain) or 1×10⁶ KB cells (nu/nu strain) in 100 μL were injected in the subcutis of the dorsal medial area. Tumors were measured in two perpendicular directions every 2-3 days using a caliper, and their volumes were calculated as 0.5×L×W², where L=measurement of longest axis in mm and W=measurement of axis perpendicular to L in mm. Log cell kill (LCK) and treated over control (T/C) values were then calculated according to published procedures (see, e.g., Lee et al., “BMS-247550: a novel epothilone analog with a mode of action similar to paclitaxel but possessing superior antitumor efficacy” Clin Cancer Res 7:1429-1437 (2001); Rose, “Taxol-based combination chemotherapy and other in vivo preclinical antitumor studies” J Natl Cancer Inst Monogr 47-53 (1993)). Dosing solutions were prepared fresh each day in PBS and administered through the lateral tail vein of the mice. Dosing was initiated when the s.c. tumors had an average volume between 50-100 mm³ (t₀), typically 8 days post tumor inoculation (PTI) for KB tumors, and 11 days PTI for M109 tumors.

METHOD: General KB Tumor Assay. The anti-tumor activity of the compounds described herein, when administered intravenously (i.v.) to tumor-bearing animals, was evaluated in nu/nu mice bearing subcutaneous KB tumors. Approximately 8 days post tumor inoculation in the subcutis of the right axilla with 1×10⁶ KB cells (average tumor volume at tₒ=50-100 mm³), in mice (5/group) were injected i.v. three times a week (TIW), for 3 weeks with 5 μmol/kg of the drug delivery conjugate or with an equivalent dose volume of PBS (control), unless otherwise indicated. Tumor growth was measured using calipers at 2-day or 3-day intervals in each treatment group. Tumor volumes were calculated using the equation V=a×b²/2, where “a” is the length of the tumor and “b” is the width expressed in millimeters.

METHOD: Additional In vivo antitumor method. Four to six week-old female nu/nu mice (Charles River, Wilmington, Mass.) were maintained on a standard 12 h light-dark cycle and fed ad libitum with folate-deficient chow (Harlan diet #TD00434, Harlan Teklad, Madison, Wis.) for the duration of the experiment. KB cells (1×10⁶ per nu/nu mouse) in 100 μL were injected in the subcutis of the dorsal medial area. Mice were divided into groups of five, and test articles were freshly prepared and injected through the lateral tail vein under sterile conditions in a volume of 200 μL of phosphate-buffered saline (PBS). Intravenous (i.v.) treatments were typically initiated on approximately 9 post-tumor cell implantation when the KB tumors were approximately 100-200 mm³ in volume. The mice in the control groups received no treatment. Growth of each s.c. tumor was followed by measuring the tumor three times per week during treatment and twice per week thereafter until a volume of 1500 mm³ was reached. Tumors were measured in two perpendicular directions using Vernier calipers, and their volumes were calculated as 0.5×L×W², where L=measurement of longest axis in mm and W=measurement of axis perpendicular to L in mm. As a general measure of toxicity, changes in body weights were determined on the same schedule as tumor volume measurements. Survival of animals was monitored daily. Animals that were moribund (or unable to reach food or water) were euthanized by CO₂ asphyxiation. All in vivo studies were performed in accordance with the American Accreditation Association of Laboratory Animal Care guidelines.

For individual tumors, a partial response (PR) was defined as volume regression >50% but with measurable tumor (>2 mm³) remaining at all times. Complete response (CR) was defined as a disappearance of measurable tumor mass (<2 mm³) at some point until the end of the study. Cures were defined as CR's without tumor re-growth within the study time frame. For each treatment group these results are reports {number of animals with a tumor showing partial response, number of animals with a tumor showing complete response, number of animals showing cures}.

METHOD: General M109 Tumors Assay. The anti-tumor activity of the compounds described herein, when administered intravenously (i.v.) to tumor-bearing animals, was evaluated in Balb/c mice bearing subcutaneous M109 tumors (a syngeneic lung carcinoma). Approximately 11 days post tumor inoculation in the subcutis of the right axilla with 1×10⁶ M109 cells (average tumor volume at tₒ=60 mm³), mice (5/group) were injected i.v. three times a week (TIW), for 3 weeks with 1500 nmol/kg of the drug delivery conjugate or with an equivalent dose volume of PBS (control). Tumor growth was measured using calipers at 2-day or 3-day intervals in each treatment group. Tumor volumes were calculated using the equation V=a×b²/2, where “a” is the length of the tumor and “b” is the width expressed in millimeters.

METHOD: General 4T-1 Tumor Assay. Six to seven week-old mice (female Balb/c strain) were obtained from Harlan, Inc., Indianapolis, Ind. The mice were maintained on Harlan's folate-free chow for a total of three weeks prior to the onset of and during this experiment. Folate receptor-negative 4T-1 tumor cells (1×10⁶ cells per animal) were inoculated in the subcutis of the right axilla. Approximately 5 days post tumor inoculation when the 4T-1 tumor average volume was ˜100 mm³, mice (5/group) were injected i.v. three times a week (TIW), for 3 weeks with 3 mol/kg of drug delivery conjugate or with an equivalent dose volume of PBS (control), unless otherwise indicated herein. Tumor growth was measured using calipers at 2-day or 3-day intervals in each treatment group. Tumor volumes were calculated using the equation V=a×b²/2, where “a” is the length of the tumor and “b” is the width expressed in millimeters.

METHOD: Toxicity as Measured by Weight Loss. The percentage weight change of the mice was determined in mice (5 mice/group) on selected days post-tumor inoculation (PTI), and graphed.

METHOD: Alternate dosing schedule. Each of the foregoing assays may be modified as follows: approximately 8 days post tumor inoculation in the subcutis of the right axilla with 1×10⁶ KB cells (average tumor volume at tₒ=50-100 mm³), mice (5/group) are injected i.v. three times a week (TIW), for 3 weeks with a drug delivery conjugate described herein, or with an equivalent dose volume of PBS as control. Tumor growth is measured using calipers at 2-day or 3-day intervals in each treatment group. Tumor volumes were calculated using the equation V=a×b²/2, where “a” is the length of the tumor and “b” is the width expressed in millimeters.

METHOD: Alternate dosing schedule. Each of the foregoing assays may be modified as follows: approximately 8 days post tumor inoculation in the subcutis of the right axilla with 1×10⁶ KB cells (average tumor volume at tₒ=50-100 mm³), mice (5/group) are injected i.v. five times a week on Monday through Friday for 2 or 3 weeks with a drug delivery conjugate described herein, or with an equivalent dose volume of PBS as control. Tumor growth is measured using calipers at 2-day or 3-day intervals in each treatment group. Tumor volumes were calculated using the equation V=a×b²/2, where “a” is the length of the tumor and “b” is the width expressed in millimeters.

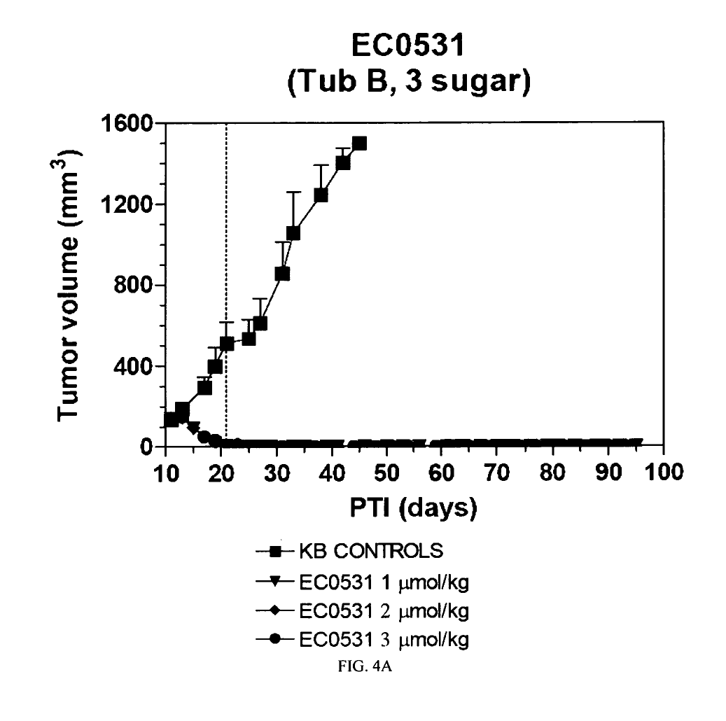

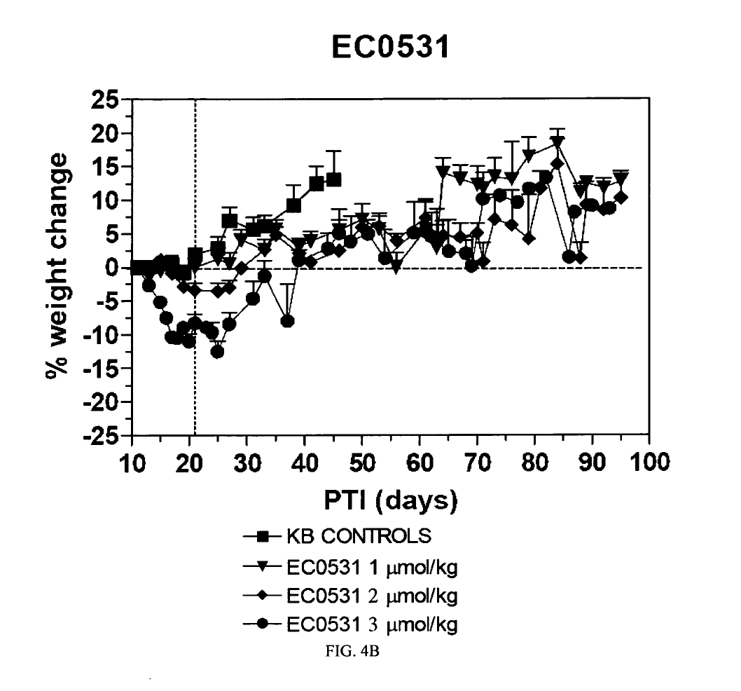

EC0531 was tested at TIW on a two week schedule at various doses, and showed complete responses in 5 of 5 animals tested at a dose at or above 1 μmol/kg, as shown in the FIG. 4. In FIG. 4, Panels A and B, the vertical dotted line indicates the last day of dosing. In addition, no recurrence or regrowth of the tumors was observed during the entire observation period for those doses in 5 of 5 animals, despite that the last administration of conjugate was given more than 70 days earlier, as also shown in the FIG. 4A.

In addition, the observed activity occurred in the apparent absence of weight loss or major organ tissue degeneration, as shown in the FIG. 4B, where the vertical dotted line indicates the last day of dosing.

In contrast to the results observed for the conjugates described herein, the unconjugated tubulysin B free drug

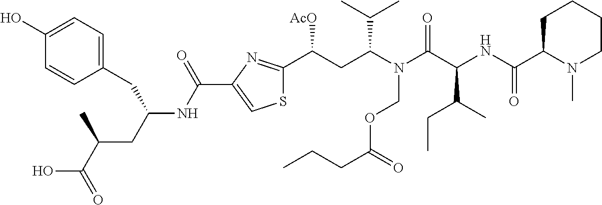 [SMILES: CCCC(=O)OCN(C(=O)[C@@H](NC(=O)[C@H]1CCCCN1C)C(C)CC)[C@H](C[C@@H](OC(C)=O)c1nc(C(=O)N[C@@H](Cc2ccc(O)cc2)C[C@H](C)C(=O)O)cs1)C(C)C]

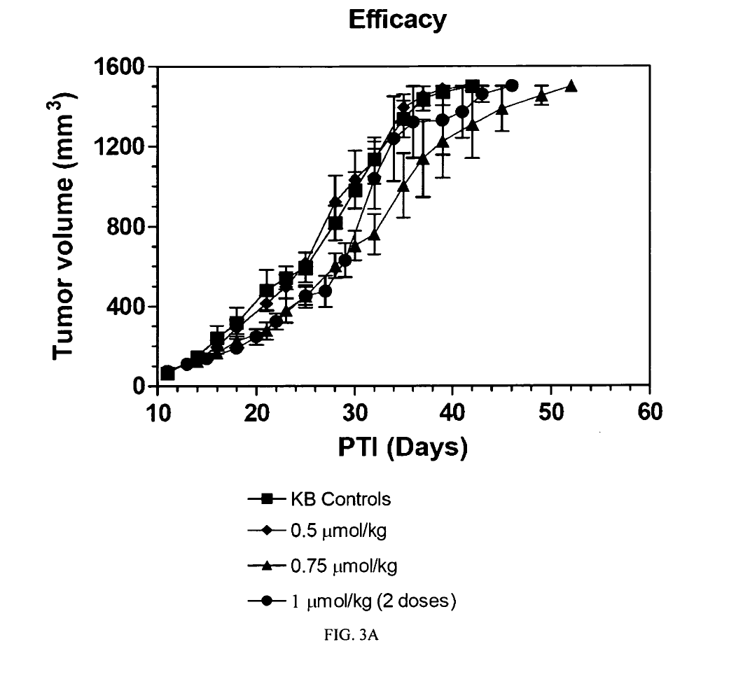

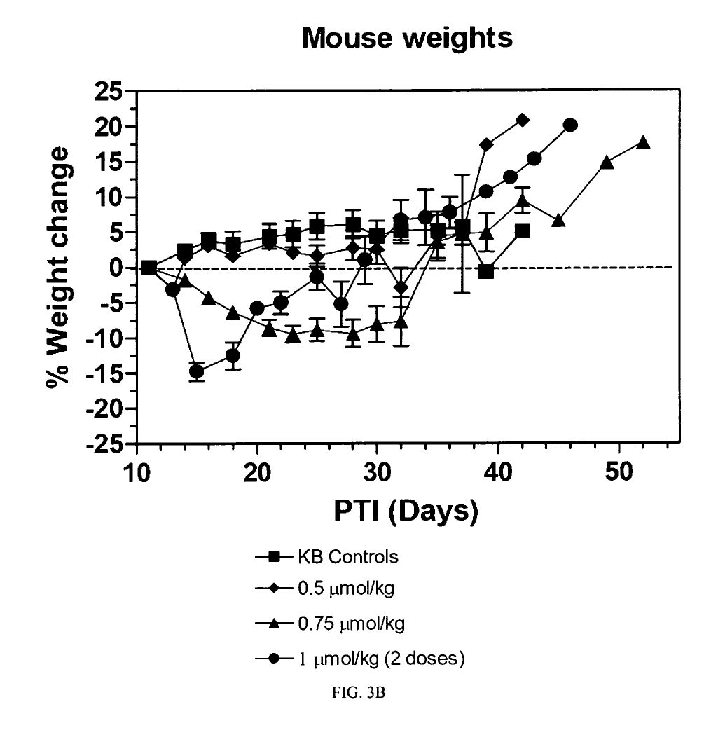

was found to be inactive (0/5 responses) at both tolerable and highly toxic dose levels, as shown in the FIG. 3A (dosing was terminated early in each cohort due to excessive toxicity of the unconjugated drug). FIG. 3B shows the dramatic change in percent body weight of animals treated with unconjugated tubulysin B, as compared to controls. As indicated in FIGS. 3A and 3B, dosing was terminated early in each cohort due to excessive toxicity of the unconjugated drug.

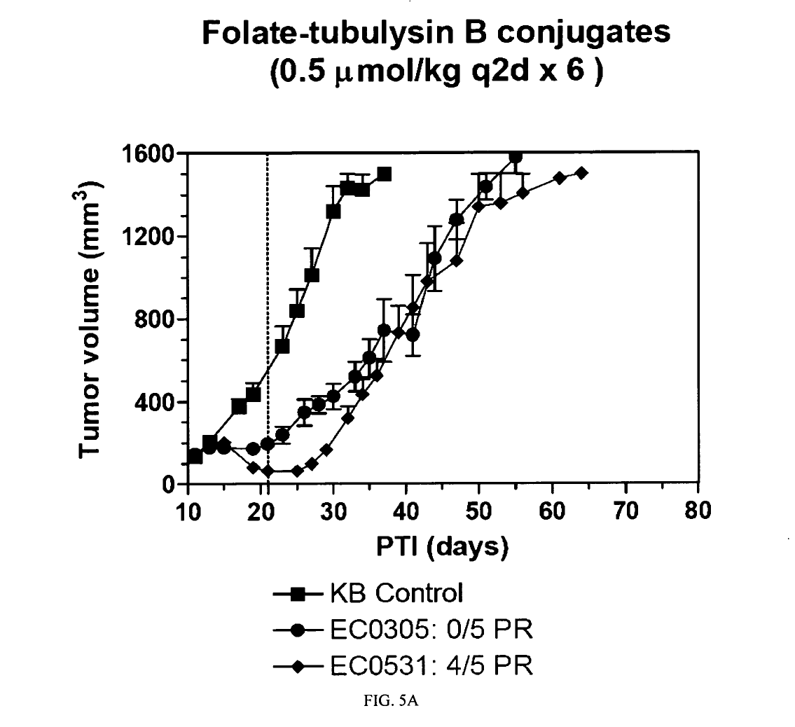

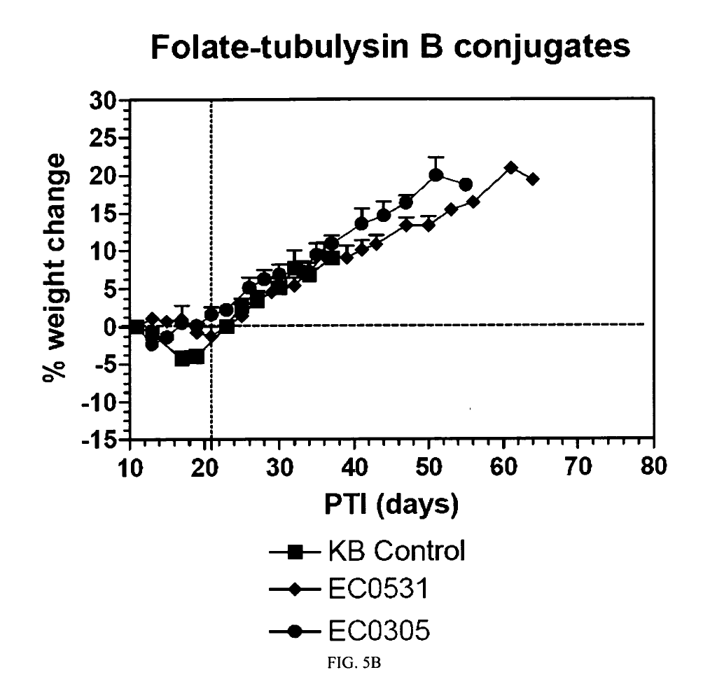

FIG. 5 shows the relative activity of two different tubulysin conjugates, EC0305 and EC0531, on KB tumors compared to controls. Treatment was initiated approximately 8 days after tumor implantation, and each test animal received 0.5 μmol/kg of EC0305 or EC0531 three times per week for two weeks. The vertical dotted line in FIG. 5A shows that the last day of dosing was on day 20. As shown in FIG. 5A, both EC0305 and EC0531 showed complete responses in all animals. However, near about day 35 PTI, the EC0305 treated animals began to show tumor regrowth. In contrast, the EC0531 treated animals not only showed complete responses in 5 of 5 treated animals, but there was no tumor recurrence or regrowth observed in the entire 60-plus day observation period. FIG. 5B shows the percent weight change in treated animals, as compared to controls. In all treated animals, the observed efficacy was not accompanied by any observed gross toxicity as determined by changes in weight of the test animals.

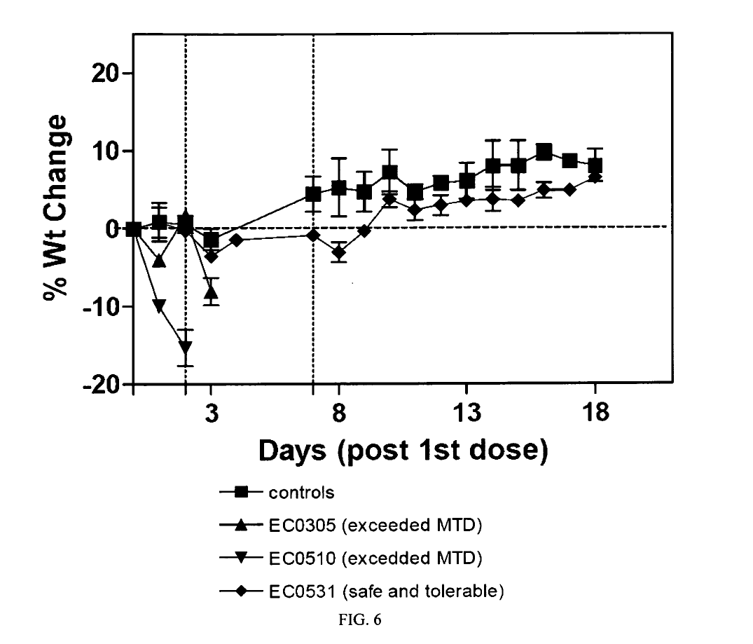

FIG. 6 shows the relative activity of three different tubulysin conjugates, EC0531, EC0305, and EC0510, on KB tumors compared to controls. Treatment was initiated approximately 8 days after tumor implantation, and each test animal received 3 μmol/kg (TIW) of EC0531, EC0305 or EC0510 three times per week for two weeks. The vertical dotted line in FIG. 6 shows that the last days of dosing. FIG. 6 shows the percent weight change in treated animals, as compared to controls. EC0531 was well tolerated at 3 μmol/kg, while EC0310 and EC0305 were not well tolerated by test animals.

The foregoing exemplary embodiments are set forth to provide a more detailed description of certain aspects of the invention described herein. However, the foregoing are intended to be illustrative and accordingly should not be construed as limiting the invention in any way.

1. A method for treating a patient with cancer, the method comprising the step of administering to the patient a composition comprising a conjugate of a tubulysin of the formula
B-L-D
or a pharmaceutically acceptable salt thereof;
wherein
B is a folate;
L is a bivalent linker of the formula 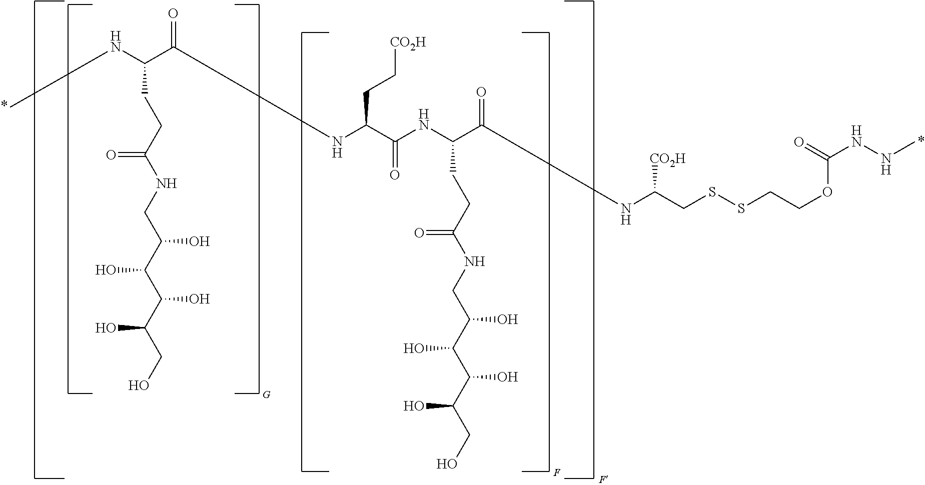
wherein * indicates the points of attachment, and F, F′, and G are each independently 1, 2, 3 or 4; and D is a tubulysin.

2-22. (canceled)
